(19) United States
(12) Patent Application Publication
(10) Pub. No.: US 2026/0250633 A1
(43) Pub. Date: Aug. 27, 2026

(54) METHOD FOR EXPANDING IMMUNE CELLS

(71) Applicant: King's College London, London (GB)

(72) Inventors: Joana F. Neves, London (GB); Geraldine M. Jowett, London (GB); Isabelle Coales, London (GB)

(21) Appl. No.: 18/875,791

(22) PCT Filed: Jun. 16, 2023
(86) PCT No.: PCT/EP2023/066317

§ 371 Date: Dec. 17, 2024

(30) Foreign Application Priority Data
Jun. 17, 2022 (GB) 2208963.5

(51) Int. Cl. C12N 5/078; A61K 35/14; A61K 35/17; C12N 5/0783

(52) U.S. Cl. CPC C12N 5/0634; A61K 35/14; A61K 35/17; C12N 5/0646; C12N 2502/23; C12N 2506/11; C12N 2506/45

(57) ABSTRACT

This invention relates to a method for expanding immune cells, the method comprising co-culturing the immune cells and at least one epithelial organoid, wherein the epithelial organoid comprises more epithelial cells than mesenchymal cells. Also provided are immune cells obtainable by the method, an in vitro population of innate lymphoid cells (ILCs) comprising at least 5×103 ILCs, pharmaceutical compositions and uses thereof.

### TECHNICAL FIELD

This invention relates to a method for expanding immune cells, the method comprising co-culturing the immune cells and at least one epithelial organoid, wherein the epithelial organoid comprises more epithelial cells than mesenchymal cells. Also provided are immune cells obtainable by the method, an in vitro population of innate lymphoid cells (ILCs) comprising at least 5×10³ ILCs, pharmaceutical compositions and uses thereof.

### BACKGROUND

Innate lymphoid cells (ILCs) are mucosal-enriched and tissue-resident cells that mediate mucosal barrier integrity during homeostasis and provide a rapid, antigen non-specific source of cytokines during infection. ILCs currently fall into three groups, comprising five subtypes. The first group encompasses T-bet⁺ Eomes⁺ cytotoxic Natural Killer (NK) cells and T-bet⁺ Eomes⁻ ILC1. The second group comprises Rora⁺ Gata3⁺ ILC2, and the third group encompasses RORγt+Lymphoid tissue inducer cells (LTi) as well as Natural Cytotoxicity Receptor (NCR)^{+/-} ILC3. These categories appear to be less rigidly defined in humans than in mice, and plasticity can occur between groups.

The relative frequency and phenotypes of these ILC subsets varies between tissues in both mice and humans. Understanding what drives this heterogeneity is a topic of considerable interest, as relative ILC frequencies are not only altered during acute infection, but can become chronically dysregulated in inflammatory diseases and cancer, making them attractive targets for therapeutic manipulation. This is especially true as ILC engage in bi-directional interactions with epithelial cells: ILC1 drive epithelial cell proliferation through TGF-β1; ILC2 are activated and proliferate in response to Tuft-cell derived IL-25; and ILC3 drive Lgr5⁺ intestinal stem cell proliferation through IL-22; finally, foetal LTi mediate development of secondary lymphoid structures, whereas NK cells are circulatory and not specifically enriched in mucosa.

Mature ILC stem from the Common Lymphoid Precursor (CLP), which differentiate toward an ILC restricted Lineage-CD127⁺ Id2⁺ IL-7R⁺ α4β7⁺ ILC precursor (ILCP) in mice. Sources of ILCP have been identified in the murine adult bone marrow, foetal liver, small intestine and lung (Bando et al., 2015). ILC precursors have also been identified in human bone marrow, tonsils foetal, paediatric and adult intestines, but these are less well characterised than their murine counterparts (Elmentaite et al., 2021).

Common to mice and humans is the low frequency of ILCs in vivo. Such limited numbers for isolation can hinder attempts to further study ILCs in vitro or in vivo. The therapeutic potential of ILCs is also yet to be realised, given the difficulties in obtaining large enough numbers for cell therapy.

In vitro attempts to produce ILCs have found that CD34⁺ hematopoietic cells can produce ILC when cultured with feeder cells modified to express Notch ligands and with/without IL-15 supplementation (Hernández et al., 2021). However, these approaches do not efficiently drive maturation of all subsets in parallel.

A regulatory ILC (ILCreg) population has previously been described in mice and humans (Wang et al., 2017), through their capacity to produce IL-10. However the characteristics of this population are controversial as other ILC subsets such as ILC2 can also produce IL-10 (Bando et al., 2019). In none of those cases, was the population described to express Foxp3, the hallmark transcription factor for regulatory T cells. The present invention seeks to address one or more of the aforementioned issues.

### SUMMARY OF THE INVENTION

The present invention provides a method for expanding immune cells, the method comprising a) co-culturing the immune cells and at least one epithelial organoid, wherein the epithelial organoid comprises more epithelial cells than mesenchymal cells. The present invention also provides immune cells obtainable by the method of the present invention. Also provided is an in vitro population of innate lymphoid cells (ILCs), wherein the population comprises at least about 5×10³ ILCs. The inventors have also identified a subset of human ILCs called human regulatory ILCs (or ILCregs). These are defined in more detail below and may be used to suppress inflammation, especially in the intestine. The term “organoid” is a known term of the art, which refers to a plurality of cells which self-assemble in vitro to form a complex structure. The organoid is 3D and resembles an in vitro miniaturized version of an organ or section of an organ thereof. Generally, organoids are 3D. Further generally, co-culture is with at least one whole epithelial organoid. However, in some embodiments culture is with a portion of at least one epithelial organoid. A portion may comprise a layer of cells, which will comprise all cell types comprised in the full organoid. In such embodiments, it will be appreciated that the portion may be obtained from mechanically or chemically breaking up the organoid. In the context of the present invention, the term “epithelial organoid” refers to an organoid comprising epithelial cells.

Advantageously, organoids can remain viable and stable in vitro for extended periods of time. For example, organoids may remain viable and stable in vitro for at least two weeks, at least three weeks, at least four weeks, at least five weeks, at least six weeks, at least three months, at least six months, at least nine months, at least 12 months, at least 18 months or at least 24 months. The long-term stability of the organoid, together with its complex structure, provides an in vitro culture environment which closely correlates to the in vivo environment. Such long-term stability also enables research of the organoid over an extended period of time which also more closely correlates to in vivo time periods.

The present inventors have found that the co-culture of at least one epithelial organoid comprising more epithelial cells than mesenchymal cells and immune cells leads to significant expansion of the immune cells. Prior to this finding, it was believed that mesenchymal cells were essential to ensure maintenance and expansion of the immune cells. Therefore, the significant expansion achieved by the present method is entirely unexpected. The inventors have also surprisingly found that the co-culture of at least one epithelial organoid comprising more epithelial cells than mesenchymal cells and immune cell precursors differentiates the immune cell precursors into substantial numbers of immune cells. The generation of such substantial numbers of immune cells may have particular utility in cell therapy, where it currently can be difficult to obtain sufficient numbers of mature immune cells for therapeutic purposes or even for in vitro study.

As the skilled person will appreciate, a mesenchymal cell is a stromal cell. In the context of the present invention, a mesenchymal cell is a cell having plastic adherent properties under normal culture conditions and has a fibroblast-like morphology. Cultured mesenchymal cells may be CD90 and CD105 positive. Cultured mesenchymal cells may be CD90, CD105 and CD73 positive. Cultured mesenchymal cells may be CD73, CD90, CD105, CD44, CD106 and CD166 positive. Cultured mesenchymal cells may be CD11b, CD14, CD19, CD34, CD45, CD79a and HLA-DR negative and CD73, CD90 and CD105 positive. Cultured mesenchymal cells may be CD11b, CD14, CD19, CD34, CD45, CD79a and HLA-DR negative and CD73, CD90, CD105, CD44, CD106 and CD166 positive.

In the context of the present invention, if a cell is defined as positive for a particular marker, for example CD45, it will be appreciated that the cell comprises a detectable level of the marker. Conversely, if a cell is defined as negative for a particular marker, it will be appreciated that the cell comprises an undetectable level of the marker. Methods for measuring the presence of markers/proteins/mRNA are known in the art and discussed in more detail below. In the context of the present invention, the terms “amount” and “level” are interchangeable.

Mesenchymal cells may comprise multipotent mesenchymal cells. Multipotent mesenchymal cells are capable of differentiating into a plurality of different cell types.

Preferably, the mesenchymal cells comprise or consist of fibroblasts.

In the context of the present invention, the term “expansion” refers to the generation or production of immune cells. Thus, in the context of the present invention, the term “expansion” comprises proliferation of the immune cells and/or differentiation of the immune cells into differentiated immune cells. The term “expansion” does not relate to activation of a specific sub-clone, such as a specific T-cell clone.

In some embodiments, the epithelial organoid comprises less than about 45% mesenchymal cells. In some embodiments, the epithelial organoid comprises less than about 44% mesenchymal cells, less than about 43% mesenchymal cells, less than about 42% mesenchymal cells, less than about 41% mesenchymal cells, less than about 40% mesenchymal cells, less than about 39% mesenchymal cells, less than about 38% mesenchymal cells, less than about 37% mesenchymal cells, less than about 36% mesenchymal cells, less than about 35% mesenchymal cells, less than about 34% mesenchymal cells, less than about 33% mesenchymal cells, less than about 32% mesenchymal cells, less than about 31% mesenchymal cells, less than about 30% mesenchymal cells, less than about 29% mesenchymal cells, less than about 28% mesenchymal cells, less than about 27% mesenchymal cells, less than about 26% mesenchymal cells, less than about 25% mesenchymal cells, less than about 24% mesenchymal cells, less than about 23% mesenchymal cells, less than about 22% mesenchymal cells, less than about 21% mesenchymal cells, less than about 20 mesenchymal cells, less than about 19% mesenchymal cells, less than about 18% mesenchymal cells, less than about 17% mesenchymal cells, less than about 16% mesenchymal cells, less than about 15% mesenchymal cells, less than about 14 mesenchymal cells, less than about 13% mesenchymal cells, less than about 12% mesenchymal cells, less than about 11% mesenchymal cells, less than about 10% mesenchymal cells, less than about 9% mesenchymal cells, less than about 8% mesenchymal cells, less than about 7% mesenchymal cells, less than about 6% mesenchymal cells, less than about 5% mesenchymal cells, less than about 4% mesenchymal cells, less than about 3% mesenchymal cells, less than about 2% mesenchymal cells or less than about 1% mesenchymal cells.

In some embodiments, the epithelial organoid comprises less than about 40% mesenchymal cells. In some embodiments, the epithelial organoid comprises less than about 35% mesenchymal cells. In some embodiments, the epithelial organoid comprises less than 30% mesenchymal cells. In some embodiments, the epithelial organoid comprises less than 25% mesenchymal cells. In some embodiments, the epithelial organoid comprises less than 24% mesenchymal cells. In some embodiments, the epithelial organoid comprises less than 23% mesenchymal cells. In some embodiments, the epithelial organoid comprises less than 22% mesenchymal cells. In some embodiments, the epithelial organoid comprises less than 21% mesenchymal cells. In some embodiments, the epithelial organoid comprises less than 20% mesenchymal cells. In some embodiments, the epithelial organoid comprises less than 15% mesenchymal cells. In some embodiments, the epithelial organoid comprises less than 10% mesenchymal cells.

In some embodiments, the epithelial organoid comprises of from about 0.01% mesenchymal cells to about 45% mesenchymal cells. In some embodiments, the epithelial organoid comprises of from about 0.01% mesenchymal cells to about 30% mesenchymal cells. In some embodiments, the epithelial organoid comprises of from about 0.01% mesenchymal cells to about 25% mesenchymal cells. In some embodiments, the epithelial organoid comprises of from about 0.01% mesenchymal cells to about 20% mesenchymal cells. In some embodiments, the epithelial organoid comprises of from about 0.01% mesenchymal cells to about 15% mesenchymal cells. In some embodiments, the epithelial organoid comprises of from about 0.01% mesenchymal cells to about 10% mesenchymal cells.

In some embodiments, the epithelial organoid comprises of from about 0.1% mesenchymal cells to about 45% mesenchymal cells. In some embodiments, the epithelial organoid comprises of from about 0.1% mesenchymal cells to about 30% mesenchymal cells. In some embodiments, the epithelial organoid comprises of from about 0.1% mesenchymal cells to about 25% mesenchymal cells. In some embodiments, the epithelial organoid comprises of from about 0.1% mesenchymal cells to about 20% mesenchymal cells.

In some embodiments, the epithelial organoid comprises of from about 1% mesenchymal cells to about 45% mesenchymal cells. In some embodiments, the epithelial organoid comprises of from about 1% mesenchymal cells to about 30% mesenchymal cells. In some embodiments, the epithelial organoid comprises of from about 1% mesenchymal cells to about 25% mesenchymal cells. In some embodiments, the epithelial organoid comprises of from about 1% mesenchymal cells to about 20% mesenchymal cells.

The epithelial organoid may not comprise a detectable level of mesenchymal cells. Thus, in some embodiments, the epithelial organoid comprises an undetectable level of mesenchymal cells.

Various methods for detecting a percentage or level are known to those skilled in the art. For example, the percentage or level of mesenchymal cells in the organoid may be detected by flow cytometry. Another suitable method of detection may comprise fluorescence microscopy, for example using a confocal microscope. Other suitable methods will be known to the skilled person. Detection of the percentage or level of mesenchymal cells may be prior to step (a). Alternatively, detection of the percentage or level of mesenchymal cells may be during or after step (a).

Before step (a), the method may comprise a step of depleting mesenchymal cells from the epithelial organoid. Alternatively, the epithelial organoid of the invention may have undergone depletion of mesenchymal cells. By “depleting” mesenchymal cells, this will be understood to refer to removal of mesenchymal cells from the epithelial organoid. Thus, “undergone depletion” will refer to an epithelial organoid which has already undergone removal of mesenchymal cells.

Before step (a), the method may comprise a step of depleting mesenchymal cells from the epithelial organoid immediately before step (a) and/or after full maturation of the epithelium.

An epithelial organoid which has undergone depletion of mesenchymal cells may comprise less than about 45% mesenchymal cells, less than about 44% mesenchymal cells, less than about 43% mesenchymal cells, less than about 42% mesenchymal cells, less than about 41% mesenchymal cells, less than about 40% mesenchymal cells, less than about 39% mesenchymal cells, less than about 38% mesenchymal cells, less than about 37% mesenchymal cells, less than about 36% mesenchymal cells, less than about 35% mesenchymal cells, less than about 34% mesenchymal cells, less than about 33% mesenchymal cells, less than about 32% mesenchymal cells, less than about 31% mesenchymal cells, less than about 30% mesenchymal cells, less than about 29% mesenchymal cells, less than about 28% mesenchymal cells, less than about 27% mesenchymal cells, less than about 26% mesenchymal cells, less than about 25% mesenchymal cells, less than about 24% mesenchymal cells, less than about 23% mesenchymal cells, less than about 22% mesenchymal cells, less than about 21% mesenchymal cells, less than about 20 mesenchymal cells, less than about 19% mesenchymal cells, less than about 18% mesenchymal cells, less than about 17% mesenchymal cells, less than about 16% mesenchymal cells, less than about 15% mesenchymal cells, less than about 14 mesenchymal cells, less than about 13% mesenchymal cells, less than about 12% mesenchymal cells, less than about 11% mesenchymal cells, less than about 10% mesenchymal cells, less than about 9% mesenchymal cells, less than about 8% mesenchymal cells, less than about 7% mesenchymal cells, less than about 6% mesenchymal cells, less than about 5% mesenchymal cells, less than about 4% mesenchymal cells, less than about 3% mesenchymal cells, less than about 2% mesenchymal cells or less than about 1% mesenchymal cells, as defined above.

Depletion may comprise mechanical disruption of the epithelial organoid. Advantageously, mechanical disruption separates a mesenchymal fraction (if present) from the epithelial structure of the epithelial organoid. The mesenchymal fraction can then be removed, leaving the epithelial structure of the organoid. In some embodiments, depletion comprises digestion of the epithelial organoid, for example digestion using collagenase.

Depletion may be repeated two or more times. For example, depletion may be repeated three, four, five or six times.

In some embodiments, depletion comprises mechanical disruption of the epithelial organoid which is repeated three, four or five times. In other embodiments, depletion comprises i) mechanical disruption of the epithelial organoid and ii) digestion of the epithelial organoid using collagenase. Preferably, digestion of the epithelial organoid using collagenase is after mechanical disruption.

The epithelial organoid may be a primary organoid or derived from stem cells. The stem cells may comprise or consist of induced pluripotent stem cells (iPSCs) or adult stem cells. As the skilled person will appreciate, iPSCs are a pluripotent stem cell obtained by genetic reprogramming of adult somatic cells into an embryonic state. By primary organoid, this will be understood to refer to an organoid obtained from a subject biopsy sample. The subject biopsy sample may have been obtained during an endoscopy. The subject biopsy sample is preferably a human biopsy sample. The subject biopsy sample is more preferably a human small intestine biopsy sample or a human colon biopsy sample. The subject biopsy sample is most preferably a human small intestine biopsy sample. The organoid is preferably a human small intestine biopsy derived organoid or a human colon biopsy derived organoid. The organoid is most preferably a human small intestine biopsy derived organoid. The effectiveness of such organoids is shown in Example 10.

In embodiments comprising an organoid derived from iPSCs, the iPSCs may be obtained from the Human Induced Pluripotent Stem Cells Initiative (HipSci, https://www.hipsci.org).

Alternatively, in embodiments comprising an organoid derived from iPSCs, the method may comprise a step before step (a) of producing the iPSCs.

Producing the iPSCs may comprise introducing a polynucleotide sequence encoding one or more of OCT3/4, SOX2, KLF4 and MYC into isolated primary cells. Preferably, the polynucleotide sequence encodes OCT3/4, SOX2, KLF4 and MYC. More preferably, the polynucleotide sequence encodes human OCT3/4, human SOX2, human KLF4 and human MYC. Introduction may comprise transduction or transfection, typically transduction. In some embodiments a vector comprises the polynucleotide sequence. The vector may be a Sendai vector. After introduction, the cells may be cultured in an iPS cell medium for at least five, at least ten, at least 20, at least 30 or at least 40 days. Culture may be on a feeder layer. The iPS cell medium may comprise advanced DMEM.

The iPS cell medium may further comprise Knockout Serum Replacement (KSR), which is commercially available from Life Technologies. The iPS cell medium may comprise about 1%, about 2%, about 3%, about 4%, about 5%, about 6%, about 7%, about 8%, about 9%, about 10%, about 15% or about 20% KSR. In some embodiments the iPS cell medium comprises about 10% KSR.

In some embodiments, the iPS cell medium further comprises L-glutamine and/or Fibroblast Growth Factor-2. The iPS cell medium may comprise L-glutamine at a concentration of at least about 0.5 mM, about 1 mM, about 2 mM, about 3 mM, about 4 mM or about 5 mM. The iPS cell medium may comprise less than about 10 mM, less than about 7 mM, less than about 6 mM or less than about 5 mM L-glutamine. In some embodiments the iPS cell medium comprises of from about 1 mM to about 3 mM L-glutamine. In some embodiments the iPS cell medium comprises about 2 mM L-glutamine.

The Fibroblast Growth Factor-2 may be Zebrafish Fibroblast Growth Factor-2. In some embodiments the Fibroblast Growth Factor-2 is recombinant. The iPS cell medium may comprise Fibroblast Growth Factor-2 at a concentration of at least about 1 ng/ml, about 2 ng/ml, about 3 ng/ml, about 4 ng/ml, about 5 ng/ml or at least about 10 ng/ml. In some embodiments the iPS cell medium comprises less than about 20 ng/ml, less than about 10 ng/ml or less than about 5 ng/ml Fibroblast Growth Factor-2. In some embodiments the iPS cell medium comprises about 4 ng/ml Fibroblast Growth Factor-2.

The iPS cell medium may further comprise an antibiotic, for example pen/strep. The iPS cell medium may comprise about 0.1%, about 0.2%, about 0.5%, about 1%, about 2%, about 3%, about 4% or about 5% pen/strep. In some embodiments the iPS cell medium comprises about 1% pen/strep.

The iPS cell medium may further comprise 2-mercaptoethanol. The iPS cell medium may comprise about 0.001%, about 0.002%, about 0.005%, about 0.007%, about 0.01%, about 0.02%, about 0.03%, about 0.04% or about 0.05% 2-mercaptoethanol. In some embodiments the iPS cell medium comprises about 0.007% 2-mercaptoethanol.

Methods for obtaining primary epithelial organoids from subject biopsy samples are well known in the art. In embodiments comprising primary epithelial organoids, the method may comprise a step before step (a) of obtaining the primary epithelial organoid from a subject biopsy sample. In embodiments comprising primary epithelial organoids, the method may comprise a step before step (a) of obtaining the primary epithelial organoid from a human biopsy sample. Obtaining the primary epithelial organoid from a subject biopsy sample may comprise culturing a subject biopsy sample (or portions of the subject biopsy sample thereof) suspension in a matrix and a basal medium. In the context of the present invention, matrix will be understood to refer to a cell culture matrix. Various cell culture matrices are commercially available. Suitable matrices may include, but not necessarily be limited to Geltrex, Cultrex, Matrigel, Collagen I hydrogels, IV hydrogels and other synthetic hydrogels derived from crosslinking of functionalised polypeptides and/or polymers like Polyethylene Glycol (PEG) as described, for example, in Jowett et al. 2021. In some embodiments the matrix comprises or consists of Matrigel.

Alternatively, obtaining the primary epithelial organoid from a subject biopsy sample may comprise culturing a subject biopsy sample (or portions of the subject biopsy sample thereof) suspension in a hanging drop suspension. By “hanging drop suspension”, this will be understood to refer to suspension in a basal medium from a surface. Various hanging drop suspension culture modules are available to those skilled in the art.

In some embodiments, obtaining the primary epithelial organoid from a subject biopsy sample comprises culturing a subject biopsy sample (or portions of the subject biopsy sample thereof) suspension in Matrigel and a basal medium.

The basal medium may comprise Advanced DMEM/F12. Other suitable basal mediums will be known to those skilled in the art.

The basal medium may further comprise L-glutamine, antibiotic, N2 supplement, B27 supplement, HEPES and/or N-acetylcysteine.

The basal medium may comprise L-glutamine at a concentration of at least about 0.5 mM, about 1 mM, about 2 mM, about 3 mM, about 4 mM or about 5 mM. The basal medium may comprise less than about 10 mM, less than about 7 mM, less than about 6 mM or less than about 5 mM L-glutamine. In some embodiments the basal medium comprises of from about 1 mM to about 3 mM L-glutamine. In some embodiments the basal medium comprises about 2 mM L-glutamine.

The basal medium may comprise HEPES at a concentration of at least about 1 mM, about 2 mM, about 3 mM, about 4 mM, about 5 mM, about 6 mM, about 7 mM, about 8 mM, about 9 mM, about 10 mM, about 12 mM, about 15 mM or at least about 20 mM. The basal medium may comprise less than about 50 mM, about 40 mM, about 30 mM, about 20 mM or about 10 mM HEPES. In some embodiments the basal medium comprises HEPES at a concentration of from about 5 mM to about 15 mM. In some embodiment the basal medium comprises about 10 mM HEPES.

The basal medium may comprise N-acetylcysteine at a concentration of at least about 0.5 mM, about 1 mM, about 2 mM, about 3 mM, about 4 mM or about 5 mM. The basal medium may comprise less than about 10 mM, less than about 7 mM, less than about 6 mM or less than about 5 mM N-acetylcysteine. In some embodiments the basal medium comprises of from about 0.5 mM to about 3 mM N-acetylcysteine. In some embodiments the basal medium comprises about 1 mM N-acetylcysteine.

Culturing the subject biopsy sample (or portions of the subject biopsy sample thereof) suspension may be for a period of at least 120 hours, at least 168 hours, at least two weeks, at least three weeks, at least four weeks, at least five weeks, at least six weeks, at least seven weeks or at least eight weeks. In some embodiments, culturing a subject biopsy sample (or portions of the subject biopsy sample thereof) suspension is for a period of from 120 hours to eight weeks.

Preferably, obtaining the primary epithelial organoid from a subject biopsy sample comprises culturing a subject biopsy sample (or portions of the subject biopsy sample thereof) suspension in a matrix and a basal medium for a period of at least 120 hours, at least 168 hours, at least two weeks, at least three weeks, at least four weeks, at least five weeks, at least six weeks, at least seven weeks or at least eight weeks. In some embodiments, obtaining the primary epithelial organoid from a subject biopsy sample comprises culturing a subject biopsy sample (or portions of the subject biopsy sample thereof) suspension in a matrix and a basal medium for a period of from 120 hours to eight weeks.

Preferably, the basal medium comprises Noggin. In some embodiments, the basal medium comprises R-Spondin1. In some embodiments, the basal medium comprises EGF. In some embodiments the basal medium comprises Noggin, R-Spondin1 and EGF. The basal medium may further comprise one or more of FGF10, CHIR and RhoK-inhibitor.

More preferably, the epithelial organoid is derived from iPSCs or adult stem cells. Most preferably, the epithelial organoid is derived from iPSCs. Advantageously, the derivation of an organoid from a stem cell allows the in vitro differentiation of the stem cells into a plurality of different cell types. This plurality of different cell types can self-assemble in vitro into the complex 3D structure of the organoid.

Methods for generating epithelial organoids from iPSCs or adult stem cells are known in the art. Thus, in some embodiments, before step (a) the method comprises an initial step of generating the organoid in vitro from iPSCs or adult stem cells. Preferably, before step (a) the method comprises an initial step of generating the organoid in vitro from iPSCs. Generating the organoid in vitro from iPSCs may comprise culture of the iPSCs in an organoid differentiation medium. In some embodiments, the method comprises:
i) culturing the iPSCs in a plurality of different organoid differentiation media for a total time period of at least about 48 hours to form organoid colonies;
ii) selecting organoid colonies; and optionally
iii) culturing the organoid colonies.

In some embodiments, the iPSCs are human iPSCs.

The organoid differentiation media may comprise RPMI medium or E8 medium supplemented with the appropriate supplements for differentiation.

For example, for the generation of an intestinal organoid, suitable mediums may comprise endoderm differentiation and mid/hindgut differentiation media. An exemplary endoderm differentiation medium may comprise RPMI medium, B27 and Activin A. An exemplary mid/hindgut differentiation medium may comprise RPMI medium, B27, FGF4 and CHIR.

The endoderm or mid/hindgut differentiation medium may comprise at least about 0.1% B27, at least about 0.2% B27, at least about 0.3% B27, at least about 0.4% B27, at least about 0.5% B27, at least about 0.6% B27, at least about 0.7% B27, at least about 0.8% B27, at least about 0.9% B27, at least about 1% B27, at least about 1.5% B27, at least about 2% B27 or at least about 3% B27. In some embodiments the endoderm differentiation medium comprises less than about 10% B27, less than about 5% B27 or less than about 3% B27. In some embodiments the endoderm differentiation medium comprises about 0.2% B27. In other embodiments the endoderm differentiation medium comprises about 1% B27. In some embodiments the mid/hindgut differentiation medium comprises about 2% B27.

The endoderm differentiation medium may comprise at least about 0.5 μl/ml Activin A, at least about 1 μl/ml Activin A, at least about 2 μl/ml Activin A, at least about 3 μl/ml Activin A or at least about 4 μl/ml Activin A. In some embodiments the endoderm differentiation medium comprises less than about 5 μl/ml Activin A. In some embodiments the endoderm differentiation medium comprises about 1 μl/ml Activin A.

The mid/hindgut differentiation medium may comprise at least about 0.5 μl/ml Fibroblast Growth Factor 4 (FGF4), at least about 1 μl/ml FGF4, at least about 2 μl/ml FGF4, at least about 3 μl/ml FGF4 or at least about 4 μl/ml FGF4. In some embodiments the mid/hindgut differentiation medium comprises less than about 5 μl/ml FGF4. In some embodiments the mid/hindgut differentiation medium comprises about 1 μl/ml FGF4.

An exemplary organoid generation method for the generation of a human intestinal organoid, is as follows:
1. Culture the human iPSCs in E8 medium comprising B27 and Activin A, for about 24 hours;
2. Replace the E8 medium with endoderm differentiation medium and culture the human iPSCs in the endoderm differentiation medium for at least about 72 hours;
3. Replace the endoderm differentiation medium with mid/hindgut differentiation medium and culture the human iPSCs in the mid/hindgut differentiation medium for at least about 72 hours.

Alternatively, the method comprises an initial step of generating the organoid in vitro from adult stem cells, preferably murine adult stem cells. Generating the organoid in vitro from adult stem cells may comprise culture of the adult stem cells in an organoid differentiation medium for a period of at least about 4 hours. In some embodiments, the method comprises:
i) culturing the adult stem cells in a organoid differentiation medium for at least about 4 hours to form organoid colonies;
ii) selecting organoid colonies; and optionally
iii) culturing the organoid colonies.

The organoid differentiation media may comprise RPMI medium or E8 medium supplemented with the appropriate supplements for differentiation.

For the generation of an intestinal organoid from adult murine stem cells, the organoid differentiation medium may comprise a basal medium as defined above. For example, the organoid differentiation medium may comprise Advanced DMEM/F12, L-glutamine, antibiotic, N2 supplement, B27 supplement, R-spondin, EGF, Noggin, HEPES and/or N-acetylcysteine. The basal medium may further comprise one or more of FGF10, CHIR and RhoK-inhibitor.

Other suitable organoid generation methods will be known and available to those skilled in the art.

The at least one epithelial organoid may be a plurality of epithelial organoids. For example, the at least one epithelial organoid may be at least two epithelial organoids, at least three epithelial organoids, at least four epithelial organoids, at least five epithelial organoids, at least six epithelial organoids, at least seven epithelial organoids, at least eight epithelial organoids, at least nine epithelial organoids, at least ten epithelial organoids, at least 20 epithelial organoids, at least 30 epithelial organoids, at least 40 epithelial organoids, at least 50 epithelial organoids, at least 60 epithelial organoids, at least 70 epithelial organoids, at least 80 epithelial organoids, at least 90 epithelial organoids, at least 100 epithelial organoids, at least 150 epithelial organoids, at least 200 epithelial organoids or at least 500 epithelial organoids.

In some embodiments, the at least one epithelial organoid is less than 150 epithelial organoids, less than 100 epithelial organoids, less than 90 epithelial organoids, less than 80 epithelial organoids, less than 70 epithelial organoids, less than 60 epithelial organoids, less than 50 epithelial organoids, less than 40 epithelial organoids or less than 30 epithelial organoids.

In some embodiments, the at least one epithelial organoid is of from two organoids to 100 organoids. In some embodiments, the at least one epithelial organoid is of from 25 to 100 epithelial organoids, preferably, of from 25 to 50 epithelial organoids.

It will be appreciated that the numbers of organoids described above may be in relation to one co-culture, for example one co-culture well of a tissue culture plate. Thus, the total numbers of organoids may be much greater when multiple co-culture wells are used and/or when the method is a high throughput method. For example, the total number of organoids may be at least 10, at least 20, at least 30, at least 40, at least 50, at least 60, at least 70, at least 80, at least 90, at least 100, at least 150, at least 200, at least 250, at least 500, at least 1000, at least 10000, at least 50,000, at least 100,000, at least 200,000, at least 300,000, at least 400,000, at least 500,000, at least 1000000 or at least 5000000.

In some embodiments, the total number of organoids is less than 100000000 organoids. In some embodiments the total number of organoids is of from 10 to 100000000 organoids. The total number of organoids may be of from 20 to 500,000 organoids. In some embodiments, the total number of organoids is of from 20 to 10000 organoids, optionally of from 20 to 1000 organoids. In some embodiments, the total number of organoids is of from 20 to 500 organoids. The total number of organoids may be of from 20 to 250 organoids.

Preferably, the epithelial organoid is a human or murine epithelial organoid. More preferably, the epithelial organoid is a human epithelial organoid. In the context of the present invention, the term “murine” will be understood to encompass rat and mouse. Thus, in some embodiments, the epithelial organoid is a murine epithelial organoid, preferably a mouse epithelial organoid.

The epithelial organoid may be a primary human epithelial organoid or derived from human iPSCs or human adult stem cells. Alternatively, the epithelial organoid may be a primary mouse epithelial organoid or derived from mouse iPSCs, mouse embryonic stem cells or mouse adult stem cells. When the epithelial organoid is a primary mouse epithelial organoid, the mouse from which the organoid is isolated may be germ-free or specific pathogen free. The present inventors have found that organoids from such germ-free or specific pathogen free mice are suitable for the present invention. This is surprising given the known role of microorganisms in organ differentiation and maintenance, particularly intestinal differentiation and maintenance.

In embodiments comprising a plurality of epithelial organoids, the plurality of epithelial organoids preferably comprise or consist of epithelial organoids from the same species. For example, the plurality of epithelial organoids may comprise or consist of a plurality of epithelial human organoids. Alternatively, the plurality of epithelial organoids may comprise or consist of a plurality of epithelial murine organoids, for example a plurality of epithelial mouse organoids. Alternatively, the plurality of epithelial organoids may consist of epithelial organoids from at least two different species, for example human and mouse. Optionally, the plurality of epithelial organoids comprise or consist of a plurality of murine and human epithelial organoids.

Since the epithelial organoid is an in vitro miniaturized version of an organ or section of an organ thereof, it will be appreciated that any epithelial organoid can be used in the present invention. In particular, the epithelial organoid can be an in vitro miniaturized version of any organ (or section of an organ thereof) which comprises epithelial cells. For example, the epithelial organoid may be a skin, gastro-intestinal, lung, thymic, thyroid, reproductive, bladder, kidney, pancreas or liver epithelial organoid. In some embodiments, the epithelial organoid is a skin, gastro-intestinal, lung, thyroid, reproductive, bladder, kidney, pancreas, or liver epithelial organoid. In some embodiments, the epithelial organoid is a lung, reproductive or gastro-intestinal epithelial organoid. In some embodiments the epithelial organoid is a lung or gastro-intestinal epithelial organoid.

Reproductive epithelial organoids may comprise fallopian tube epithelial organoids, ovary epithelial organoids, prostate epithelial organoids, endometrium epithelial organoids, cervix epithelial organoids, vaginal epithelial organoids and testes epithelial organoids (which may otherwise be referred to as gonadal epithelial organoids). Cervix epithelial organoids may comprise endocervical canal epithelial organoids and/or ectocervix epithelial organoids. Reproductive epithelial organoids may be selected from fallopian tube epithelial organoids, ovary epithelial organoids, prostate epithelial organoids and endometrium epithelial organoids.

In the context of the present invention, the term “gastro-intestinal” will be understood to refer to oral mucosal organs, stomach, intestine and anus. The term “oral mucosal” will be understood to refer to mucosal organs of the oral tract, such as the salivary gland, pharynx, taste buds, lingual region and oesophagus. Gastro-intestinal epithelial organoids may thus comprise stomach epithelial organoids, salivary gland epithelial organoids, taste bud epithelial organoids, lingual region epithelial organoids, oesophagus epithelial organoids, pharynx epithelial organoids, intestinal epithelial organoids and anal epithelial organoids. In some embodiments the epithelial organoid is an oral mucosal epithelial organoid or an intestinal epithelial organoid. In some embodiments, the epithelial organoid is an oral mucosal epithelial organoid. The oral mucosal epithelial organoid may be selected from a stomach epithelial organoid and oesophageal epithelial organoid.

In some embodiments the epithelial organoid is an intestinal epithelial organoid. It will be appreciated that intestinal organoids comprise small intestinal organoids, large intestinal organoids and rectum organoids. In some embodiments, the intestinal epithelial organoids are small intestinal epithelial organoids. In other embodiments, the intestinal epithelial organoids are large intestinal epithelial organoids. Large intestinal epithelial organoids may comprise or consist of colon epithelial organoids. In some embodiments, the intestinal epithelial organoids are rectum organoids.

Preferably, the epithelial organoid is an intestinal epithelial organoid or lung epithelial organoid. In some embodiments the epithelial organoid is an intestinal epithelial organoid. In some embodiments the epithelial organoid is a small intestinal epithelial organoid or a lung epithelial organoid. Alternatively, the epithelial organoid may be a lung epithelial organoid.

The epithelial organoid may be an epithelial cancer organoid. By epithelial cancer organoid, this will be understood to refer to an organoid obtained from a cancerous tumour biopsy sample. For example, the epithelial cancer organoid may be an epithelial skin cancer, intestinal cancer, lung cancer, thymic cancer, thyroid cancer, reproductive cancer, bladder cancer, kidney cancer, pancreas cancer, oral mucosal cancer or liver cancer organoid. In some embodiments, the epithelial cancer organoid is an epithelial skin cancer, intestinal cancer, lung cancer, thyroid cancer, reproductive cancer, bladder cancer, kidney cancer, pancreas cancer, oral mucosal cancer or liver cancer organoid. The epithelial cancer organoid may be an epithelial head and neck cancer organoid. It will be appreciated that an epithelial head and neck cancer organoid originates from a head and neck cancerous tumour biopsy sample.

In some embodiments, the epithelial organoid is a primary skin, intestinal, lung, thymic, thyroid, reproductive, bladder, kidney, pancreas, oral mucosal or liver epithelial organoid. In some embodiments, the epithelial organoid is a primary skin, intestinal, lung, thyroid, reproductive, bladder, kidney, pancreas, oral mucosal or liver epithelial organoid. In some embodiments, the epithelial organoid is a primary intestinal, lung, reproductive or oral mucosal epithelial organoid. Primary epithelial organoids, as defined above, are organoids comprising epithelial cells obtained from a subject biopsy sample. A primary epithelial intestinal organoid may otherwise be referred to as an enteroid. A primary colon epithelial organoid may otherwise be referred to as a colonoid.

The present inventors have surprisingly found that immune cells obtained by the method of the present invention may be imprinted with a genetic signature and phenotype specific of in vivo immune cells in the primary organ. This advantageously allows the generation of tissue-specific immune cells which may have utility for the treatment of particular diseases and/or may have improved homing capacity to the specific tissue when administered to a subject. When the epithelial organoid is an intestinal organoid, the immune cells obtained may thus have an intestinal-specific genetic signature and phenotype. When the epithelial organoid is a lung organoid, the immune cells obtained may have a lung-specific genetic signature and phenotype. The tissue-specific genetic signatures and phenotypes are discussed in more detail herein.

Preferably, the immune cells and at least one epithelial organoid are co-cultured in a medium which supports the culture of both the immune cells and the epithelial organoid(s).

By “supports the culture” this will be understood to enable growth, maintenance and differentiation, if necessary. The skilled person can design such a medium. The medium preferably comprises a basal medium comprising one or more of R-Spondin, Noggin, EGF, 2-mercaptoethanol, IL-2, and IL-7. Suitable basal mediums include, but are not necessarily limited to Advanced DMEM/F12 and Essential 8™ medium. Further suitable basal media are known to those skilled in the art. In some embodiments the medium comprises a basal medium comprising R-Spondin, Noggin, EGF, IL-2, and IL-7. In some embodiments the medium comprises a basal medium comprising R-Spondin, Noggin, EGF, 2-mercaptoethanol, IL-2, and IL-7. In some embodiments the medium comprises a basal medium comprising IL-2 and IL-7. In some embodiments the medium comprises a basal medium comprising IL-2, IL-7 and 2-mercaptoethanol. In some embodiments the medium comprises Advanced DMEM/F12 medium comprising R-Spondin, Noggin, EGF, IL-2, and IL-7. In other embodiments the medium comprises Essential 8™ medium comprising R-Spondin, Noggin, EGF, IL-2, and IL-7. In some embodiments the basal medium further comprises IL-23. In some embodiments the basal medium further comprises IL-15. In some embodiments the basal medium further comprises IL-22. In some embodiments the basal medium further comprises TGF-β.

In some embodiments the basal medium comprises IL-2, IL-7, IL-15 and 2-mercaptoethanol. In some embodiments the basal medium comprises R-Spondin, Noggin, EGF, IL-2, IL-7, IL-15 and β-mercaptoethanol. In some embodiments the basal medium comprises Advanced DMEM/F12 medium comprising R-Spondin, Noggin, EGF, IL-2, IL-7, IL-15 and β-mercaptoethanol.

The basal medium may comprise EGF at a concentration of at least about 1 ng/ml, at least about 10 ng/ml, at least about 20 ng/ml, at least about 25 ng/ml, at least about 30 ng/ml, at least about 35 ng/ml, at least about 40 ng/ml, at least about 45 ng/ml, at least about 50 ng/ml, at least about 55 ng/ml, at least about 60 ng/ml, at least about 65 ng/ml, at least about 70 ng/ml, at least about 75 ng/ml, at least about 80 ng/ml, at least about 85 ng/ml, at least about 90 ng/ml, at least about 95 ng/ml or at least about 100 ng/ml. In some embodiments the basal medium comprises EGF at a concentration of at least about 10 ng/ml. In some embodiments the basal medium comprises EGF at a concentration of at least about 25 ng/ml, optionally at least about 50 ng/ml.

The basal medium may comprise EGF at a concentration of less than about 1000 ng/ml, less than about 900 ng/ml, less than about 800 ng/ml, less than about 700 ng/ml, less than about 600 ng/ml, less than about 500 ng/ml, less than about 400 ng/ml, less than about 300 ng/ml, less than about 200 ng/ml, less than about 100 ng/ml or less than about 50 ng/ml. In some embodiments the basal medium comprises EGF at a concentration of less than about 50 ng/ml.

In some embodiments the basal medium comprises EGF at a concentration of from about 1 ng/ml to about 1000 ng/ml. The basal medium may comprise EGF at a concentration of from about 10 ng/ml to about 500 ng/ml, optionally at a concentration of from about 10 ng/ml to about 100 ng/ml. In some embodiments the basal medium comprises EGF at a concentration of about 50 ng/ml.

The EGF may be recombinant. In some embodiments, the EGF is murine or human.

The basal medium may comprise R-spondin at a concentration of at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 ng/ml, at least about 900 ng/ml, at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml or at least about 20 μg/ml. In some embodiments the basal medium comprises R-spondin at a concentration of at least about 500 ng/ml. In some embodiments the basal medium comprises R-spondin at a concentration of at least about 700 ng/ml, optionally at least about 900 ng/ml.

The basal medium may comprise R-spondin at a concentration of less than about 25 μg/ml, less than about 24 μg/ml, less than about 23 μg/ml, less than about 22 μg/ml or less than about 21 μg/ml. In some embodiments the basal medium comprises R-spondin at a concentration of less than about 10 μg/ml.

In some embodiments the basal medium comprises R-spondin at a concentration of from about 100 ng/ml to about 20 μg/ml. The basal medium may comprise R-spondin at a concentration of from about 200 ng/ml to about 10 μg/ml, optionally at a concentration of from about 500 ng/ml to about 2 μg/ml. In some embodiments the basal medium comprises R-spondin at a concentration of about 1 μg/ml.

The R-spondin may be recombinant. In some embodiments, the R-spondin is murine or human. In some embodiments, the R-spondin is an R-spondin-comprising supernatant. The supernatant may have been isolated from an R-spondin-producing cell line, of which various cell lines are available.

The basal medium may comprise Noggin at a concentration of at least about 1 ng/ml, at least about 2 ng/ml, at least about 3 ng/ml, at least about 4 ng/ml, at least about 5 ng/ml, at least about 6 ng/ml, at least about 7 ng/ml, at least about 8 ng/ml, at least about 9 ng/ml, at least about 10 ng/ml, at least about 11 ng/ml, at least about 12 ng/ml, at least about 13 ng/ml, at least about 14 ng/ml, at least about 15 ng/ml, at least about 16 ng/ml, at least about 17 ng/ml, at least about 18 ng/ml, at least about 19 ng/ml, at least about 20 ng/ml, at least about 21 ng/ml, at least about 22 ng/ml, at least about 23 ng/ml, at least about 24 ng/ml, at least about 25 ng/ml, at least about 26 ng/ml, at least about 27 ng/ml, at least about 28 ng/ml, at least about 29 ng/ml, at least about 30 ng/ml, at least about 31 ng/ml, at least about 32 ng/ml, at least about 33 ng/ml, at least about 34 ng/ml, at least about 35 ng/ml, at least about 36 ng/ml, at least about 37 ng/ml, at least about 38 ng/ml, at least about 39 ng/ml, at least about 40 ng/ml, at least about 41 ng/ml, at least about 42 ng/ml, at least about 43 ng/ml, at least about 44 ng/ml, at least about 45 ng/ml, at least about 46 ng/ml, at least about 47 ng/ml, at least about 48 ng/ml, at least about 49 ng/ml or at least about 50 ng/ml. In some embodiments the basal medium comprises Noggin at a concentration of at least about 20 ng/ml. In some embodiments the basal medium comprises Noggin at a concentration of at least about 50 ng/ml, optionally at least about 70 ng/ml.

The basal medium may comprise Noggin at a concentration of less than about 1000 ng/ml, less than about 900 ng/ml, less than about 800 ng/ml, less than about 700 ng/ml, less than about 600 ng/ml, less than about 500 ng/ml, less than about 400 ng/ml, less than about 300 ng/ml, less than about 200 ng/ml, less than about 100 ng/ml or less than about 50 ng/ml. In some embodiments the basal medium comprises Noggin at a concentration of less than about 200 ng/ml.

In some embodiments the basal medium comprises Noggin at a concentration of from about 1 ng/ml to about 1000 ng/ml. The basal medium may comprise Noggin at a concentration of from about 10 ng/ml to about 500 ng/ml, optionally at a concentration of from about 10 ng/ml to about 200 ng/ml. In some embodiments the basal medium comprises Noggin at a concentration of from about 50 ng/ml to about 200 ng/ml. In some embodiments the basal medium comprises Noggin at a concentration of about 100 ng/ml.

The Noggin may be recombinant. In some embodiments, the Noggin is murine or human. In some embodiments, the Noggin is a Noggin-comprising supernatant. The supernatant may have been isolated from a Noggin-producing cell line, of which various cell lines are available.

The basal medium may comprise IL-2 at a concentration of at least about 1 ng/ml, at least about 2 ng/ml, at least about 3 ng/ml, at least about 4 ng/ml, at least about 5 ng/ml, at least about 6 ng/ml, at least about 7 ng/ml, at least about 8 ng/ml, at least about 9 ng/ml, at least about 10 ng/ml, at least about 11 ng/ml, at least about 12 ng/ml, at least about 13 ng/ml, at least about 14 ng/ml, at least about 15 ng/ml, at least about 16 ng/ml, at least about 17 ng/ml, at least about 18 ng/ml, at least about 19 ng/ml, at least about 20 ng/ml, at least about 21 ng/ml, at least about 22 ng/ml, at least about 23 ng/ml, at least about 24 ng/ml, at least about 25 ng/ml, at least about 26 ng/ml, at least about 27 ng/ml, at least about 28 ng/ml, at least about 29 ng/ml, at least about 30 ng/ml, at least about 31 ng/ml, at least about 32 ng/ml, at least about 33 ng/ml, at least about 34 ng/ml, at least about 35 ng/ml, at least about 36 ng/ml, at least about 37 ng/ml, at least about 38 ng/ml, at least about 39 ng/ml, at least about 40 ng/ml, at least about 41 ng/ml, at least about 42 ng/ml, at least about 43 ng/ml, at least about 44 ng/ml, at least about 45 ng/ml, at least about 46 ng/ml, at least about 47 ng/ml, at least about 48 ng/ml, at least about 49 ng/ml or at least about 50 ng/ml. In some embodiments the basal medium comprises IL-2 at a concentration of at least about 10 ng/ml. In some embodiments the basal medium comprises IL-2 at a concentration of at least about 15 ng/ml, optionally at least about 20 ng/ml.

The basal medium may comprise IL-2 at a concentration of less than about 1000 ng/ml, less than about 900 ng/ml, less than about 800 ng/ml, less than about 700 ng/ml, less than about 600 ng/ml, less than about 500 ng/ml, less than about 400 ng/ml, less than about 300 ng/ml, less than about 200 ng/ml, less than about 100 ng/ml or less than about 50 ng/ml. In some embodiments the basal medium comprises IL-2 at a concentration of less than about 50 ng/ml.

In some embodiments the basal medium comprises IL-2 at a concentration of from about 1 ng/ml to about 1000 ng/ml. The basal medium may comprise IL-2 at a concentration of from about 10 ng/ml to about 500 ng/ml, optionally at a concentration of from about 10 ng/ml to about 100 ng/ml. In some embodiments the basal medium comprises IL-2 at a concentration of from about 10 ng/ml to about 50 ng/ml. In some embodiments the basal medium comprises IL-2 at a concentration of about 20 ng/ml.

Preferably, the IL-2 is recombinant. In some embodiments, the IL-2 is murine or human. More preferably, the IL-2 is recombinant human IL-2.

The basal medium may comprise IL-7 at a concentration of at least about 1 ng/ml, at least about 2 ng/ml, at least about 3 ng/ml, at least about 4 ng/ml, at least about 5 ng/ml, at least about 6 ng/ml, at least about 7 ng/ml, at least about 8 ng/ml, at least about 9 ng/ml, at least about 10 ng/ml, at least about 11 ng/ml, at least about 12 ng/ml, at least about 13 ng/ml, at least about 14 ng/ml, at least about 15 ng/ml, at least about 16 ng/ml, at least about 17 ng/ml, at least about 18 ng/ml, at least about 19 ng/ml, at least about 20 ng/ml, at least about 21 ng/ml, at least about 22 ng/ml, at least about 23 ng/ml, at least about 24 ng/ml, at least about 25 ng/ml, at least about 26 ng/ml, at least about 27 ng/ml, at least about 28 ng/ml, at least about 29 ng/ml, at least about 30 ng/ml, at least about 31 ng/ml, at least about 32 ng/ml, at least about 33 ng/ml, at least about 34 ng/ml, at least about 35 ng/ml, at least about 36 ng/ml, at least about 37 ng/ml, at least about 38 ng/ml, at least about 39 ng/ml, at least about 40 ng/ml, at least about 41 ng/ml, at least about 42 ng/ml, at least about 43 ng/ml, at least about 44 ng/ml, at least about 45 ng/ml, at least about 46 ng/ml, at least about 47 ng/ml, at least about 48 ng/ml, at least about 49 ng/ml or at least about 50 ng/ml. In some embodiments the basal medium comprises IL-7 at a concentration of at least about 10 ng/ml. In some embodiments the basal medium comprises IL-7 at a concentration of at least about 15 ng/ml, optionally at least about 20 ng/ml.

The basal medium may comprise IL-7 at a concentration of less than about 1000 ng/ml, less than about 900 ng/ml, less than about 800 ng/ml, less than about 700 ng/ml, less than about 600 ng/ml, less than about 500 ng/ml, less than about 400 ng/ml, less than about 300 ng/ml, less than about 200 ng/ml, less than about 100 ng/ml or less than about 50 ng/ml. In some embodiments the basal medium comprises IL-7 at a concentration of less than about 50 ng/ml.

In some embodiments the basal medium comprises IL-7 at a concentration of from about 1 ng/ml to about 1000 ng/ml. The basal medium may comprise IL-7 at a concentration of from about 10 ng/ml to about 500 ng/ml, optionally at a concentration of from about 10 ng/ml to about 100 ng/ml. In some embodiments the basal medium comprises IL-7 at a concentration of from about 10 ng/ml to about 50 ng/ml. In some embodiments the basal medium comprises IL-7 at a concentration of about 20 ng/ml.

Preferably, the IL-7 is recombinant. In some embodiments, the IL-7 is murine or human. More preferably, the IL-7 is recombinant murine IL-7.

Preferably, the basal medium comprises R-Spondin at a concentration of about 1 μg/ml, Noggin at a concentration of about 100 ng/ml, EGF at a concentration of about 50 ng/ml, IL-2 at a concentration of about 20 ng/ml and IL-7 at a concentration of about 20 ng/ml.

The R-spondin may be recombinant. In some embodiments, the R-spondin is murine or human. In some embodiments, the R-spondin is a R-spondin-comprising supernatant. The supernatant may have been isolated from a R-spondin-producing cell line, of which various cell lines are available.

In some embodiments the basal medium comprises IL-15 at a concentration of at least about 0.1 ng/ml, at least about 0.2 ng/ml, at least about 0.3 ng/ml, at least about 0.4 ng/ml, at least about 0.5 ng/ml, at least about 0.6 ng/ml, at least about 0.7 ng/ml, at least about 0.8 ng/ml, at least about 0.9 ng/ml, at least about 1 ng/ml, at least about 1.1 ng/ml, at least about 1.2 ng/ml, at least about 1.3 ng/ml, at least about 1.4 ng/ml, at least about 1.5 ng/ml, at least about 1.6 ng/ml, at least about 1.7 ng/ml, at least about 1.8 ng/ml, at least about 1.9 ng/ml, at least about 2 ng/ml, at least about 3 ng/ml, at least about 4 ng/ml, at least about 5 ng/ml, at least about 6 ng/ml, at least about 7 ng/ml, at least about 8 ng/ml, at least about 9 ng/ml or at least about 10 ng/ml. The basal medium may comprise IL-15 at a concentration of less than about 20 ng/ml, less than about 15 ng/ml, less than about 12 ng/ml, less than about 10 ng/ml or less than about 5 ng/ml. In some embodiments the basal medium comprises IL-15 at a concentration of from about 0.1 ng/ml to about 5 ng/ml. In some embodiments the basal medium comprises IL-15 at a concentration of from about 0.5 ng/ml to about 2 ng/ml. In some embodiments the basal medium comprises IL-15 at a concentration of about 1 ng/ml. The IL-15 may be recombinant.

In some embodiments the basal medium comprises 2-mercaptoethanol. The basal medium may comprise at least 1 mM, at least 2 mM, at least 3 mM, at least 4 mM, at least 5 mM, at least 6 mM, at least 7 mM, at least 8 mM, at least 9 mM, at least 10 mM, at least 11 mM, at least 12 mM, at least 13 mM, at least 14 mM, at least 15 mM, at least 16 mM, at least 17 mM, at least 18 mM, at least 19 mM, at least 20 mM, at least 21 mM, at least 22 mM, at least 23 mM, at least 24 mM, at least 25 mM, at least 26 mM, at least 27 mM, at least 28 mM, at least 29 mM, at least 30 mM, at least 31 mM, at least 32 mM, at least 33 mM, at least 34 mM, at least 35 mM, at least 36 mM, at least 37 mM, at least 38 mM, at least 39 mM, at least 40 mM, at least 41 mM, at least 42 mM, at least 43 mM, at least 44 mM, at least 45 mM, at least 46 mM, at least 47 mM, at least 48 mM, at least 49 mM, at least 50 mM, at least 51 mM, at least 52 mM, at least 53 mM, at least 54 mM, at least 55 mM, at least 56 mM, at least 57 mM, at least 58 mM, at least 59 mM, at least 60 mM, at least 70 mM, at least 80 mM, at least 90 mM or at least 100 mM 2-mercaptoethanol.

In some embodiments the basal medium comprises no more than 500 mM, no more than 400 mM, no more than 300 mM, no more than 200 mM, no more than 100 mM, no more than 90 mM, no more than 80 mM, no more than 70 mM or no more than 60 mM 2-mercaptoethanol.

In some embodiments the basal medium comprises of from 1 mM to 500 mM 2-mercaptoethanol. In some embodiments the basal medium comprises of from 10 mM to 100 mM 2-mercaptoethanol. Optionally the basal medium comprises about 50 mM 2-mercaptoethanol.

Preferably, the basal medium comprises R-Spondin at a concentration of about 1 μg/ml, Noggin at a concentration of about 100 ng/ml, EGF at a concentration of about 50 ng/ml, IL-2 at a concentration of about 20 ng/ml, IL-7 at a concentration of about 20 ng/ml, IL-15 at a concentration of about 1 ng/ml and 20 μm 2-mercaptoethanol.

In some embodiments the medium/basal medium does not comprise a detectable level of one or more of IL-23, IL-1B, IL-15, IL-12, IL-18, IL-25, and IL-33. In the context of the present invention, the medium/basal medium not comprising a detectable level of a particular marker refers to the marker not being exogenously added or included in the medium. Thus, the medium/basal medium not comprising a detectable level of the marker is in relation to the medium at the start of the method, and does not exclude detectable levels of the marker produced by the immune cells and/or epithelial organoid into the medium during the method. In some embodiments the medium/basal medium does not comprise a detectable level of IL-15, IL-12 and IL-18. In some embodiments the medium/basal medium does not comprise a detectable level of IL-1B, IL-15, IL-12, IL-18 and IL-25. In some embodiments the medium/basal medium does not comprise a detectable level of IL-1B, IL-15, IL-12, IL-18, IL-25 and IL-33. In some embodiments the medium/basal medium does not comprise a detectable level of IL-23, IL-1B, IL-15, IL-12, IL-18 and IL-25. In some embodiments the medium/basal medium does not comprise a detectable level of IL-23, IL-1B, IL-15, IL-12, IL-18, IL-25, and IL-33.

Preferably, the medium is germ-free or specific pathogen free. Most preferably, the medium is medium A as disclosed in the Examples.

Preferably, the immune cells and the at least one epithelial organoid are co-cultured in a matrix. The matrix may comprise or consist of Geltrex, Cultrex or Matrigel. Other commercially available matrices, particularly synthetic hydrogels, are known to the skilled person. In some embodiments the matrix comprises or consists of Matrigel. More preferably, the immune cells and the at least one epithelial organoid are co-cultured in Matrigel in a medium as defined above, preferably a basal medium.

In some embodiments, the immune cells and the at least one epithelial organoid are co-cultured in a transwell. Typically, in such embodiments, a permeable insert separates cell populations. Thus, in such embodiments, the immune cells may be separated from the at least one epithelial organoid, preferably by a permeable insert.

In some embodiments, at least 10%, at least 20%, at least 30%, at least 40%, at least 50%, at least 60%, at least 70%, at least 80%, at least 90% or at least 99% of the medium is replaced of from about every 24 hours to about every 72 hours.

In some embodiments, at least 10%, at least 20%, at least 30%, at least 40%, at least 50%, at least 60%, at least 70%, at least 80%, at least 90% or at least 99% of the medium is replaced about every 24 hours. In some embodiments, at least 10%, at least 20%, at least 30%, at least 40%, at least 50%, at least 60%, at least 70%, at least 80%, at least 90% or at least 99% of the medium is replaced about every 48 hours. In some embodiments, at least 10%, at least 20%, at least 30%, at least 40%, at least 50%, at least 60%, at least 70%, at least 80%, at least 90% or at least 99% of the medium is replaced about every 72 hours.

Preferably, of from about 10% to about 70% of the medium is replaced of from about every 24 hours to about every 72 hours. More preferably, about 50% of the medium is replaced of from about every 24 hours to about every 72 hours. This allows conditioned medium to remain with the cells while supplementing with fresh growth factors.

Preferably, the immune cells and the at least one epithelial organoid are co-cultured at a temperature of at least about 20° C., at least about 25° C., at least about 30° C. or at least about 35° C. More preferably, the immune cells and the at least one epithelial organoid are co-cultured at a temperature of about 37° C.

In some embodiments the immune cells and at least one epithelial organoid are co-cultured in at least about 1%, at least about 2%, at least about 3%, at least about 4%, at least about 5%, at least about 6%, at least about 7%, at least about 8%, at least about 9%, at least about 10% or at least about 20% CO₂. Preferably, the immune cells and at least one epithelial organoid are co-cultured in of from about 1% to about 10% CO₂. More preferably, the immune cells and at least one epithelial organoid are co-cultured in about 5% CO₂.

In some embodiments, the epithelial organoid comprises a metabolite, for example a bacterial metabolite such as succinate or butyrate. For example, the epithelial organoid may be injected with a metabolite, preferably succinate.

In some embodiments, the immune cells are human immune cells, equine immune cells, feline immune cells, canine immune cells, bovine immune cells, ovine immune cells or murine immune cells. In some embodiments, the immune cells are human immune cells or murine immune cells. Optionally, the murine immune cells are mouse immune cells. In some embodiments, the immune cells are human immune cells.

Preferably, the immune cells are primary immune cells. More preferably, the immune cells are primary human cells. By “primary cell” this will be understood to refer to a cell that has been obtained from a subject. Primary cells are not immortalised cells from a cell line.

The primary immune cells may be autologous. Alternatively, the primary immune cells may be allogeneic. In some embodiments, the primary immune cells comprise a mixture of allogeneic and autologous immune cells.

As the skilled person will appreciate, autologous cells are cells from the same subject, i.e. cells which have been obtained from a subject which will be administered back to the same subject. Allogeneic cells are cells obtained from a different subject to the subject to which the cells will be administered. The different subjects are typically from the same species. Allogenic cells are thus genetically different to the subject to which they are administered.

Alternatively, the immune cells may comprise or consist of immortalised immune cells from a cell line.

In some embodiments the immune cells are primary immune cells and the epithelial organoid is a primary epithelial organoid. In some embodiments, the immune cells are primary immune cells and the epithelial organoid is derived from iPSCs or adult stem cells. In some embodiments, the immune cells are primary human immune cells and the epithelial organoid is a primary human epithelial organoid. In some embodiments, the immune cells are primary human immune cells and the epithelial organoid is derived from human iPSCs or human adult stem cells.

In some embodiments the immune cells are primary mouse immune cells and the epithelial organoid is a primary human epithelial organoid. In some embodiments the immune cells are primary mouse immune cells and the epithelial organoid is derived from human iPSCs or human adult stem cells.

In some embodiments the immune cells are primary human immune cells and the epithelial organoid is a primary mouse epithelial organoid. Alternatively, the immune cells may be primary human immune cells and the epithelial organoid may be derived from mouse iPSCs or mouse adult stem cells.

In some embodiments the immune cells are not mouse immune cells and the epithelial organoid is not a mouse epithelial organoid.

Any immune cell is suitable for expansion in the method of the present invention. However, the method of the present invention has particular utility for the expansion of immune cells which may otherwise be difficult to obtain in large numbers, or numbers great enough to be used in cell therapy. The method of the present invention also has utility in the production and expansion of immune cells from progenitor cells, such as haematopoietic stem cells and lymphoid precursor cells. This advantageously enables the production of cells which may otherwise be difficult to obtain ex vivo, and allows real-time studies of the differentiation stages. In addition, the method of the present invention can produce various immune cell types from one type of progenitor cell. This effectively makes the method a “one-stop shop” for the production and expansion of numerous different immune cells reducing cost, complexity and time. Thus, in some embodiments the immune cells are haematopoietic stem cells and/or lymphoid precursor cells.

As the skilled person will appreciate, haematopoietic stem cells are stem cells which are capable of differentiating into myeloid and lymphoid cells. Myeloid cells include, but are not necessarily limited to monocytes, macrophages, neutrophils, basophils, eosinophils, erythrocytes and megakaryocytes. Lymphoid cells comprise T cells, B cells and innate lymphoid cells (ILCs). Therefore, the co-culture of haematopoietic stem cells removes the need for the separate co-culture of mature myeloid or lymphoid cells, since mature myeloid and lymphoid cells can all be generated in vitro from the haematopoietic stem cells. Lymphoid precursor cells may otherwise be referred to as lymphoid progenitor cells or thymocytes. As the skilled person will also appreciate, lymphoid precursor cells develop from haematopoietic stem cells and are a precursor cell type to mature lymphoid cells.

Thus, in some embodiments, the immune cells comprise a mixture of haematopoietic stem cells and lymphoid precursor cells.

In some embodiments, the immune cells are lymphoid and/or myeloid cells. In some embodiments, the immune cells are myeloid cells. For example, the immune cells may be selected from one or more of the following cell types: monocytes, macrophages, neutrophils, basophils, eosinophils, erythrocytes and megakaryocytes. In some embodiments the immune cells do not comprise macrophages.

In some embodiments the immune cells are monocytes, neutrophils, basophils, eosinophils, erythrocytes, megakaryocytes, T-cells, B-cells or innate lymphoid cells (ILC). In some embodiments the immune cells are neutrophils, basophils, eosinophils, erythrocytes, megakaryocytes, T-cells, B-cells or innate lymphoid cells (ILC). In some embodiments the immune cells are neutrophils, basophils, eosinophils, T-cells, B-cells or innate lymphoid cells (ILC).

In some embodiments, the immune cells are lymphoid cells. For example, the immune cells may be T-cells, B-cells and/or innate lymphoid cells (ILC). The immune cells may be primary human lymphoid cells, for example primary human T-cells, B-cells and/or ILCs. In some embodiments, the immune cells comprise T-cells and/or B-cells. In some embodiments, the immune cells comprise T-cells. The T-cells may comprise or consist of αβ T-cells. Alternatively, the T-cells may comprise or consist of γδ T-cells. The T-cells may comprise or consist of Natural Killer T (NKT) cells. In some embodiments, the T-cells are CD4⁺ T cells. In other embodiments, the T-cells are CD8⁺ T-cells. In some embodiments the T-cells comprise CD4⁺ T cells and CD8⁺ T-cells.

In other embodiments, the immune cells comprise or are ILCs. As the skilled person will appreciate, ILCs are innate counterparts of T cells and are capable of expressing cytokines at a detectable level. ILCs are typically found at mucosal barriers in vivo and can be identified by the skilled person.

In some embodiments, the immune cells comprise or consist of human or murine ILCs.

In the context of the present invention, ILCs can be categorised into three groups: Group 1, Group 2 and Group 3 ILCs. Group 1 ILCs may comprise NK cells and ILC1 cells. The NK cells are preferably cytotoxic NK cells. Group 2 ILCs may comprise ILC2 cells, while group 3 may comprise ILC3 and Lymphoid tissue inducer (LTi) cells. Group 2 and Group 3 ILCs typically express CD127. Human regulatory ILCs (ILCregs) are a subset of ILCs identified by the inventors and these are defined in more detail below.

Each subset of ILCs can be identified using various methods. Each subset of ILCs may be identified using flow cytometry and/or RNA sequencing to determine the expression profile.

Murine group 1 ILCs may comprise a Lineage⁻, NK1.1⁺ expression profile. Murine group 1 ILCs may further comprise a NKp46⁺ or NKp46 expression profile. In some embodiments murine group 1 ILCS comprise a NKp46⁺ expression profile. Murine group 1 ILCs may further comprise a T-bet⁺ expression profile. In some embodiments, Group 1 ILCs comprise a Lineage⁻, RORγt⁻ Klrg1⁻ CRTh2⁻ NK1.1⁺ NKp46⁺ expression profile. In some embodiments, Group 1 ILCs may further comprise a Lineage⁻, Klrg1⁻, NK1.1⁺, NKp46⁺ expression profile. By “expression profile” when a cell is positive for a particular marker, this will be understood to mean that the marker is detectable in or on the cell. When a cell is negative for a particular marker, this will be understood to mean that the marker is undetectable in the cell. Expression may be determined by measuring mRNA or protein expression levels.

Murine group 1 ILCs may further comprise one or more of the following expression profiles: CD49a⁺, CD49b⁺, CXCR6⁺, CD200r1⁺ and Ly49 family of receptors⁺.

Human group 1 ILCs may comprise a Lin⁻, RORγt⁻, CD127^{+/-}, CD56^{+/-}, CD161^{+/-} expression profile.

Human group 1 ILCs preferably express detectable levels of one or more, such as 2, 3, 4, 5, 6, 7, 8, 9, 10, 11, 12, 13, 14, 15, 16, 17, 18, 19, 20, 21, 22, 23, 24, 25, 26, 27 or 28, of the following genes: AOAH, CCL3, CCL4, CCL5, CD244, CD247, CST7, EOMES, FCGR3A, FGR, GNLY, GZMB, GZMK, IFNG, IKZF3, IL12RB2, ITGAX, ITGB2, KLRC1, KLRD1, NCAM1, NCR1, NKG7, PRF1, SAMD3, TBX21, TIGIT and ZNF683. Human group 1 ILCs preferably express detectable levels of all these genes. Gene expression is typically measured by measuring messenger RNA (mRNA) expression, for instance using RNA sequencing or single cell RNAseq (scRNAseq) as shown in Example 8. As explained above, human group 1 ILCs preferably comprise human NK cells and/or human ILC1 cells.

Murine group 2 ILCs may comprise a Lineage⁻, CD127⁺, RORγt⁻, GATA-3⁺, Klrg1⁺ ILC2⁺ expression profile. Murine group 2 ILCs may comprise a Lineage⁻, GATA-3⁺, ST2⁺ ILC2⁺ expression profile. Alternatively, murine Group 2 ILCs may comprise a Lineage⁻, GATA-3⁺, ICOS⁺ ILC2⁺ expression profile.

Human group 2 ILCs may comprise a Lineage⁻, RORγt⁻, CD127⁺, GATA-3⁺, CRTH2⁺, c-Kit^{+/-} expression profile. Alternatively, human group 2 ILCs may comprise a Lineage⁻, GATA-3⁺, CRTH2⁺, c-Kit^{+/-}, ST2⁺ expression profile.

Human group 2 ILCs preferably express detectable levels of one or more, such as 2, 3, 4, 5, 6, 7, 8, 9, 10, 11, 12, 13, 14, 15, 16, 17, 18, 19 or 20, of the following genes: ANXA1, BCL11B, CCR2, GATA3, HPGD, HPGDS, IL10RA, IL13, IL17RB, IL32, IL5, IL9R, KLRG1, LGALS1, MAF, MBOAT2, PPARG, PTGDR2, PTGER2 and TNFSF10. Human group 2 ILCs preferably express detectable levels of all of these genes. Gene expression is typically measured by measuring messenger RNA (mRNA) expression, for instance using RNA sequencing or single cell RNAseq (scRNAseq) as shown in Example 8. Murine group 3 ILCs may comprise a Lineage⁻, CD127⁺, RORγt⁺, NK1.1^{+/-}, NKp46^{+/-}, CCR6^{+/}, CD4^{+/-} expression profile.

Human group 3 ILCs may comprise a Lineage⁻, CD127⁺, RORγt⁺, NKp44^{+/-}, c-Kit^{+/-}, CCR6^{+/-} HLA-DR^{+/-} expression profile.

Human group 3 ILCs preferably express detectable levels of one or more, such as 2, 3, 4, 5, 6, 7, 8, 9, 10, 11, 12, 13, 14, 15, 16, 17, 18, 19, 20, 21 or 22, of the following genes: AHR, BCL2A1, CCL20, CD81, CSF2, CXCL8, ID2, IKZF2, IL1R1, IL23R, IL4I1, IRF4, KIT, LIF, LTA4H, NCR1, PECAM1, RBPJ, RORC, TNFRSF25, TNFSF4 and TOX2. Human group 3 ILCs preferably express detectable levels of all of these genes. Gene expression is typically measured by measuring messenger RNA (mRNA) expression, for instance using RNA sequencing or single cell RNAseq (scRNAseq) as shown in Example 8.

“Lineage⁻”, as used herein in relation to mouse ILCs will be understood to refer to a CD3⁻ CD19⁻ Ly6G⁻ expression profile. “Lineage⁻”, as used herein in relation to human ILCs will be understood to refer to a CD3⁻ CD20⁻ CD14⁻ CD19⁻ expression profile.

The invention also provides a human regulatory ILC (ILCreg) which expresses a detectable level of FOXP3. The human ILCreg preferably further expresses detectable levels of one or more, such as 2, 3, 4, 5, 6, 7, 8, 9, 10, 11, 12, 13, 14, 15, 16, or 17, of the following genes: CCR5, CTLA4, FGGY, GATA3, GZMB, IL10, IL1R1, IL2RA, IL2RB, KAT2B, LGLS3, PIM1, PRDM1, RUNX1, SOX4, TNFRSF18 and TRAF1. The human ILCreg preferably expresses detectable levels of all of these genes. Gene expression is typically measured by measuring messenger RNA (mRNA) expression, for instance using RNA sequencing or single cell RNAseq (scRNAseq). It is worthy of note FOXP3, RUNX1, IL1R1 and CTLA4 are associated with human T-regulatory cells (Tregs). The human ILCreg preferably further secretes a detectable level of IL-10. This can be measured using a standard cytokine release assay. The human ILCreg preferably expresses detectable levels of one or more, such as 2 or 3, of the following cell surface markers: CD25 (IL2RA), CD127 and CTLA4. The human ILCreg preferably expresses detectable levels of CD25 (IL2RA), CD127, CTLA4, CD25 and CD127, CD25 and CTLA4, CD127 and CTLA4, or CD25, CD127 and CTLA4. Surface marker expression can be measured using standard methods, such as flow cytometry.

The invention also provides a human regulatory ILC (ILCreg) which expresses detectable levels of one or more, such as 2 or 3, of the following cell surface markers: CD25 (IL2RA), CD127 and CTLA4. The human ILCreg preferably expresses detectable levels of CD25 (IL2RA), CD127, CTLA4, CD25 and CD127, CD25 and CTLA4, CD127 and CTLA4, or CD25, CD127 and CTLA4. Surface marker expression can be measured using standard methods, such as flow cytometry. The human ILCreg preferably further expresses a detectable level of FOXP3. The human ILCreg preferably further expresses detectable levels of one or more, such as 2, 3, 4, 5, 6, 7, 8, 9, 10, 11, 12, 13, 14, or 15, of the following genes: CCR5, FGGY, GATA3, GZMB, IL10, IL1R1, IL2RB, KAT2B, LGLS3, PIM1, PRDM1, RUNX1, SOX4, TNFRSF18 and TRAF1. The human ILCreg preferably expresses detectable levels of all of these genes. Gene expression is typically measured by measuring messenger RNA (mRNA) expression, for instance using RNA sequencing or single cell RNAseq (scRNAseq). It is worthy of note FOXP3, RUNX1, IL1R1 and CTLA4 are associated with human T-regulatory cells (Tregs). The human ILCreg preferably further secretes a detectable level of IL-10. This can be measured using a standard cytokine release assay.

In any of the embodiments above, the human ILCreg preferably does not express detectable levels of one or more, such as 2, 3, 4, 5 or 6, of CD3, CD4, CD19, CD20, TCR_{αβ}, and TCR^{γδ}. The human ILCreg preferably does not express detectable levels of one or more, such as 2, 3, 4 or 5, of CD3, CD4, CD19, CD20, and TCR_{αβ}. The human ILCreg preferably does not express detectable levels of one or more these markers at the cell surface and/or at the RNA level. The human ILCreg preferably does not express detectable levels of CD3 and CD4. The human ILCreg preferably does not express detectable levels of any of these markers. The human ILCreg preferably comprises a CD3⁻, CD4⁻, CD19⁻, CD20⁻, TCR_{αβ}⁻, TCR_{γδ}⁻ expression profile. The human ILCreg preferably comprises a CD3⁻, CD4⁻, CD19⁻, CD20⁻, TCR_{αβ}⁻ expression profile.

In some embodiments the ILCs comprise one or more of Group 1, Group 2 and Group 3 ILCs. In some embodiments, the ILCs comprise at least Group 1 ILCs. For example, the ILCs may comprise NK cells and/or ILC1 cells. NK cells may comprise a CD127-expression profile. The NK cells are preferably cytotoxic NK cells. ILC1 cells may comprise a CD127+expression profile. In some embodiments, the ILCs comprise or consist of NK cells. The NK cells are preferably cytotoxic NK cells. This is shown in Example 9.

In some embodiments, the ILCs comprise at least Group 2 ILCs. In some embodiments, the ILCs comprise at least Group 3 ILCs.

In some embodiments the ILCs comprise one or more of Group 1 ILCs, Group 2 ILCs, Group 3 ILCs and ILCregs. In some embodiments, the ILCs comprise at least Group 1 ILCs. In some embodiments, the ILCs comprise at least Group 2 ILCs. In some embodiments, the ILCs comprise at least Group 3 ILCs. In some embodiments, the ILCs comprise at least ILCregs. The ILCregs may be murine ILCregs as described in Wang et al. (2017). The ILCregs are preferably human ILCregs of the invention. The invention also provides a plurality or population of two or more human ILCregs of the invention. This is discussed in more detail below.

ILC1 cells may comprise the expression profile RORγt⁻ Klrg1⁻ NK1.1^{+/-} NKp46⁺ Eomes⁻ T-bet⁺. Human ILC1 cells may express detectable levels of one or more of the genes discussed above.

NK cells may comprise the expression profile T-bet⁺ Eomes⁺. Alternatively, NK cells may comprise the expression profile T-bet⁻ Eomes⁺. The NK cells are preferably cytotoxic NK cells. Human NK cells preferably express detectable levels of one or more of the genes discussed above.

The inventors have unexpectedly found that numerous different immune cell types can be expanded in the method of the present invention. In other words, numerous different immune cell types can unexpectedly be expanded under the same conditions. Thus, in some embodiments, the immune cells comprise a plurality of different immune cell subsets. Preferably, the immune cells comprise a plurality of different lymphoid cells, more preferably a plurality of different ILC groups. For example, the immune cells may comprise a plurality of Group 1 and Group 2 ILCs. In some embodiments the immune cells comprise a plurality of Group 1 and Group 3 ILCs. Alternatively, the immune cells may comprise a plurality of Group 2 and Group 3 ILCs. In some embodiments, the immune cells comprise a plurality of Group 1, Group 2 and Group 3 ILCs. In some embodiments, the immune cells comprise a plurality of Group 1 ILCs and ILCregs. In some embodiments, the immune cells comprise a plurality of ILCregs and Group 2 ILCs. In some embodiments, the immune cells comprise a plurality of ILCregs and Group 3 ILCs. In some embodiments, the immune cells comprise a plurality of Group 1 ILCs, Group 2 ILCs and ILCregs. In some embodiments, the immune cells comprise a plurality of Group 1 ILCs, Group 3 ILCs and ILCregs. In some embodiments, the immune cells comprise a plurality of Group 2 ILCs, Group 3 ILCs and ILCregs. In some embodiments, the immune cells comprise a plurality of Group 1 ILCs, Group 2 ILCs, Group 3 ILCs and ILCregs. The ILCregs may be murine ILCregs as described in Wang et al. (2017). The ILCregs are preferably human ILCregs of the invention.

Without wishing to be bound by theory, the present inventors believe that the ability to generate a plurality of different cell types more accurately represents the in vivo environment and so enables the generation of immune cells more suitable for cell therapy. In some embodiments, the method generates a heterogenous population of immune cells. The heterogenous population of immune cells may comprise or consist of the same immune cell type (e.g. ILCs or T-cells), but have varying expression profiles. For example, the heterogenous population of immune cells may comprise a heterogenous population of ILCs. The inventors believe that such a heterogenous population may have increased utility for in vitro research since it more accurately represents an in vivo population. Without wishing to be bound by theory, the inventors also believe that such a heterogenous population may have improved viability and function given its more accurate representation of an in vivo population. This may have particular utility in therapeutic applications.

Alternatively, in other embodiments the method generates a homologous population of immune cells.

In some embodiments, one or more of the different immune cell subsets in the plurality are enriched over time. For example, in embodiments comprising a plurality of different ILC groups, preferably the Group 1 ILCs, optionally ILC1 and/or NK cells, may be specifically enriched over time. In embodiments comprising a plurality of different ILC groups, the Group 3 ILCs may be enriched over time. Alternatively, in embodiments comprising a plurality of different ILC groups, the Group 2 ILCs may be enriched over time. In embodiments comprising a plurality of different ILC groups, the Group 1 and Group 3 ILCs may be enriched over time. In embodiments comprising a plurality of different ILC groups, the ILCregs may be enriched over time. The ILCregs may be murine ILCregs as described in Wang et al. (2017). The ILCregs are preferably human ILCregs of the invention.

In some embodiments, tissue-specific immune cells may be enriched over time. Tissue-specific immune cells are described herein. For example, when the epithelial organoid is an intestinal organoid, intestinal-specific immune cells may be enriched over time. When the epithelial organoid is a lung organoid, lung-specific immune cells may be enriched over time.

Advantageously, the present method can generate substantial numbers of immune cells from minimal starting material, for example from a negligible level of immune cells, such as only one immune cell. Thus, the method may also comprise a precursor step of expanding an immune cell to generate a plurality of the immune cell. Thus, the method may comprise:
ia) culturing an immune cell to generate immune cells; and
a) co-culturing the immune cells and at least one epithelial organoid, wherein the epithelial organoid comprises more epithelial cells than mesenchymal cells.

In some embodiments, the method is for the production and expansion of immune cells and wherein the method comprises before step (a):
co-culturing immune cell precursors and the epithelial organoid to expand the immune cell precursors and differentiate them into the immune cells.

As the skilled person will appreciate, immune cell precursors are progenitor cells of the immune cells. Thus, expansion and differentiation of the immune cell precursors leads to the generation of the immune cells. In some embodiments, the immune cell precursors are lymphoid precursors as defined above. In some embodiments, the method comprises a precursor step of expanding an immune cell precursor to generate a plurality of the immune cell precursor. Thus, in some embodiments the method is for the production and expansion of immune cells and wherein the method comprises before step (a):
culturing an immune cell precursor to generate immune cell precursors; and
co-culturing the immune cell precursors and the epithelial organoid to expand the immune cell precursors and differentiate them into the immune cells.

In some embodiments, the immune cell precursors are human immune cell precursors, equine immune cell precursors, feline immune cell precursors, canine immune cell precursors, bovine immune cell precursors, ovine immune cell precursors or murine immune cell precursors. In some embodiments, the immune cell precursors are human immune cell precursors or murine immune cell precursors. Optionally, the murine immune cell precursors are mouse immune cell precursors. In some embodiments, the immune cell precursors are human immune cell precursors.

The immune cell precursors may comprise or consist of murine or human ILC precursors.

Murine immune cell precursors may comprise or consist of murine ILC precursors. In such embodiments, it will be appreciated that the resulting immune cells are murine ILCs. In the context of the present invention, murine ILC precursors may comprise a CD127⁺, Lineage⁻ expression profile. In some embodiments, murine ILC precursors comprise a CD127⁺, Lineage⁻ alpha4beta7⁺ expression profile. In some embodiments, murine ILC precursors comprise a CD127⁺, Lineage⁻, PD-1⁺ expression profile. In some embodiments, murine ILC precursors comprises a CD127⁺, Lineage⁻, PD-1⁺, alpha4beta7⁺ expression profile. In some embodiments, murine ILC precursors comprise a CD127⁺, Lineage⁻, alpha4beta7⁺, PD-1⁺, Flt3⁻, CD25⁻, c-KIT⁺ expression profile. Various methods can be used to determine the expression profile, for example flow cytometry, fluorescence microscopy, RT-PCR and RNA-sequencing.

Human immune cell precursors may comprise or consist of human ILC precursors. In such embodiments, it will be appreciated that the resulting immune cells are human ILCs. In the context of the present invention, human ILC precursors may comprise a Lineage⁻, CD127⁺ expression profile. In some embodiments, human ILC precursors comprise a Lineage⁻, CD127⁺, CD7^{+/-}, c-Kit⁺, CRTh2⁻, KLRG1⁻, CD56⁻, NKp46⁻ expression profile. In some embodiments, human ILC precursors comprise a Lineage⁻, CD127⁺, c-Kit⁺ expression profile. Human ILC precursors may comprise a Lineage⁻, CD34⁺ expression profile. Alternatively, human ILC precursors may comprise a Lineage⁻, CD127⁺, CD45RA⁺, CD62L⁻ expression profile. Various methods can be used to determine the expression profile, for example flow cytometry, fluorescence microscopy, RT-PCR and RNA-sequencing.

“Lineage⁻”, as used herein in relation to mouse ILC precursors will be understood to refer to a CD3⁻ CD11b⁻ TER-119⁻ Ly-G6⁻ CD5⁻ CD19⁻ and NK1.1⁻ expression profile. “Lineage⁻”, as used herein in relation to human ILCs precursors will be understood to refer to a CD3⁻, CD4⁻, CD19⁻, CD20⁻, TCR_{αβ}⁻, TCR_{γδ}⁻ expression profile.

Preferably, the immune cell precursors are primary immune cell precursors. More preferably, the immune cell precursors are primary human immune cell precursors. By “primary cell” this will be understood to refer to a cell that has been obtained from a subject. Primary cells are not immortalised cells from a cell line.

The primary immune cell precursors may be autologous. Alternatively, the primary immune cell precursors may be allogeneic. In some embodiments, the primary immune cell precursors comprise a mixture of allogeneic and autologous immune cell precursors.

Alternatively, the immune cell precursors may comprise or consist of immortalised immune cell precursors from a cell line.

In some embodiments the immune cell precursors are primary immune cell precursors and the epithelial organoid is a primary epithelial organoid. In some embodiments, the immune cell precursors are primary immune cell precursors and the epithelial organoid is derived from stem cells, preferably iPSCs or adult stem cells. In some embodiments, the immune cell precursors are primary human immune cell precursors and the epithelial organoid is a primary human epithelial organoid. In some embodiments, the immune cell precursors are primary human immune cell precursors and the epithelial organoid is derived from human iPSCs or human adult stem cells.

In some embodiments the immune cell precursors are primary mouse immune cell precursors and the epithelial organoid is a primary human epithelial organoid. In some embodiments the immune cell precursors are primary mouse immune cell precursors and the epithelial organoid is derived from human iPSCs or human adult stem cells.

In some embodiments the immune cell precursors are primary human immune cell precursors and the epithelial organoid is a primary mouse epithelial organoid. Alternatively, the immune cell precursors may be primary human immune cell precursors and the epithelial organoid may be derived from mouse embryonic stem cells (ESC), mouse adult stem cells or mouse iPSCs, preferably derived from mouse ESCs or mouse adult stem cells.

In some embodiments the immune cell precursors are not mouse immune cell precursors and the epithelial organoid is not a mouse epithelial organoid.

In the context of the present invention, it will be appreciated that primary immune cells or primary immune cell precursors are isolated primary immune cells or primary immune cell precursors, given the in vitro nature of the method of the present invention. The primary immune cells or primary immune cell precursors may have been isolated from blood, bone marrow, foetal liver, tonsils or intestine. In some embodiments, the primary immune cells or immune cell precursors are isolated blood or bone marrow primary immune cells or primary immune cell precursors. In some embodiments the primary immune cells or immune cell precursors are isolated blood primary immune cells or primary immune cell precursors.

Advantageously, the present method can be maintained for prolonged periods of time, which enables the ongoing and reliable production of a large number of immune cells. In some embodiments, the immune cells and the epithelial organoid are co-cultured for at least about 72 hours. In some embodiments, the immune cells and the epithelial organoid are co-cultured for at least about 96 hours, at least about 120 hours, at least about 144 hours, at least about 168 hours, at least about 192 hours, at least about 216 hours, at least about 240 hours, at least about 264 hours, at least about 288 hours, at least about 312 hours, at least about 336 hours, at least about 504 hours, at least about 672 hours or at least about 1008 hours.

In some embodiments, the immune cells and the epithelial organoid are co-cultured for no more than about 1440 hours, about 1008 hours, about 720 hours, about 672 hours or about 504 hours.

In some embodiments, the immune cells and the epithelial organoid are co-cultured for of from about 96 hours to about 504 hours. Preferably, the immune cells and the epithelial organoid are co-cultured for of from about 120 hours to about 504 hours. More preferably, the immune cells and the epithelial organoid are co-cultured for of from about 168 hours to 336 hours.

During the method of the present invention, the immune cells preferably expand at a rate of at least 2-fold every about 24 hours. In some embodiments, the immune cells expand at a rate of at least 3-fold every about 24 hours.

The immune cells may expand at a rate of at least 4-fold every about 48 hours, optionally at a rate of at least about 6 fold every about 48 hours.

Optionally, the immune cells expand at a rate of at least 256-fold every about 168 hours.

In some embodiments, the immune cells expand at a rate of at least 200-fold, at least 250-fold, at least 300-fold, at least 350-fold, at least 400-fold, at least 450-fold, at least 500-fold, at least 550-fold, at least 600-fold, at least 650-fold, at least 700-fold, at least 750-fold, at least 800-fold, at least 850-fold or at least 900-fold about every 336 hours of co-culture.

In some embodiments, the immune cells expand at a rate of no more than 5000-fold, no more than 2000-fold, no more than 1000-fold, or no more than 950-fold about every 336 hours of co-culture. For example, the immune cells may expand at a rate of from 400-fold to 900-fold about every 336 hours of co-culture. In some embodiments, the immune cells expand at a rate of from 500-fold to 1000-fold about every 336 hours of co-culture. The immune cells may expand at a rate of from 500-fold to 800-fold about every 336 hours of co-culture. In some embodiments, the immune cells expand at a rate of about 750-fold about every 336 hours of co-culture.

The present invention also provides immune cells obtainable by the method of the present invention. Also provided is an in vitro population of innate lymphoid cells (ILCs), wherein the population comprises at least about 5×10³ ILCs.

The invention also provides an in vitro population of human ILCregs, wherein the population comprises at least about two human ILCregs of the invention. The population preferably comprises at least about 5, at least about 10, at least about 50, at least about 100, at least about 1000 or at least about 5×10³ human ILCregs of the invention. The human ILCregs of the invention may be any of those defined above.

In all embodiments of the invention, an in vitro population includes a population of cells in a format suitable for administration to a subject. This may include a vial, bag or needles containing the cells. The cells may be in a liquid solution or frozen form.

In some embodiments the population comprises at least about 5×10³ ILCs, at least about 1×10⁴ ILCs, at least about 5×10⁴ ILCs, at least about 1×10⁵ ILCs, at least about 5×10⁵ ILCs, at least about 1×10⁶ ILCs, at least about 5×10⁶ ILCs, at least about 1×10⁷ ILCs, at least about 5×10⁷ ILCs, at least about 1×10⁸ ILCs, at least about 5×10⁸ ILCs, at least about 1×10⁹ ILCs, at least about 5×10⁹ ILCs, at least about 1×10¹⁰ ILCs, at least about 5×10¹⁰ ILCs, at least about 1×10¹¹ ILCs, at least about 1×10¹² ILCs, at least about 1×10¹³ ILCs, at least about 1×10¹⁴ ILCs, at least about 1×10¹⁵ ILCs, at least about 1×10¹⁶ ILCs, at least about 1×10¹⁷ ILCs, at least about 1×10¹⁸ ILCs, at least about 1×10¹⁹ ILCs, at least about 1×10²⁰ ILCs, at least about 1×10²⁵ ILCs, at least about 1×10³⁰ ILCs, at least about 1×10³⁵ ILCs, at least about 1×10⁴⁰ ILCs, at least about 1×10⁴⁵ ILCs, at least about 1×10⁵⁰ ILCs, at least about 1×10⁶⁰ ILCs, at least about 1×10⁷⁰ ILCs, at least about 1×10⁸⁰ ILCs, at least about 1×10⁹⁰ ILCs, at least about 1×10¹⁰⁰ ILCs, at least about 1×10¹⁵⁰ ILCs, at least about 1×10²⁰⁰ ILCs, at least about 1×10²⁵⁰ ILCs, at least about 1×10³⁰⁰ ILCs, at least about 1×10³⁵⁰ ILCs, at least about 1×10⁴⁰⁰ ILCs, at least about 1×10⁴⁵⁰ ILCs, at least about 1×10⁵⁰⁰ ILCs, at least about 1×10⁵⁵⁰ ILCs, at least about 1×10⁶⁰⁰ ILCs, at least about 1×10⁶⁵⁰ ILCs, at least about 1×10⁷⁰⁰ ILCs, at least about 1×10⁷⁵⁰ ILCs, at least about 1×10⁸⁵⁰ ILCs, at least about 1×10⁹⁵⁰ ILCs, at least about 1×10¹⁰⁰⁰ ILCs, at least about 1×10²⁰⁰⁰ ILCs, at least about 1×10³⁰⁰⁰ ILCs, at least about 1×10⁴⁰⁰⁰ ILCs, at least about 1×10⁴⁰⁰⁰ ILCs, at least about 1×10⁵⁰⁰⁰ ILCs, at least about 1×10⁶⁰⁰⁰ ILCs, at least about 1×10⁷⁰⁰⁰ ILCs, at least about 1×10⁸⁰⁰⁰ ILCs, at least about 1×10⁹⁰⁰⁰ ILCs or at least about 1×10¹⁰⁰⁰⁰ ILCs. In any of these embodiments, the ILCs may be ILCregs, murine ILCregs or human ILCregs of the invention.

In some embodiments the population comprises at least about 1×10²⁰ ILCs. In some embodiments the population comprises at least about 1×10¹⁰⁰ ILCs. In some embodiments the population comprises at least about 1×10⁵⁰⁰ ILCs. In some embodiments the population comprises at least about 1×10¹⁰⁰⁰ ILCs. In any of these embodiments, the ILCs may be ILCregs, murine ILCregs or human ILCregs of the invention.

In some embodiments the population comprises no more than about 1×10¹⁰⁰⁰⁰ ILCs. In some embodiments the population comprises no more than about 1×10⁵⁰⁰ ILCs. In some embodiments the population comprises of from about 5×10⁴ ILCs to about 1×10¹⁰⁰⁰⁰ ILCs. In some embodiments the population comprises of from about 1×10²⁰⁰ ILCs to about 1×10¹⁰⁰⁰⁰ ILCs. In any of these embodiments, the ILCs may be ILCregs, murine ILCregs or human ILCregs of the invention.

The ILCs may be as defined above. For example, the in vitro population of ILCs may comprise or consist of human or murine ILCs. The in vitro population of ILCs may comprise one or more of Group 1, Group 2 and Group 3 ILCs. The in vitro population of ILCs may comprise one or more of Group 1 ILCs, Group 2 ILC, Group 3 ILCs and ILCregs. Murine and human Group 1, Group 2 and Group 3 ILCs can be identified as discussed above. Mouse ILCregs can be identified as described in Wang et al. (2017). Human ILCregs can be identified as discussed above.

In some embodiments, the ILCs comprise at least Group 1 ILCs. The Group 1 ILCs may comprise NK cells and/or ILC1 cells. The NK cells are preferably cytotoxic NK cells. This is shown in Example 9.

The in vitro population of ILCs may comprise a plurality of Group 1 and Group 2 ILCs. In some embodiments the in vitro population of ILCs comprise a plurality of Group 1 and Group 3 ILCs. Alternatively, the in vitro population of ILCs may comprise a plurality of Group 2 and Group 3 ILCs. In some embodiments, the in vitro population of ILCs comprise a plurality of Group 1, Group 2 and Group 3 ILCs. In some embodiments, the in vitro population of ILCs comprises a plurality of Group 1 ILCs and ILCregs. In some embodiments, the in vitro population of ILCs comprises a plurality of ILCregs and Group 2 ILCs. In some embodiments, the in vitro population of ILCs comprises a plurality of ILCregs and Group 3 ILCs. In some embodiments, the in vitro population of ILCs comprises a plurality of Group 1 ILCs, Group 2 ILCs and ILCregs. In some embodiments, the in vitro population of ILCs comprises a plurality of Group 1 ILCs, Group 3 ILCs and ILCregs. In some embodiments, the in vitro population of ILCs comprises a plurality of Group 2 ILCs, Group 3 ILCs and ILCregs. In some embodiments, the in vitro population of ILCs comprises a plurality of Group 1 ILCs, Group 2 ILCs, Group 3 ILCs and ILCregs. The ILCregs may be murine ILCregs as described in Wang et al. (2017). The ILCregs are preferably the human ILCregs of the invention.

The in vitro population of ILCs may comprise at least about 1%, at least about 2%, at least about 5%, at least about 10%, at least about 20%, at least about 25%, at least about 30%, at least about 40%, at least about 50%, at least about 60%, at least about 70%, at least about 80% or at least about 90% Group 1 ILCs.

The in vitro population of ILCs may comprise at least about 1%, at least about 2%, at least about 5%, at least about 10%, at least about 20%, at least about 25%, at least about 30%, at least about 40%, at least about 50%, at least about 60%, at least about 70%, at least about 80% or at least about 90% NK cells. The NK cells are preferably cytotoxic NK cells.

The in vitro population of ILCs may comprise at least about 1%, at least about 5%, at least about 7% or at least about 10% Group 2 ILCs. In some embodiments, the in vitro population of ILCs comprises no more than about 20% or no more than about 10% Group 2 ILCs.

The in vitro population of ILCs may comprise at least about 1%, at least about 2%, at least about 5%, at least about 10%, at least about 20%, at least about 25%, at least about 30%, at least about 40%, at least about 50%, at least about 60% or at least about 70% Group 3 ILCs. In some embodiments, the in vitro population of ILCs comprises no more than about 90%, no more than about 80%, no more than about 75% or no more than about 70% Group 3 ILCs.

The in vitro population of ILCs may comprise at least about 0.1%, at least about 0.2%, at least about 0.5%, at least about 0.8%, at least about 1%, at least about 2%, at least about 5%, at least about 10%, at least about 20%, at least about 25%, at least about 30%, at least about 40%, at least about 50%, at least about 60%, at least about 70%, at least about 80% or at least about 90% ILCregs, murine ILCregs or human ILCregs of the invention. The in vitro population of human ILCregs of the invention may comprise at least about 0.1%, at least about 0.2%, at least about 0.5%, at least about 0.8%, at least about 1%, at least about 2%, at least about 5%, at least about 10%, at least about 20%, at least about 25%, at least about 30%, at least about 40%, at least about 50%, at least about 60%, at least about 70%, at least about 80% or at least about 90% human ILCregs of the invention.

Without wishing to be bound by theory, the present inventors believe that ILC ratios can become dysregulated in some diseases. Particular tissue-specific ratios may therefore be especially useful in the treatment of such diseases.

In some embodiments the population comprises about 95% NK cells. This frequency of NK cells may be associated with healthy human lung tissue, and so such a frequency may be especially beneficial for the treatment of lung diseases. The NK cells are preferably cytotoxic NK cells. This is shown in Example 9.

In some embodiments the population comprises about 60% Group 1 ILCs and about 20-40% Group 3 ILCs. In some embodiments the population comprises about 60% Group 1 ILCs, about 35% Group 3 ILCs and about 5% Group 2 ILCs.

In some embodiments the population comprises about 90% Group 3 ILCs and about 10% Group 1 ILCs. In some embodiments the population comprises an undetectable percentage of Group 2 ILCs. Such frequencies may be associated with healthy human colon tissue, and so such frequencies may be especially beneficial for the treatment of colon or intestinal diseases.

In some embodiments the population comprises of from about 60 to 70% Group 3 ILCs and about of from about 20 to 30% NK cells. For example, the population may comprise about 66% Group 3 ILCs and about 27% NK cells. Such populations may be associated with healthy human adenoid tissue. The NK cells are preferably cytotoxic NK cells. This is shown in Example 9.

In some embodiments the population comprises of from about 40% to 50% Group 3 ILCs and of from about 30 to 50% Group I ILCs. For example, the population may comprise about 48% Group 3 ILCs and about 40% Group I ILCs. Such populations may be associated with healthy human tonsil tissue.

In some embodiments, the in vitro population of ILCs is an in vitro population of NK cells. The NK cells are preferably cytotoxic NK cells. This is shown in Example 9.

In some embodiments, the in vitro population of ILCs is a heterogenous population of ILCs. In other embodiments the in vitro population of ILCs is a homologous population of ILCs.

In some embodiments, at least 10% of the ILCs express detectable levels of one or more of NKp44, NKp46, CD56, c-KIT, ST2, CRTh2, Klrg1, CD122, CD127, T-bet, GATA3, ROR-yt, ID2, IL-10, IL-5, IL-13, IL-17a, IFN-γ, Amphiregulin, Granzyme B, Perforin, or TGFβ. In some embodiments the ILCs express detectable levels of one or more of IL-22, IL-5, IL-13, IL-17a IFN-γ, Amphiregulin, Granzyme B, Perforin, NKp44, T-bet, GATA3, ROR-yt and HLA-DR. The ILCs are preferably capable of antigen processing. This is shown in Example 7.

In some embodiments, at least 10% of the ILCs express detectable levels of HLA-DR. In some embodiments, the ILCs express detectable levels of HLA-DR. The ILCs are preferably capable of antigen processing. This is shown in Example 7.

In some embodiments, at least 10% of the ILCs express detectable levels of receptors of one or more of IL-23, IL-1B, IL-15, IL-12, IL-18, IL-25 and IL-33.

In some embodiments, the in vitro population of ILCs comprise Group 1 ILCs. The Group 1 ILCs may express a detectable level of IFN-γ. In some embodiments, at least 10%, at least 20%, at least 30%, at least 40%, at least 50%, at least 60%, at least 70%, at least 80% or at least 90% of the Group 1 ILCs express a detectable level of IFN-γ. In some embodiments at least 30% of the Group 1 ILCs express a detectable level of IFN-γ.

The ILCs may express a detectable level of T-bet. In some embodiments at least 10%, at least 20%, at least 30%, at least 40%, at least 50%, at least 60%, at least 70%, at least 80% or at least 90% of the ILCs express a detectable level of T-bet.

In some embodiments, at least 20%, at least 30%, at least 40%, at least 50%, at least 60%, at least 70%, at least 80% or at least 90% of the ILCs express a detectable level of GATA3. In some embodiments at least 30% of the ILCs express a detectable level of GATA3. In some embodiments at least 50% of the ILCs express a detectable level of GATA3. The ILCs may express a detectable level of GATA3.

At least 20%, at least 30%, at least 40%, at least 50%, at least 60%, at least 70%, at least 80% or at least 90% of the ILCs may express a detectable level of IL-13. In some embodiments at least 50%, preferably at least 60% of the ILCs express a detectable level of IL-13. In some embodiments at least 20%, at least 30%, at least 40%, at least 50%, at least 60%, at least 70%, at least 80% or at least 90% of the ILCs may express a detectable level of IL-5. In some embodiments at least 40% of the ILCs express a detectable level of IL-5. In some embodiments at least 20%, at least 30%, at least 40%, at least 50%, at least 60%, at least 70%, at least 80% or at least 90% of the ILCs co-express a detectable level of IL-5 and IL-13. In some embodiments at least 50% of the ILCs co-express a detectable level of IL-5 and IL-13.

The ILCs may express a detectable level of IL-5 and/or IL-13. In some embodiments the ILCs co-express a detectable level of IL-5 and IL-13.

At least 10%, at least 20%, at least 30%, at least 40%, at least 50%, at least 60%, at least 70%, at least 80% or at least 90% of the ILCs may express a detectable level of IL-22. In some embodiments at least 30%, preferably at least 40% of the ILCs express a detectable level of IL-22. The ILCs may express a detectable level of IL-22.

At least 1%, at least 2%, at least 5%, at least 10%, at least 20%, at least 30%, at least 40%, at least 50%, at least 60%, at least 70%, at least 80% or at least 90% of the ILCs may express a detectable level of NKp44. In some embodiments at least 10% of the ILCs express a detectable level of NKp44. In some embodiments at least 15% of the ILCs express a detectable level of NKp44. In some embodiments at least 20% of the ILCs express a detectable level of NKp44. In some embodiments of from about 10% to about 30%, preferably of from about 15% to about 25% of the ILCs express a detectable level of NKp44. The ILCs may express a detectable level of NKp44.

In some embodiments at least 10%, at least 20%, at least 30%, at least 40%, at least 50%, at least 60%, at least 70%, at least 80% or at least 90% of the ILCs express IL-22. In some embodiments at least 20% of the ILCs express IL-22. In some embodiments at least 50% of the ILCs express IL-22. In some embodiments of from about 10% to about 70% of the ILCs express IL-22.

In some embodiments at least 10%, at least 20%, at least 30%, at least 40%, at least 50%, at least 60%, at least 70%, at least 80% or at least 90% of the ILCs express IL-17a. In some embodiments at least 20% of the ILCs express IL-17a. In some embodiments at least 50% of the ILCs express IL-17a. In some embodiments of from about 10% to about 70% of the ILCs express IL-17a.

Preferably, at least 2% of the in vitro population of ILCs express a detectable level of NKp44. In some embodiments, at least 5% of the in vitro population of ILCs express a detectable level of NKp44. In some embodiments, at least 10% of the in vitro population of ILCs express a detectable level of NKp44. In some embodiments, at least 15% of the in vitro population of ILCs express a detectable level of NKp44. In some embodiments, at least 20% of the in vitro population of ILCs express a detectable level of NKp44. In some embodiments, at least 50% of the in vitro population of ILCs express a detectable level of NKp44. In some embodiments, the in vitro population of ILCs express a detectable level of NKp44.

In some embodiments, the in vitro population of ILCs comprise Group 2 ILCs. In some embodiments, at least 20%, at least 30%, at least 40%, at least 50%, at least 60%, at least 70%, at least 80% or at least 90% of the Group 2 ILCs express a detectable level of GATA3. In some embodiments at least 30% of the Group 2 ILCs express a detectable level of GATA3. In some embodiments at least 50% of the Group 2 ILCs express a detectable level of GATA3. The Group 2 ILCs may express a detectable level of GATA3.

At least 20%, at least 30%, at least 40%, at least 50%, at least 60%, at least 70%, at least 80% or at least 90% of the Group 2 ILCs may express a detectable level of IL-13. In some embodiments at least 50%, preferably at least 60% of the Group 2 ILCs express a detectable level of IL-13. In some embodiments at least 20%, at least 30%, at least 40%, at least 50%, at least 60%, at least 70%, at least 80% or at least 90% of the Group 2 ILCs may express a detectable level of IL-5. In some embodiments at least 40% of the Group 2 ILCs express a detectable level of IL-5. In some embodiments at least 20%, at least 30%, at least 40%, at least 50%, at least 60%, at least 70%, at least 80% or at least 90% of the Group 2 ILCs co-express a detectable level of IL-5 and IL-13. In some embodiments at least 50% of the Group 2 ILCs co-express a detectable level of IL-5 and IL-13.

The Group 2 ILCs may express a detectable level of IL-5 and/or IL-13. In some embodiments the Group 2 ILCs co-express a detectable level of IL-5 and IL-13.

At least 10%, at least 20%, at least 30%, at least 40%, at least 50%, at least 60%, at least 70%, at least 80% or at least 90% of the Group 2 ILCs may express a detectable level of IL-22. In some embodiments at least 30%, preferably at least 40% of the Group 2 ILCs express a detectable level of IL-22. The Group 2 ILCs may express a detectable level of IL-22.

The in vitro population of ILCs may comprise Group 3 ILCs. At least 2%, at least 5%, at least 10%, at least 20%, at least 30%, at least 40%, at least 50%, at least 60%, at least 70%, at least 80% or at least 90% of the Group 3 ILCs may express a detectable level of NKp44. In some embodiments at least 10% of the Group 3 ILCs express a detectable level of NKp44. In some embodiments at least 15% of the Group 3 ILCs express a detectable level of NKp44. In some embodiments at least 20% of the Group 3 ILCs express a detectable level of NKp44. In some embodiments of from about 10% to about 30%, preferably of from about 15% to about 25% of the Group 3 ILCs express a detectable level of NKp44. The Group 3 ILCs may express a detectable level of NKp44.

In some embodiments at least 10%, at least 20%, at least 30%, at least 40%, at least 50%, at least 60%, at least 70%, at least 80% or at least 90% of the Group 3 ILCs express IL-22. In some embodiments at least 20% of the Group 3 ILCs express IL-22. In some embodiments at least 50% of the Group 3 ILCs express IL-22. In some embodiments of from about 10% to about 70% of the Group 3 ILCs express IL-22.

In some embodiments at least 10%, at least 20%, at least 30%, at least 40%, at least 50%, at least 60%, at least 70%, at least 80% or at least 90% of the Group 3 ILCs express IL-17a. In some embodiments at least 20% of the Group 3 ILCs express IL-17a. In some embodiments at least 50% of the Group 3 ILCs express IL-17a. In some embodiments of from about 10% to about 70% of the Group 3 ILCs express IL-17a.

In some embodiments at least about 10%, at least about 20%, at least about 30%, at least about 40%, at least about 50%, at least about 60%, at least about 70%, at least about 80% or at least about 90% of the ILCregs express or secrete a detectable level of IL-10. The ILCregs may be murine ILCregs or human ILCregs of the invention.

In some embodiments at least about 10%, at least about 20%, at least about 30%, at least about 40%, at least about 50%, at least about 60%, at least about 70%, at least about 80% or at least about 90% of the ILCregs express a detectable level of FOXP3. The ILCregs may be murine ILCregs or human ILCregs of the invention.

In some embodiments at least about 10%, at least about 20%, at least about 30%, at least about 40%, at least about 50%, at least about 60%, at least about 70%, at least about 80% or at least about 90% of the ILCregs express a detectable level of CD25 (IL2RA). The ILCregs may be murine ILCregs or human ILCregs of the invention.

In some embodiments at least about 10%, at least about 20%, at least about 30%, at least about 40%, at least about 50%, at least about 60%, at least about 70%, at least about 80% or at least about 90% of the ILCregs express a detectable level of CD127. The ILCregs may be murine ILCregs or human ILCregs of the invention.

In some embodiments at least about 10%, at least about 20%, at least about 30%, at least about 40%, at least about 50%, at least about 60%, at least about 70%, at least about 80% or at least about 90% of the ILCregs express a detectable level of CTLA4. The ILCregs may be murine ILCregs or human ILCregs of the invention. At least 50% of the in vitro population of ILCs may co-express detectable levels of two or more cytokines. The ILCregs may be murine ILCregs as described in Wang et al. (2017). The ILCs may be human ILCregs of the invention.

The in vitro population of ILCs may be a stimulated in vitro population of ILCs. Various stimulation methods are known to those skilled in the art. For example, the population may comprise a PMA and Ionomycin stimulated in vitro population of ILCs. Preferably, a stimulated in vitro population of ILCs expresses detectable levels of one or more cytokines as described above. In any of these embodiments, the ILCs may be ILCregs, murine ILCregs or human ILCregs of the invention.

The in vitro population of ILCs may have a tissue-specific imprint. By “tissue-specific imprint”, this will be understood to refer to a genetic signature and phenotype specific of in vivo immune cells of a particular organ. Thus, in some embodiments the in vitro population of ILCs comprises or consists of a population of tissue-specific ILCs. Such tissue-specific ILCs may have utility for the treatment of particular diseases and/or may have improved homing capacity to the specific tissue when administered to a subject. In some embodiments, the in vitro population of ILCs comprise or consist of skin, intestinal, lung, thymic, thyroid, reproductive, bladder, kidney, pancreas, oral mucosal or liver specific ILCs. In some embodiments, the in vitro population of ILCs comprises or consists of skin, intestinal, lung, thyroid, reproductive, bladder, kidney, pancreas, oral mucosal or liver specific ILCs. In some embodiments, the in vitro population of ILCs comprises or consists of intestinal, lung, reproductive or oral mucosal specific ILCs. In any of these embodiments, the ILCs may be ILCregs, murine ILCregs or human ILCregs of the invention.

Reproductive-specific ILCs may comprise fallopian tube specific ILCs, ovary specific ILCs, prostate specific ILCs, endometrium specific ILCs, cervix specific ILCs, vaginal specific ILCs and testes-specific ILCs. Reproductive-specific ILCs may be selected from fallopian tube specific ILCs, ovary specific ILCs, prostate specific ILCs and endometrium specific ILCs. In any of these embodiments, the ILCs may be ILCregs, murine ILCregs or human ILCregs of the invention.

Oral mucosal specific ILCs may comprise salivary gland taste bud specific ILCS, lingual region specific ILCs and oesophagus specific ILCs. In some embodiments, the oral mucosal specific ILCs are oesophageal specific ILCs. In any of these embodiments, the ILCs may be ILCregs, murine ILCregs or ILCregs of the invention.

In some embodiments, the in vitro population of ILCs comprises or consists of intestinal-specific ILCs and/or lung-specific ILCs. The intestinal specific ILCs may be small intestinal-specific ILCs and/or lung specific ILCs. In any of these embodiments, the ILCs may be ILCregs, murine ILCregs or ILCregs of the invention.

Alternatively, the in vitro population of ILCs may comprise or consist of epithelial cancer specific ILCs. In some embodiments the in vitro population of ILCs may comprise or consist of skin cancer specific ILCs, intestinal cancer specific ILCs, lung cancer specific ILCs, thymic cancer specific ILCs, thyroid cancer specific ILCs, reproductive cancer specific ILCs, bladder cancer specific ILCs, kidney cancer specific ILCs, pancreas cancer specific ILCs, oral mucosal cancer specific ILCs or liver cancer specific ILCs the in vitro population of ILCs may comprise or consist of skin cancer specific ILCs, intestinal cancer specific ILCs, lung cancer specific ILCs, thyroid cancer specific ILCs, reproductive cancer specific ILCs, bladder cancer specific ILCs, kidney cancer specific ILCs, pancreas cancer specific ILCs, oral mucosal cancer specific ILCs or liver cancer specific ILCs. In any of these embodiments, the ILCs may be ILCregs, murine ILCregs or ILCregs of the invention.

In some embodiments, the ILCs are human ILCs, equine ILCs, feline ILCs, canine ILCs, bovine ILCs, ovine ILCs or murine ILCs. In some embodiments, the ILCs are human ILCs or murine ILCs. Optionally, the murine ILCs are mouse ILCs. In some embodiments, the ILCs are human ILCs. The human ILCs may be ILCregs, murine ILCregs or human ILCregs of the invention.

The in vitro population of ILCs may comprise or consist of small intestinal specific mouse ILC1 cells. Small intestinal specific mouse ILC1 cells may comprise a Eomes⁻, CD127⁺, CD49a^{+/-}, CD49b⁻, TRAIL^{+/-}, CD103⁻, CD69⁺, CD200r1⁺, CXCR6^{+/-}, CD61⁺ expression profile.

The in vitro population of ILCs may comprise or consist of liver specific mouse ILC1 cells. Liver specific mouse ILC1 cells may comprise a Eomes⁻, CD127^{+/-}, CD49a⁺, CD49b⁻, TRAIL⁺, CD103⁻, CD69⁺, CD200r1⁺, CXCR6⁺, CD61⁺ expression profile.

The in vitro population of ILCs may comprise or consist of oral mucosal specific mouse ILC1 cells. Oral mucosal mouse ILC1 cells may comprise a Eomes⁺, CD127⁻, CD49a⁺, CD49b⁺, TRAIL⁺, CD103^{+/-}, CD69⁺, CD200r1⁺, CXCR6⁺, CD61⁺ expression profile.

The in vitro population of ILCs may comprise or consist of small intestinal specific human ILC1 cells. Small intestinal specific human ILC1 cells may comprise a T-bet⁺, Eomes^{+/-}, CD56^{+/-}, CD127⁺, CD49a⁻, CD103⁻, CD69^{+/-}, NKp44^{+/-}, CD16⁻ expression profile. The small intestinal specific human ILC1 cells may express detectable levels of one or more specific genes as described above.

The in vitro population of ILCs may comprise or consist of lung specific human ILC1 cells. Lung specific human ILC1 cells may comprise a T-bet^{+/-}, Eomes^{+/-}, CD56^{+/-}, CD127⁺, CD49a⁻, CD103⁻, CD69^{+/-}, NKp44^{+/-}, CD16⁻ expression profile. The lung specific human ILC1 cells may express detectable levels of one or more specific genes as described above.

The in vitro population of ILCs may comprise or consist of liver specific human ICL1 cells. Liver specific human ILC1 cells may comprise a T-bet⁺, Eomes, CD56^{+/-}, CD127⁻, CD49a⁺, CD69⁺, CD16⁻ expression profile. The liver specific human ILC1 cells may express detectable levels of one or more specific genes as described above.

The in vitro population of ILCs may comprise or consist of liver specific mouse ILC2 cells. Liver specific mouse ILC2 cells may comprise a ST2⁺, IL17RB⁺, KLRG1⁺, CD69⁺ expression profile.

The in vitro population of ILCs may comprise or consist of small intestine specific mouse ILC2 cells. Small intestine specific mouse ILC2 cells may comprise a ST2^{+/-}, IL17RB⁺, IL18R1⁻, KLRG1⁺, CD69⁺, NMUR1⁺, VPAC1/2⁺, CCR4⁺, CCR8^{+/-}, MHCII^{+/-} expression profile.

The in vitro population of ILCs may comprise or consist of lung specific mouse ILC2 cells. Lung specific mouse ILC2 cells may comprise a ST2⁺, IL17RB⁺, IL18R1^{+/-}, KLRG1^{+/-}, CD69⁺, NMUR1⁺, VPAC1/2⁺, CCR4⁺, CCR8⁺ expression profile.

The in vitro population of ILCs may comprise or consist of lung specific human ILC2 cells. Lung specific human ILC2 cells may comprise a CRTH2^{+/-}, KLRG1^{+/-}, CD117^{+/-}, CD49a^{+/-}, ICOS^{+/-}, CD69^{+/-}, NKp30⁻, CD25^{+/-}, CCR6^{+/-}, CCR4^{+/-} expression profile. The lung specific human ILC2 cells may express detectable levels of one or more specific genes as described above.

The in vitro population of ILCs may comprise or consist of small intestine specific human ILC2 cells. Small intestine specific human ILC2 cells may comprise a CRTH2^{+/-}, KLRG1^{+/-}, CD117^{+/-}, CD49a^{+/-}, ICOS^{+/-}, CD69^{+/-}, NKp30^{+/-}, CD25^{+/-}, CCR6^{+/-}, CCR4^{+/-}, HLA-DR^{+/-} expression profile. The small intestinal specific human ILC2 cells may express detectable levels of one or more specific genes as described above.

The in vitro population of ILCs may comprise or consist of liver specific human ILC2 cells. Liver specific human ILC2 cells may comprise a CRTH2^{+/-}, KLRG1^{+/-}, CD117^{+/-}, CD49a^{+/-}, ICOS^{+/-}, CD69^{+/-}, NKp30^{+/-}, CD25^{+/-}, CCR6^{+/-}, CCR4^{+/-} expression profile. The liver specific human ILC2 cells may express detectable levels of one or more specific genes as described above.

The in vitro population of ILCs may comprise or consist of small intestine specific mouse ILC3 cells. Small intestine specific mouse ILC3 cells may comprise a CXCR6^{+/-}, CCR7⁺, CCR9⁺, α4β7⁺, MHCII⁺ expression profile.

The in vitro population of ILCs may comprise or consist of small intestine specific human ILC3 cells. Small intestine specific human ILC3 cells may comprise a NKp44^{+/-}, CD69^{+/-}, CCR7⁻, ICOS^{+/-}, CD39^{+/-}, CD45RA^{+/-}, NRP1^{+/-}, CD25^{+/-}, HLA-DR^{+/-}, IL1R1; IL23R⁺ expression profile. The small intestinal specific human ILC3 cells may express detectable levels of one or more specific genes as described above.

The in vitro population of ILCs may comprise or consist of lung specific human ILC3 cells. Lung specific human ILC3 cells may comprise a NKp44^{+/-}, CD69^{+/-}, CCR7⁻, ICOS^{+/-}, CD39^{+/-}, CD45RA^{+/-}, NRP1^{+/-}, CD25⁺, HLA-DR-expression profile. The lung specific human ILC3 cells may express detectable levels of one or more specific genes as described above.

Preferably, the ILCs are primary ILCs. More preferably, the ILCs are primary human ILCs. The primary ILCs may be autologous. Alternatively, the primary ILCs may be allogeneic. In some embodiments, the population comprises a mixture of allogeneic and autologous ILCs.

Preferably, the ILCregs are primary ILCregs. The primary ILCregs may be autologous. Alternatively, the primary ILCregs may be allogeneic. In some embodiments, the population comprises a mixture of allogeneic and autologous ILCregs.

Preferably, the human ILCregs are primary human ILCregs. The primary human ILCregs may be autologous. Alternatively, the primary human ILCregs may be allogeneic. In some embodiments, the population comprises a mixture of allogeneic and autologous ILCregs. The human ILCregs are preferably human ILCregs of the invention. Alternatively, the ILCs may comprise or consist of immortalised immune cells from a cell line. Alternatively, the ILCregs may comprise or consist of immortalised ILCregs or an ILCreg cell line.

In some embodiments, at least about 10% of the in vitro population comprises an exogenous polynucleotide. By “exogenous polynucleotide”, this will be understood to refer to a polynucleotide which has been introduced into the ILC or a precursor of the ILC, such that the ILC or a precursor of the ILC is genetically modified. Thus, in some embodiments the in vitro population of ILCS is a genetically modified in vitro population of ILCs. Typically, the exogenous polynucleotide is recombinant. The exogenous polynucleotide typically encodes an exogenous polypeptide. It will be appreciated that the exogenous polypeptide may be a polypeptide which is endogenous to the ILC but is expressed in the cell at higher levels following introduction of the exogenous polynucleotide by genetic modification Alternatively, the exogenous polypeptide may be a polypeptide which is not naturally expressed in the ILC. In any of these exogenous polynucleotide and polypeptide embodiments including those discussed below, the ILC or ILCs may be an ILCreg/ILCregs, a murine ILCreg/murine ILCregs or a human ILCreg of the invention/human ILCregs of the invention.

In some embodiments at least about 20%, at least about 30%, at least about 40%, at least about 50%, at least about 60%, at least about 70%, at least about 80% or at least about 90% of the in vitro population comprise an exogenous polynucleotide.

In some embodiments, at least about 10% of the in vitro population express the exogenous polypeptide encoded by the exogenous polynucleotide at a detectable level. In some embodiments at least about 20%, at least about 30%, at least about 40%, at least about 50%, at least about 60%, at least about 70%, at least about 80% or at least about 90% of the in vitro population express the exogenous polypeptide encoded by the exogenous polynucleotide at a detectable level.

The exogenous polypeptide may comprise a marker protein or an immunotherapeutic molecule. The marker protein may otherwise be referred to as a reporter protein. Suitable marker proteins include, but are not necessarily limited to GFP, a MYC epitope tag or a FLAG epitope tag.

In some embodiments, the exogenous polypeptide further comprises a purification tag. As the skilled person will appreciate, a purification tag can assist with purification. Examples of purification tags include but are not necessarily limited to a His-tag, Arg-tag, T7-tag, Strep-tag, S-tag, aptamer-tag, V5 tag, or AviTag™. Various other tags are well known in the art.

The immunotherapeutic molecule may be any immunotherapeutic molecule which may further increase the immunotherapeutic use of the in vitro population of ILCs. For example, the immunotherapeutic molecule may comprise an enzyme, an antibody, an antigen, a chimeric antigen receptor (CAR), MHC class II cell receptor, chemokine receptor and/or a cytokine. The MHC class II cell receptor may preferably comprise HLA-DR.

In some embodiments the immunotherapeutic molecule comprises or consists of a chimeric antigen receptor (CAR). Chimeric antigen receptors are immune cell receptors which have been genetically engineered to confer the ability to target a specific antigen or antigens.

Generally, chimeric antigen receptors are specific for one or more cancer-associated antigens. As such, chimeric antigen receptors are commonly used in the treatment of cancer.

Various CARs are known to those skilled in the art. In particular, the CAR may comprise or consist of a first, second, third, or fourth generation CAR.

First-generation CARs comprise or consist of a binding domain that is capable of specifically binding to an epitope on a target antigen, a transmembrane domain, and one or more intracellular signalling domains. The extracellular binding domain may comprise a single-chain variable fragment (scFv) from a monoclonal antibody. A first-generation CAR typically comprises a CD34 chain domain or a variant thereof as the intracellular signalling domain, which is the primary transmitter of signals.

In addition to the components specified for first-generation CARs, second-generation CARS also contain a co-stimulatory domain, such as CD28 and/or 4-1BB. The inclusion of an intracellular co-stimulatory domain improves T-cell proliferation, cytokine secretion, resistance to apoptosis, and in vivo persistence. The co-stimulatory domain of a second-generation CAR is typically in cis with and upstream of the one or more intracellular signalling domains.

Third-generation CARs combine multiple co-stimulatory domains in cis with one or more intracellular signalling domains, to augment T-cell activity. For example, a third-generation CAR may comprise co-stimulatory domains derived from CD28 and 41BB, together with an intracellular signalling domain derived from CD3 zeta. Other third-generation CARs may comprise co-stimulatory domains derived from CD28 and OX40, together with an intracellular signalling domain derived from CD3 zeta.

Fourth-generation CARs (also known as TRUCKs or armoured CARs), combine the features of a second-generation CAR with further factors to enhance anti-tumour activity (e.g., cytokines, co-stimulatory ligands, chemokines receptors or further chimeric receptors of immune regulatory or cytokine receptors). The factors may be in trans or in cis with the CAR, typically in trans with the CAR.

In some embodiments, the CAR is specific for a cancer antigen. The cancer antigen may be a solid tumour cancer antigen. By “specific”, in the context of the CAR, this will be understood to refer to being capable of specifically binding to a target antigen.

Cancer antigens include, but are not necessarily limited to Erbb1, Erbb3, Erbb4, Erbb2, mucins, PSMA, carcinoembryonic antigen (CEA), mesothelin, GD2, MUC1, folate receptor, NKG2D ligands, ligands bound by other NK receptors such as NKp30, NKp44 or NKp46, GPC3, CAIX, FAP, NY-ESO-1, gp100, PSCA, ROR1, PD-L1, PD-L2, EpCAM, EGFRvIII, CD19, CD20, CD22, GD3, CLL-1, ductal epithelial mucin, CA-125, GP36, TAG-72, glycosphingolipids, glioma-associated antigen, beta-hCG, AFP (alpha-fetoprotein) and lectin-reactive AFP, thyroglobulin, receptor for advanced glycation end products (RAGE), TERT, telomerase, carboxylesterase, M-CSF, M-CSF receptor, PSA, tyrosinase, survivin, PCTA-1, melanoma-associated antigen (MAGE), for example MAGE A1, MAGE A2, MAGE A4, MAGE A8, CD22, IGF-1, IGF-2, IGF-1 receptor, MHC-associated tumour peptide, 5T4, tumour stroma-associated antigens, WT1, MLANA, CA 19-9, epithelial tumour antigen (ETA), BCMA, cancer testis antigens such as CTA New York (o) esophageal squamous cell carcinoma (NYESO) and glycoprotein 100 (GP100), preferentially expressed antigen in melanoma (PRAME), collagen type IV alpha 3 chain (COL6A3), MR1, CD1c, human epidermal growth factor receptor 2 (HER2), solute carrier family 3 member 2 (SLC3A2) and avb6 integrin.

In some embodiments, the cancer antigen is selected from NYESO, GP100, PRAME, COL6A3, MR1, CD1c, HER2, SLCA2, CD19, PSMA, AFP, CEA, CA-125, MUC1, ETA, tyrosinase and MAGE. In some embodiments, the CAR is an anti-CD19, anti-SLC3A2 or anti-PSMA CAR.

In some embodiments, the CAR is an anti-CD19 or anti-PSMA CAR.

MAGE may be selected from MAGE A1, MAGE A2, MAGE A4 or MAGE A8.

The CAR may be linked to a reporter protein, for example GFP, MYC epitope flag or a FLAG epitope tag. Other suitable reporter proteins will be known to those skilled in the art. In some embodiments, the CAR comprises a second-generation CAR.

Suitable CAR intracellular signalling domains may include any suitable signalling domain, including any region comprising an Immune-receptor-Tyrosine-based-Activation-Motif (ITAM), as reviewed for example by Love et al. Cold Spring Harbor Perspect. Biol 2010 2 (6)|a002485. In some embodiments, the signalling domain comprises the intracellular domain of human CD3 [zeta] chain as described for example in U.S. Pat. No. 7,446,190, or a variant thereof.

Various co-stimulatory domains are known to engineer CAR cells. The CAR may comprise one or more of these domains. Suitable co-stimulatory domains include, but are not necessarily limited to members of the B7/CD28 family such as B7-1, B7-2, B7-H1, B7-H2, B7-H3, B7-H4, B7-H6, B7-H7, BTLA, CD28, CTLA-4, Gi24, ICOS, PD-1, PD-L2 or PDCD6; or ILT/CD85 family proteins such as LILRA3, LILRA4, LILRB1, LILRB2, LILRB3 or LILRB4; or tumour necrosis factor (TNF) superfamily members such as 4-1BB, BAFF, BAFF R, CD27, CD30, CD40, DR3, GITR, HVEM, LIGHT, Lymphotoxin-alpha, OX40, RELT, TACI, TL1A, TNF-alpha or TNF RII; or members of the SLAM family such as 2B4, BLAME, CD2, CD2F-10, CD48, CD58, CD84, CD229, CRACC, NTB-A or SLAM; or members of the TIM family such as TIM-1, TIM-3 or TIM-4; or other co-stimulatory molecules such as CD7, CD96, CD160, CD200, CD300a, CRTAM, DAP12, Dectin-1, DPPIV, EphB6, Integrin alpha 4 beta 1, Integrin alpha 4 beta 7/LPAM-1, LAG-3 or TSLP R.

In some embodiments the immunotherapeutic molecule comprises a MHC Class II cell surface receptor. Preferably, the MHC Class II cell surface receptor comprises or consists of HLA-DR. Without wishing to be bound by theory, exogenous expression of HLA-DR by the ILCs may aid immunoregulatory activity.

In some embodiments the immunotherapeutic molecule comprises a cytokine. The cytokine may be an immunoregulatory cytokine, for example IL-10 or TGF-β. The expression of an immunoregulatory cytokine or receptor in the in vitro population of the ILCs may have particular use when the ILCs are used for the treatment of an autoimmune disease, for example inflammatory bowel disease (IBD) or multiple sclerosis, or an allergy. Further autoimmune diseases and specific allergies are described below.

In other embodiments, the cytokine is an inflammatory cytokine. Exemplary inflammatory cytokines include, but are not necessarily limited to IL-22, IL-17A, IL-5, IL-4, Amphiregulin, IFN-γ, IL-2, IL-1, IL-18, TNF-α and GM-CSF. Thus, in some embodiments the cytokine comprises one or more of IL-22, IL-17A, IL-5, IL-4, Amphiregulin, IFN-γ, IL-2, IL-1, IL-18, TNF-α and GM-CSF. In some embodiments the cytokine comprises one or more of IL-22, IL-17A, IL-5, IL-4, IFN-γ, TNF-α and GM-CSF. The expression of such an inflammatory cytokine may have particular use when the ILCs are used for the treatment of a cancer.

Various chemokine receptors are known in the art. Exemplary chemokine receptors include, but are not necessarily limited to CXC chemokine receptors, CC chemokine receptors, XCR1 and CX3CR1.

In some embodiments the chemokine receptor comprises a CC chemokine receptor. For example, the chemokine receptor may comprise one or more of CCR1, CCR2, CCR3, CCR4, CCR5, CCR6, CCR7, CCR8, CCR9, CCR10 and CCR11. In some embodiments, the chemokine receptor comprises or consists of CCR7 or CCR3. In some embodiments the chemokine receptor comprises or consists of CCR7.

Without wishing to be bound by theory, the present inventors believe that the expression of a chemokine receptor in the in vitro population of ILCs may assist with tissue-specific therapeutic targeting of the ILCs. This may further increase the therapeutic efficacy of the in vitro population of ILCs.

Preferably, a vector comprises the exogenous polynucleotide. The vector may be viral or non-viral. Various viral and non-viral vectors are known to those skilled in the art. Non-viral vectors include plasmids, episomal vectors, and human artificial chromosomes (see, e.g., Harrington et al., 1997, Nat Genet. 15:345). For example, non-viral vectors useful for expression of the exogenous polypeptide in mammalian (e.g., human) cells include pThioHis A, B and C, pcDNA3.1/His, pEBVHis A, B and C, (Invitrogen, San Diego, Calif.), MPS V vectors, and numerous other vectors known in the art for expressing other proteins. Useful viral vectors include vectors based on retroviruses, adenoviruses, adeno-associated viruses, herpes viruses, vectors based on SV40, papilloma virus, HBP Epstein Barr virus, vaccinia virus vectors and Semliki Forest virus (SFV). See, Brent et al., supra; Smith, 1995, Annu. Rev. Microbiol. 49:807; and Rosenfeld et al., 1992, Cell 68:143. In particular, retroviral, lentiviral, adenoviral or adeno-associated viral vectors are commonly used for expression in immune cells such as T-cells. Examples of such vectors include the SFG retroviral expression vector (see Riviere et al., 1995, Proc. Natl. Acad. Sci. (USA) 92:6733-6737).

In some embodiments, the vector is a retroviral or lentiviral vector. Optionally, the vector is an SFG retroviral vector. In some embodiments the vector is a lentiviral vector. Lentiviral vectors include self-inactivating lentiviral vectors (so-called SIN vectors).

The choice of vector depends on the intended host cells in which the vector is to be expressed. Expression vectors for mammalian host cells can include expression control sequences, such as an origin of replication, a promoter, and an enhancer (see, e.g., Queen, et al., 1986, Immunol. Rev. 89:49-68), and necessary processing information sites, such as ribosome binding sites, RNA splice sites, polyadenylation sites, and transcriptional terminator sequences. These vectors usually contain promoters derived from mammalian genes or from mammalian viruses. Suitable promoters may be constitutive, cell type-specific, stage-specific, and/or modulatable or regulatable. Useful promoters include, but are not limited to, the metallothionein promoter, the constitutive adenovirus major late promoter, the dexamethasone-inducible MMTV promoter, the SV40 promoter, the MRP polIII promoter, the constitutive MPS V promoter, the tetracycline-inducible CMV promoter (such as the human immediate-early CMV promoter), the constitutive CMV promoter, the EF1 alpha promoter, the phosphoglycerate kinase (PGK) promoter and promoter-enhancer combinations known in the art.

The vector may further comprise a polynucleotide encoding a reporter gene. Suitable reporter genes include, but are not necessarily limited to HNIS, hNET and HSVtK.

Cultures of transformed organisms can be expanded under non-inducing conditions without biasing the population for coding sequences whose expression products are better tolerated by the in vitro population of ILCs. In addition to promoters, other regulatory elements may also be required or desired for efficient expression. These elements typically include an ATG initiation codon and adjacent ribosome binding site or other sequences. In addition, the efficiency of expression may be enhanced by the inclusion of enhancers appropriate to the cell system in use (see, e.g., Scharf et al., 1994, Results Probl. Cell Differ. 20:125; and Bittner et al., 1987, Meth. Enzymol., 153:516). For example, the SV40 enhancer or CMV enhancer may be used to increase expression in mammalian host cells.

The genetic engineering of immune cells such as ILCs can be carried out according to standard cloning and expression techniques, which are known in the art (e.g., as described in Sambrook, J., Fritsh, E. F., and Maniatis, T. Molecular Cloning: A Laboratory Manual 2nd, ed., Cold Spring Harbor Laboratory, Cold Spring Harbor Laboratory Press, Cold Spring Harbor, N.Y., 1989). The vector may be introduced into the in vitro population of ILCs using such techniques. Introduction may comprise transfection or transduction into the in vitro population of ILCs.

The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like.

As used herein, the term polynucleotide refers to a polymer comprising two or more nucleotides. Preferably, the polynucleotide comprises at least 30 nucleotides, at least 40 nucleotides, at least 50 nucleotides or at least 100 nucleotides. The nucleotides can be naturally occurring or artificial.

A nucleotide typically contains a nucleobase, a sugar and at least one linking group, such as a phosphate, 2′O-methyl, 2′ methoxy-ethyl, phosphoramidate, methylphosphonate or phosphorothioate group. The nucleobase is typically heterocyclic. Nucleobases include, but are not limited to, purines and pyrimidines and more specifically adenine (A), guanine (G), thymine (T), uracil (U) and cytosine (C). The sugar is typically a pentose sugar. Nucleotide sugars include, but are not limited to, ribose and deoxyribose. The sugar and the nucleobase together form a nucleoside. Preferred nucleosides include, but are not limited to, adenosine, guanosine, 5-methyluridine, uridine, cytidine, deoxyadenosine, deoxyguanosine, thymidine, deoxyuridine and deoxycytidine. The nucleosides may be adenosine, guanosine, uridine and cytidine.

The nucleotides are typically ribonucleotides or deoxyribonucleotides. The nucleotides may be deoxyribonucleotides. The nucleotides typically contain a monophosphate, diphosphate or triphosphate. Phosphates may be attached on the 5′ or 3′ side of a nucleotide. Nucleotides include, but are not limited to, adenosine monophosphate (AMP), adenosine diphosphate (ADP), adenosine triphosphate (ATP), guanosine monophosphate (GMP), guanosine diphosphate (GDP), guanosine triphosphate (GTP), thymidine monophosphate (TMP), thymidine diphosphate (TDP), thymidine triphosphate (TTP), uridine monophosphate (UMP), uridine diphosphate (UDP), uridine triphosphate (UTP), cytidine monophosphate (CMP), cytidine diphosphate (CDP), cytidine triphosphate (CTP), 5-methylcytidine monophosphate, 5-methylcytidine diphosphate, 5-methylcytidine triphosphate, 5-hydroxymethylcytidine monophosphate, 5-hydroxymethylcytidine diphosphate, 5-hydroxymethylcytidine triphosphate, cyclic adenosine monophosphate (CAMP), cyclic guanosine monophosphate (CGMP), deoxyadenosine monophosphate (dAMP), deoxyadenosine diphosphate (dADP), deoxyadenosine triphosphate (dATP), deoxyguanosine monophosphate (dGMP), deoxyguanosine diphosphate (dGDP), deoxyguanosine triphosphate (dGTP), deoxythymidine monophosphate (dTMP), deoxythymidine diphosphate (dTDP), deoxythymidine triphosphate (dTTP), deoxyuridine monophosphate (dUMP), deoxyuridine diphosphate (dUDP), deoxyuridine triphosphate (dUTP), deoxycytidine monophosphate (dCMP), deoxycytidine diphosphate (dCDP) and deoxycytidine triphosphate (dCTP), 5-methyl-2′-deoxycytidine monophosphate, 5-methyl-2′-deoxycytidine diphosphate, 5-methyl-2′-deoxycytidine triphosphate, 5-hydroxymethyl-2′-deoxycytidine monophosphate, 5-hydroxymethyl-2′-deoxycytidine diphosphate and 5-hydroxymethyl-2′-deoxycytidine triphosphate. The nucleotides may be selected from AMP, UMP, GMP, CMP, dAMP, dTMP, dGMP or dCMP. In some embodiments, the nucleotides are selected from dAMP, dTMP, dGMP or dCMP.

The nucleotides may contain additional modifications. In particular, suitable modified nucleotides include, but are not limited to, 2′amino pyrimidines (such as 2′-amino cytidine and 2′-amino uridine), 2′-hydroxyl purines (such as, 2′-fluoro pyrimidines (such as 2′-fluorocytidine and 2′fluoro uridine), hydroxyl pyrimidines (such as 5′-α-P-borano uridine), 2′-O-methyl nucleotides (such as 2′-O-methyl adenosine, 2′-O-methyl guanosine, 2′-O-methyl cytidine and 2′-O-methyl uridine), 4′-thio pyrimidines (such as 4′-thio uridine and 4′-thio cytidine) and nucleotides have modifications of the nucleobase (such as 5-pentynyl-2′-deoxy uridine, 5-(3-aminopropyl)-uridine and 1,6-diaminohexyl-N-5-carbamoylmethyl uridine).

One or more nucleotides in the polynucleotide may be modified, for instance with a label or a tag. The label may be any suitable label which allows the nucleotides to be detected. Suitable labels include, but are not limited to, fluorescent molecules, radioisotopes, e.g. ¹²⁵I, ³⁵S, enzymes, antibodies, antigens, other polynucleotides and ligands such as biotin.

The nucleotides in the exogenous polynucleotide may be attached to each other in any manner. The nucleotides may be linked by phosphate, 2′O-methyl, 2′ methoxy-ethyl, phosphoramidate, methylphosphonate or phosphorothioate linkages. The nucleotides are typically attached by their sugar and phosphate groups. The nucleotides may be connected via their nucleobases as in pyrimidine dimers.

The exogenous polynucleotide may comprise a deoxyribonucleic acid (DNA) or a ribonucleic acid (RNA). Preferably, the exogenous polynucleotide comprises DNA. The exogenous polynucleotide may be any synthetic nucleic acid known in the art, such as peptide nucleic acid (PNA), glycerol nucleic acid (GNA), threose nucleic acid (TNA), locked nucleic acid (LNA), morpholino nucleic acid or other synthetic polymers with nucleotide side chains.

Substitutions may be used for the practices of codon optimisation and codon wobble, both of which are known to those skilled in the art. Thus, it will be appreciated that codon-optimised and codon-wobbled exogenous polynucleotide are also envisaged. In some embodiments, the exogenous polynucleotide is codon-optimised for human expression.

The exogenous polynucleotide can be produced by de novo solid-phase DNA synthesis or by PCR mutagenesis of an existing sequence. Direct chemical synthesis of polynucleotides can be accomplished by methods known in the art, such as the phosphotriester method of Narang et al., 1979, Meth. Enzymol. 68:90; the phosphodiester method of Brown et al., 1979, Meth. Enzymol. 68:109; the diethylphosphoramidite method of Beaucage et al., 1981, Tetra. Lett., 22:1859; and the solid support method of U.S. Pat. No. 4,458,066. Introducing mutations to a polynucleotide sequence by PCR can be performed as described in, e.g., PCR Technology: Principles and Applications for DNA Amplification, H. A. Erlich (Ed.), Freeman Press, NY, N.Y., 1992; PCR Protocols: A Guide to Methods and Applications, Innis et al. (Ed.), Academic Press, San Diego, Calif, 1990; Mattila et al., 1991, Nucleic Acids Res. 19:967; and Eckert et al., 1991, PCR Methods and Applications 1:17.

The invention also provides a pharmaceutical composition comprising the immune cells obtainable by the method of the present invention or the in vitro population as defined above and a pharmaceutically or physiologically acceptable diluent and/or carrier. The invention also provides a pharmaceutical composition comprising a human ILCreg of the invention or an in vitro population of human ILCregs of the invention and a pharmaceutically or physiologically acceptable diluent and/or carrier.

The carrier and/or diluent is generally selected to be suitable for the intended mode of administration and can include agents for modifying, maintaining, or preserving, for example, the pH, osmolarity, viscosity, clarity, colour, isotonicity, odour, sterility, stability, rate of dissolution or release, adsorption, or penetration of the composition. Typically, these carriers and/or diluents include aqueous or alcoholic/aqueous solutions, emulsions, or suspensions, including saline and/or buffered media.

Suitable further agents for inclusion in the pharmaceutical compositions include, but are not limited to, amino acids (such as glycine, glutamine, asparagine, arginine, or lysine), antimicrobials, antioxidants (such as ascorbic acid, sodium sulphite, or sodium hydrogen-sulphite), buffers (such as borate, bicarbonate, Tris-HCl, citrates, phosphates, or other organic acids), bulking agents (such as mannitol or glycine), chelating agents (such as ethylenediamine tetraacetic acid (EDTA)), complexing agents (such as caffeine, polyvinylpyrrolidone, beta-cyclodextrin, or hydroxypropyl-beta-cyclodextrin), fillers, monosaccharides, disaccharides, and other carbohydrates (such as glucose, mannose, or dextrins), proteins (such as free serum albumin, gelatin, or immunoglobulins), colouring, flavouring and diluting agents, emulsifying agents, hydrophilic polymers (such as polyvinylpyrrolidone), low molecular weight polypeptides, salt-forming counterions (such as sodium), preservatives (such as benzalkonium chloride, benzoic acid, salicylic acid, thimerosal, phenethyl alcohol, methylparaben, propylparaben, chlorhexidine, sorbic acid, or hydrogen peroxide), solvents (such as glycerin, propylene glycol, or polyethylene glycol), sugar alcohols (such as mannitol or sorbitol), suspending agents, surfactants or wetting agents (such as pluronics; PEG; sorbitan esters; polysorbates such as Polysorbate 20 or

Polysorbate 80; Triton; tromethamine; lecithin; cholesterol or tyloxapal), stability enhancing agents (such as sucrose or sorbitol), tonicity enhancing agents (such as alkali metal halides, such as sodium or potassium chloride, or mannitol sorbitol), delivery vehicles, excipients and/or pharmaceutical adjuvants.

The carrier and/or diluent may be a parenteral, optionally intravenous vehicle. Suitable parenteral vehicles include sodium chloride solution, Ringer's dextrose, dextrose and sodium chloride and lactated Ringer's. Suitable physiologically-acceptable thickeners such as carboxymethylcellulose, polyvinylpyrrolidone, gelatin and alginates may be included. Intravenous vehicles include fluid and nutrient replenishers and electrolyte replenishers, such as those based on Ringer's dextrose. In some cases, one might include agents to adjust tonicity of the composition, for example, sugars, polyalcohols such as mannitol, sorbitol, or sodium chloride in a pharmaceutical composition. For example, in many cases it is desirable that the composition is substantially isotonic. Preservatives and other additives, such as antimicrobials, antioxidants, chelating agents, and inert gases, may also be present. The precise formulation will depend on the route of administration. Additional relevant principle, methods and components for pharmaceutical formulations are well known (see, e.g., Allen, Loyd V. Ed, (2012) Remington's Pharmaceutical Sciences, 22^{nd} Edition).

A pharmaceutical composition of the present invention can be administered by one or more routes of administration using one or more of a variety of methods known in the art. As will be appreciated by the skilled person, the route and/or mode of administration will vary depending upon the desired results. Routes of administration for pharmaceutical compositions of the invention include intravenous, intramuscular, intradermal, intraperitoneal, intrapleural, subcutaneous, intratumoural, spinal, or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural, intratumoural, intrapleural and intra-sternal injection and infusion. In some embodiments, the pharmaceutical composition is administered intratumourally. In other embodiments, administration is intrapleural or intraperitoneal. When parenteral administration is contemplated, the pharmaceutical compositions are usually in the form of a sterile, pyrogen-free, parenterally acceptable composition. A particularly suitable vehicle for parenteral injection is a sterile, isotonic solution, properly preserved. The pharmaceutical composition can be in the form of a lyophilizate, such as a lyophilized cake.

Alternatively, the pharmaceutical composition of the invention can be administered by a non-parenteral route, such as a topical, epidermal, or mucosal route of administration, for example, intranasally, orally, vaginally, rectally, sublingually, or topically.

In some embodiments, the pharmaceutical composition is for subcutaneous administration. Typically, the pharmaceutical compositions for subcutaneous administration contain suitable stabilizers (e.g., amino acids, such as methionine, and or saccharides such as sucrose), buffering agents and tonicifying agents. Alternatively, the pharmaceutical composition may be for intravenous administration.

The invention also provides a kit comprising the immune cells obtainable by the method of the present invention, the in vitro population and/or the pharmaceutical composition as defined above. The kit may further comprise instructions for use. In some embodiments, the immune cells obtainable by the method of the present invention, the in vitro population and/or the pharmaceutical composition is provided in an aqueous solution, optionally buffered solution and/or at a temperature of at least −20° C.

Also provided is a method of treating or preventing a disease in a subject, wherein the method comprises administering to the subject the immune cells, the in vitro population and/or the pharmaceutical composition of the invention.

In some embodiments, the immune cells are lymphoid cells. Preferably, the immune cells are T-cells, B-cells and/or innate lymphoid cells (ILC). More preferably, the immune cells are ILCs. The in vitro population may comprise Group 1 ILCs. In some embodiments, the in vitro population comprises ILC1 cells and/or NK cells. In some embodiments, the in vitro population comprises or consists of NK cells. The NK cells are preferably cytotoxic NK cells. This is shown in Example 9. The ILCs are preferably ILCregs, murine ILCregs or human ILCregs of the invention. The in vitro population preferably comprises ILCregs, murine ILCregs or human ILCregs of the invention. The in vitro population may be a heterogenous population. Alternatively, the in vitro population may be a homogenous population.

The method typically comprises administering a therapeutically effective amount or a prophylactically effective amount of the immune cells, the in vitro population and/or the pharmaceutical composition of the invention. A therapeutically effective amount is an amount which ameliorates one or more symptoms, such as all the symptoms, of the disease and/or abolishes one or more symptoms, such as all the symptoms, of the disease. The therapeutically effective amount preferably cures the disease. A prophylactically effective amount is an amount which prevents the onset of the disease and/or prevents the onset of one or more symptoms, such as all the symptoms, of the disease. The prophylactically effective amount preferably prevents the subject from developing the disease. Suitable amounts are discussed in more detail below.

The immune cells, the in vitro population and/or the pharmaceutical composition of the invention may be administered to a subject that displays symptoms of disease. The the immune cells, the in vitro population and/or the pharmaceutical composition of the invention may be administered to a subject that is asymptomatic, i.e. does not display symptoms of disease. The immune cells, the in vitro population and/or the pharmaceutical composition of the invention may be administered when the subject's disease status is unknown or the patient is expected not to have a disease. The the immune cells, the in vitro population and/or the pharmaceutical composition of the invention may be administered to a subject that is predisposed, such as genetically predisposed, to developing the disease.

The subject may be a mammal. Optionally, the subject is a human, horse, dog or cat. In some embodiments, the subject is human. Alternatively, the subject may be a horse.

Various diseases are suitable for treatment or prophylaxis by administration of the immune cells, the in vitro population and/or the pharmaceutical composition of the invention. Any disease which can be treated or prevented using immunotherapy is envisaged.

For example, the disease may be cancer, an infection, an autoimmune disease or an allergy. In some embodiments the disease is cancer, an autoimmune disease or an allergy. In some embodiments the disease is a cancer or an autoimmune disease. In other embodiments the disease is an autoimmune disease or an allergy. Alternatively, the disease is an allergy or cancer. In some embodiments the disease is a cancer. In some embodiments the disease is an autoimmune disease. In other embodiments the disease is an allergy.

In some embodiments the disease is an inflammatory disease. Such diseases are discussed in more detail below. In some embodiments the disease comprises a chronic or acute inflammatory disease. The chronic or acute inflammatory disease may comprise a chronic or acute infection.

The autoimmune disease may include, but not necessarily be limited to inflammatory bowel disease, eczema, rheumatoid arthritis, psoriasis, multiple sclerosis (MS), myasthenia gravis, type 1 diabetes mellitus, systemic lupus erythematosus (SLE or Lupus), Guillain-Barre syndrome, chronic inflammatory demyelinating polyneuropathy, Graves' disease, Hashimoto's thyroiditis and vasculitis.

In some embodiments, the autoimmune disease is selected from inflammatory bowel disease, rheumatoid arthritis, psoriasis, multiple sclerosis (MS), type 1 diabetes mellitus and systemic lupus erythematosus (SLE or Lupus). Additional autoimmune diseases are discussed below. In some embodiments the autoimmune disease comprises inflammatory bowel disease. Exemplary inflammatory bowel diseases include Crohn's disease and ulcerative colitis.

The cancer may include, but not necessarily be limited to, a solid tumour cancer, a soft tissue tumour, a metastatic lesion, and a haematological cancer. For example, the cancer can be liver cancer, lung cancer, breast cancer, prostate cancer, lymphoid cancer, colon cancer, renal cancer, bone cancer, pancreatic cancer, skin cancer, cancer of the head or neck, such as squamous cell carcinoma of the head and neck (SCCHN), cutaneous or intraocular malignant melanoma, uterine cancer, ovarian cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, Hodgkin's Disease, non-Hodgkin's lymphoma, cancer of the oesophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, chronic or acute leukaemias including acute myeloid leukaemia, chronic myeloid leukaemia, acute lymphoblastic leukaemia, chronic lymphocytic leukaemia, solid tumours of childhood, lymphocytic lymphoma, cancer of the bladder, cancer of the kidney or ureter, carcinoma of the renal pelvis, neoplasm of the central nervous system (CNS), primary CNS lymphoma, tumour angiogenesis, spinal axis tumour, brain stem glioma, pituitary adenoma, Kaposi's sarcoma, epidermoid cancer, squamous cell cancer, T-cell lymphoma, myelodysplastic syndrome (MDS), chronic myelogenous leukaemia-chronic phase (CMLCP), diffuse large B-cell lymphoma (DLBCL), cutaneous T-cell lymphoma (CTCL), peripheral T-cell lymphoma (PTCL), hepatocellular carcinoma (HCC), gastrointestinal stromal tumours (GIST), non-small cell lung carcinoma (NSCLC), cutaneous melanoma, mucosal melanoma, cutaneous squamous cell carcinoma (CSCC), small-cell lung cancer, squamous cell carcinoma of the lung, Merkle cell carcinoma, environmentally induced cancers including those induced by asbestos, and combinations of said cancers. In some embodiments, the cancer is selected from the above group.

The cancer may be a solid tumour cancer.

In some embodiments, the cancer is selected from the group consisting of cancer of the head and/or neck, ovarian cancer, malignant mesothelioma, breast cancer, pancreatic cancer, colorectal cancer, lung cancer, gastric cancer, bladder cancer, prostate cancer, oesophageal cancer, endometrial cancer, hepatobiliary cancer, chronic or acute leukaemia including acute myeloid leukaemia, duodenal carcinoma, thyroid carcinoma, cancer of the central nervous system or renal cell carcinoma.

In some embodiments, the cancer is selected from ovarian cancer, breast cancer, optionally triple-negative breast cancer, pancreatic cancer, chronic or acute leukaemia including acute myeloid leukaemia, malignant mesothelioma, and combinations of said cancers.

The subject may have been pre-treated with a chemotherapeutic agent. In some embodiments, the disease is cancer or autoimmune disease and the subject has been pre-treated with a chemotherapeutic agent.

When the disease is cancer, the administration of the immune cells, the in vitro population and/or the pharmaceutical composition of the invention to the subject may result in a decrease in tumour size of about 10%, about 20%, about 30%, about 40%, about 50%, about 60%, about 70%, about 80%, about 90%, or even about 100%, when compared to an untreated tumour.

The allergy may include, but not necessarily be limited to allergic rhinitis (which may otherwise be referred to as hayfever), dust mite allergy, animal allergy, food allergy, insect bite/sting allergy, medicinal allergy, latex allergy, mould allergy, allogeneic rejection and/or graft versus host disease.

Common food allergies include, but are not necessarily limited to nut allergy, fruit allergy, shellfish allergy, cow's milk protein allergy, egg allergy and a lactose allergy. The nut allergy may be a peanut allergy. The fruit allergy may be a strawberry, rhubarb, pineapple, apple or pear allergy.

In some embodiments the allergy is selected from allergic rhinitis, food allergy, allogeneic rejection and graft versus host disease.

Also provided is a method of treating or preventing a disease in a subject, wherein the method comprises administering to the subject a human ILCreg of the invention, an in vitro population of human ILCregs of the invention and/or a pharmaceutical composition of the invention comprising human ILCregs of the invention. The discussion above relating to the amounts of cells and subjects equally applies to this method. The disease is preferably an inflammatory disease, such as an autoimmune disease, an infection or cancer. The inflammatory disease may be chronic or acute as discussed above. The inflammatory disease may be present in the cells of any of the tissues discussed above, including skin, gastro-intestinal, lung, thymic, thyroid, reproductive, bladder, kidney, pancreas, or liver tissue. An inflammatory disease is a disease or infection which comprises the damage or destruction of healthy viable cells. Examples of inflammatory diseases include, but not limited to, an autoimmune disease, an allergy, asthma, coeliac disease, nephritis, hepatitis, reperfusion injury, graft versus host disease (GvHD), transplant rejection and an infection. By “infection”, this will be understood to infection with a pathogen. In some embodiments, the inflammatory disease comprises an autoimmune disease, an infection, or cancer.

An autoimmune disease may comprise rheumatoid arthritis, psoriasis, system lupus erythematosus (lupus), inflammatory bowel disease, multiple sclerosis, diabetes, Guillain-Barre syndrome, chronic inflammatory demyelinating polyneuropathy, Graves' disease, Hashimoto's thyroiditis, Myasthenia gravis, Aplastic Anaemia (AA), Vasculitis or combinations thereof. The autoimmune disease preferably comprises inflammatory bowel disease. Exemplary inflammatory bowel diseases include Crohn's disease and ulcerative colitis.

The infection may be an infection with any pathogen. The pathogen may be a bacterium, an archaeon, a single cell eukaryote, such as an amoeba or a paramecium, a fungus, or a virus.

The bacterium may be Gram negative or Gram positive. The Gram-positive bacterium is preferably from the genus Bacillus, Clostridium, Enterococcus, Mycobacterium, Staphylococcus or Streptococcus. The Gram-positive bacterium may be from the genus Pasteurella or Nocardia.

The Gram negative bacterium is preferably from the genus Aggregatibacter, Bacteroides, Bartonella, Brucella, Campylobacter, Chylamidia, Enterbacter, Francisella, Haemophilus, Heliobacter, Klebsiella, Legionella, Moraxella, Neisseria, Porphyromonas, Pseudomonas, Salmonella, Serratia, Stenotrophomonas, Vibrio or Yersinia. The Gram negative bacterium may be from the genus Escherichia or Pseudomonas.

The bacterium may be from the genus Borrelia, Chlamydophila, Listeria, Mycoplasma, Proteus or Treponema. The bacterium is preferably Aggregatibacter actinomycetemcomitans, Bacillus anthracis, Bacillus licheniformis, Bacteroides fragilis, Bartonella henselae, Bordetella pertussis, Borrelia burgdorferi, Brucella abortus, Campylobacter jejuni, Chlamydia trachomatis, Chlamydophila pneumoniae, Clostridium difficile, Clostridium perfringens, Enterobacter aerogenes, Enterococcus faecalis, Enterococcus faecium, Francisella tularensis, Haemophilus influenzae, Helicobacter pylori, Klebsiella oxytoca, Legionella pneumophila, Listeria monocytogenes, Moraxella catarrhalis, Mycobacterium avium, Mycobacterium bovis, Mycoplasma genitalium, Mycoplasma pneumoniae, Neisseria gonorrhoeae, Neisseria meningitidis, Porphyromonas gingivalis, Proteus mirabilis, Pseudomonas aeruginosa, Salmonella enterica, Serratia marcescens, Staphylococcus aureus, Staphylococcus epidermidis, Staphylococcus haemolyticus, Stenotrophomonas maltophilia, Streptococcus mutans, Streptococcus pyogenes, Streptococcus salivarius, Streptococcus sanguinis, Treponema pallidum, Vibrio cholera, Vibrio parahaemolyticus or Yersinia enter ocolitica.

Other specific examples of bacteria include, but are not limited, to Mycobacterium tuberculosis, Mycobacterium intracellilare, Mycobacterium kansaii, Mycobacterium gordonae, Streptococcus agalactiae, Streptococcus viridans group, Streptococcus faecalis, Streptococcus bovis, Streptococcus pneumoniae, Corynebacterium diptheriae, Erysipelothrix rhusiopathie, Clostridium tetani, Klebsiella pneumoniae, Pasteurella multocida, Fusobacterium nucleatum, Streptobacillus moniliformis, Treponema pertenue and Actinomyces israelii.

The microbe is preferably a Mycobacterium species that are capable of causing tuberculosis. The microbe is preferably Mycobacterium tuberculosis (M. tuberculosis), Mycobacterium africanum (M. africanum), Mycobacterium orygis (M. orygis, which may otherwise be referred to as the oryx bacilli), Mycobacterium bovis (M. bovis), Mycobacterium microti (M. microti), Mycobacterium canetti (M. canetti), Mycobacterium caprae (M. caprae), Mycobacterium pinnipedii (M. pinnipedii), Mycobacterium suricattae (M. suricattae) or Mycobacterium mungi (M. mungi)

The fungus is preferably from the genus Absidia, Acremonium, Aspergillus, Aureobasidium, Basidiobolus, Blastomyces, Blastoschizomyces, Candida, Cladosporium, Coccidioides, Cryptococcus, Cunninghamella, Curvularia, Debaryomyces, Exophiala, Exserohilum, Fonsecea, Fusarium, Geotrichum, Histoplasma, Issatchenkia, Kluyveromyces, Malezzesia, Mucor, Paracoccidioides, Paecilomyces, Penicillium, Pichia, Pneumocystis, Rhizomucor, Rhizopus, Rhodotorula, Saccharomyces, Scedosporium, Schizophyllum, Scopulariopsis, Sporothrix, Trichoderma, Trichophyton or Trichosporon.

The fungus is preferably Aspergillus fumigatus, Aspergillus flavus, Aspergillus lentulus, Aspergillus terreus, Aspergillus nidulans, Aspergillus oryzae, Aspergillus niger, Candida albicans, Candida caribbica (Candida fermentati), Candida dubliniensis, Candida famata (Debaryomyces hansenii), Candida fukuyamaensis (Candida xestobii or Candida carpophila), Candida guilliermondii, Candida kefyr (Kluyveromyces marxianus), Candida krusei (Issatchenkia orientalis), Candida metapsilosis, Candida orthopsilosis, Candida parapsilosis, Candida parapsilosis, Candida pelliculosa, Candida psychrophila, Candida rugosa, Candida smithsonii, Candida tropicalis, Candida utilis, Coccidioides immitis, Cryptococcus bacillisporus, Cryptococcus gattii, Cryptococcus grubii, Cryptococcus neoformans, Debaryomyces coudertii, Debaryomyces maramus, Debaryomyces nepalensis, Debaryomyces prosopidis, Debaryomyces robertsiae, Debaryomyces udenii, Histoplasma capsulatum, Kluyveromyces lactis, Pichia cecembensis, Rhodotorula araucariae, Rhodotorula babjevae, Rhodotorula dairensis, Rhodotorula diobovatum, Rhodotorula glutinis, Rhodotorula kratochvilovae, Rhodotorula paludigenum, Rhodotorula sphaerocarpum, Rhodotorula toruloides, Rhodotorula mucliaginosa, Saccharomyces ‘sensu stricto’, Saccharomyces bayanus, Saccharomyces boulardii, Saccharomyces cariocanus, Saccharomyces kudiavzevii, Saccharomyces mikatae, Saccharomyces paradioxus, Saccharomyces pastorianus, Saccharomyces uvarum, Saccharomyces cerevisiae or Tsuchiyaea wingfieldii.

The virus may belong to the family Retroviridae, such as human deficiency viruses, such as HIV-I (also referred to as HTLV-III), HIV-II, LAC, IDLV-III/LAV, HIV-III or other isolates such as HIV-LP, the family Picornaviridae, such as poliovirus, hepatitis A, enteroviruses, human Coxsackie viruses, rhinoviruses, echoviruses, the family Calciviridae, such as viruses that cause gastroenteritis, the family Togaviridae, such as equine encephalitis viruses and rubella viruses, the family Flaviviridae, such as dengue viruses, encephalitis viruses and yellow fever viruses, the family Coronaviridae, such as coronaviruses (e.g., SARS-COV or SARS-COV-2/COVID-19), the family Rhabdoviridae, such as vesicular stomata viruses and rabies viruses, the family Filoviridae, such as Ebola viruses, the family Paramyxoviridae, such as parainfluenza viruses, mumps viruses, measles virus and respiratory syncytial virus, the family Orthomyxoviridae, such as influenza viruses, the family Bungaviridae, such as Hataan viruses, bunga viruses, phleoboviruses and Nairo viruses, the family Arena viridae, such as hemorrhagic fever viruses, the family Reoviridae, such as reoviruses, orbiviruses and rotaviruses, the family Bimaviridae, the family Hepadnaviridae, such as hepatitis B virus, the family Parvoviridae, such as parvoviruses, the Papovaviridae, such as papilloma viruses and polyoma viruses, the family Adenoviridae, such as adenoviruses, the family Herpesviridae, such as herpes simplex virus (HSV) I and II, varicella zoster virus and pox viruses, or the family Iridoviridae, such as African swine fever virus). The virus may be an unclassified virus, such as the etiologic agents of Spongiform encephalopathies, the agent of delta hepatitis, the agents of non-A, non-B hepatitis (class 1 enterally transmitted; class 2 parenterally transmitted such as Hepatitis C); Norwalk and related viruses and astroviruses.

The cancer may be any of those discussed above.

Administration of the human ILCregs of the invention, including in the population or pharmaceutical composition of the invention, to the subject may reduce symptoms by at least about 10%, about 20%, about 30%, about 40%, about 50%, about 60%, about 70%, about 80%, about 90%, or even about 100%, when compared to an untreated subject.

In embodiments administering immune cells or the in vitro population, the number of cells administered to the subject should take into account the route of administration, the disease being treated, the weight of the subject and/or the age of the subject. In general, from about 1×10⁶ to about 1×10¹¹ immune cells are administered to the subject. In some embodiments, from about 1×10⁷ to about 1×10¹⁰ immune cells, or from about 1×10⁸ to about 1×10⁹ immune cells are administered to the subject. This also applies to the embodiments involving the human ILCregs of the invention.

The invention also provides the immune cells, in vitro population and/or the pharmaceutical composition of the invention for use in any of the therapeutic methods described above. Thus, also provided is immune cells, the in vitro population and/or the pharmaceutical composition of the invention for use in the treatment or prevention of a disease. This may otherwise be referred to for use in therapy. In particular, the invention provides the immune cells, the in vitro population and/or the pharmaceutical composition of the invention for use in the treatment or prevention of cancer, autoimmune disease or allergy. Preferably, the invention provides the immune cells, the in vitro population and/or the pharmaceutical composition of the invention for use in the treatment or prevention of cancer or autoimmune disease.

The invention also provides a human ILCreg of the invention, an in vitro population of human ILCregs of the invention and/or a pharmaceutical composition of the invention comprising human ILCregs of the invention for use in any of the therapeutic methods described above. Thus, also provided is a human ILCreg of the invention, an in vitro population of human ILCregs of the invention and/or a pharmaceutical composition of the invention comprising human ILCregs of the invention for use in the treatment or prevention of a disease. This may otherwise be referred to for use in therapy. In particular, the invention provides a human ILCreg of the invention, an in vitro population of human ILCregs of the invention and/or a pharmaceutical composition of the invention comprising human ILCregs of the invention for use in the treatment or prevention of an inflammatory disease, such as such as an autoimmune disease, an infection or cancer.

Also provided is the use of the immune cells, the in vitro population and/or the pharmaceutical composition of the invention for the manufacture of a medicament for the treatment or prevention of a disease. Optionally, the disease is cancer or autoimmune disease. In some embodiments the disease is cancer. Further provided is use of immune cells, the in vitro population and/or the pharmaceutical composition of the invention for therapy. Also provided is use of immune cells, the in vitro population and/or the pharmaceutical composition of the invention for the treatment or prevention of cancer. The invention also provides use of immune cells, the in vitro population and/or the pharmaceutical composition of the invention for the treatment or prevention of autoimmune disease.

Also provided is the use of a human ILCreg of the invention, an in vitro population of human ILCregs of the invention and/or a pharmaceutical composition of the invention comprising human ILCregs of the invention for the manufacture of a medicament for the treatment or prevention of a disease. Also provided is use of a human ILCreg of the invention, an in vitro population of human ILCregs of the invention and/or a pharmaceutical composition of the invention comprising human ILCregs of the invention for the treatment or prevention of a disease. In both instances, the disease is preferably an inflammatory disease, such as such as an autoimmune disease, an infection or cancer. Further provided is use of a human ILCreg of the invention, an in vitro population of human ILCregs of the invention and/or a pharmaceutical composition of the invention comprising human ILCregs of the invention for therapy.

Throughout the description and claims of this specification, the words “comprise” and “contain” and variations of the words, for example “comprising” and “comprises”, mean “including but not limited to”, and do not exclude other components, integers or steps. Moreover the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or combination, unless such features are incompatible.

### BRIEF DESCRIPTION OF THE DRAWINGS

One or more embodiments of the invention will now be described, by way of example only, with reference to the accompanying drawings, in which:

FIG. 1: Murine bone marrow derived ILCP yield group 1, group 2, and group 3 ILC in co-culture with epithelial-only SIO. a) Confocal crossection of apical ZO-1 (green) and crypt Paneth cell Lyzozyme 1 expression (magenta) within an SIO (scale bar 50 μm). b) Diagram of co-culture system, with corresponding representative confocal image of SIO (EpCAM-magenta, Nuclei-blue) in co-culture with ILCP (CD45.2-green) represented as a 3D-maximum intensity projection (scale bar 50 μm). c) Schematic of experimental design. d) Representative flow plot of murine bone marrow derived DAPI, Lineage-(HSC cocktail (CD3, CD45R, CD11b, TER-119) Ly-G6, CD5, CD19, and NK1.1), CD127⁺, α4β7⁺, Flt3⁻, PD-1⁺ ILCP and CD25 ILC2P, gated based on PD-1 fluorescence minus one (FMO) controls (overlayed in red). e) Quantification of ILCP precursor count yielded per animal (N=15 animals). f) Representative flow plots of DAPI⁻ EpCAM⁺CD45⁻ epithelial cells and EpCAM-CD45⁺ immune cell populations from 7 day cultures of PD-1⁺ ILCP only, ILCP+SIO, and SIO only cultures, with arrows indicating expression of Lineage markers within the CD45⁺ population. g) Fold change of CD45⁺ Lineage-cells yielded after culture relative to the number of seeded precursors (N=15 animals across 5 experiments). h) Countour plot overlay representing expression of NKp46, RORγt, NKp46, NK1.1, Klrg1, and Sca-1 in Lineage populations derived from (f) in populations pregated as putative group1 (magenta; Live EpCAM⁻ CD45⁺ Lin− RORγt⁻, Klrg1⁻, NK1.1⁺, NKp46⁺), group 2 (green; Live EpCAM⁻ CD45⁺Lin− RORγt⁻, NKp46⁻, Klrg1⁺, Sca-1⁺) NKp46⁺ group 3 (lavender; Live EpCAM-CD45⁺ Lin− Klrg1⁻, RORγt⁺, NKp46⁺), NKp46⁻ group 3 (blue; Live EpCAM⁻ CD45⁺ Lin-Klrg1⁻, RORγt⁺, NKp46⁻), and a putative immature “other” population (grey; Live EpCAM-CD45⁺ Lin− RORγt⁻, Klrg1⁻, NK1.1⁻, NKp46⁻) in cultures derived from ILCP only or ILCP co-culture with SIO. i) Quantification of pooled ILC groups depicted in (h) (ILCP from N=4 animals). Error bars S.E.M, p-values unpaired student t-tests.

FIG. 2: SIO co-cultures support ILCP and functionally mature ILC in parallel. a) Representative flow contour plots of ILC yielded from PD-1+ILCP co-cultures with SIO (color overlays from PD-1⁺+SIO conditions indicating excluded populations) or without SIO (NEG: black=ILCP+SIO magenta=ILCP cultured without SIO). b) Relative frequency of α4β7⁺PD-1⁺CD25⁺c-KIT⁺ of all Lineage-cells c) Relative frequency of RORγt⁻CCR6⁻NK1.1⁻ NKp46⁻Klrg1⁻ST2⁻ (NEG) within the Lineage population, count of NEG, % of CD25⁺, c-KIT⁺, α4β7⁺ and PD-1⁺ within the negative population (frequency of parent, S.D). d) Frequency and count of ILC1 (Live EpCAM⁻ CD45⁺ Lin⁻ RORγt⁻ Klrg1⁻ NK1.1⁺NKp46⁺ Eomes⁻ T-bet⁺) and NK cells (Live EpCAM⁻ CD45⁺ Lin⁻ RORγt⁻ NK1.1⁺ NKp46⁺ T-bet⁺ Eomes⁺) depicted in (e) derived from PD-1⁺ ILCP co-cultures with or without SIO, and compared to putative ILC3 (Live EpCAM⁻ CD45⁺ Lin⁻ RORγt⁺ Klrg1⁻ NK1.1⁻ NKp46^{+/-} Eomes⁻ T-bet^{+/-}) and ILC2 (Live EpCAM⁻ CD45⁺ Lin⁻ RORγt⁻ Klrg1⁺NK1.1⁻ NKp46^{+/-} Eomes⁻ T-bet⁻) (N=7 animals, two experiments f) Frequency of ILC1 and NK cells expressing IFN-γ (FMO overlayed in blue in e) and GranzymeB after 4 h stimulation with PMA/Ionomycin and IL-18 when compared to ILC1 and NK cells isolated from primary small intestine lamina propria, depicted in (g). h) Frequency and count of ILC3 (Live EpCAM⁻ CD45⁺Lin⁻ NK1.1^{+/-} NKp46^{+/-}, Klrg1⁻ ST2⁻, RORγt⁺, CCR6⁻) and CCR6⁺ (Live EpCAM⁻ CD45⁺ Lin⁻, NK1.1^{+/-} NKp46^{+/-}, Klrg1⁻ ST2⁻, RORγt⁺, CCR6⁺) LTi-like cells depicted in (i) derived from PD-1⁺ ILCP co-cultures with or without SIO, and compared to primary ILC3 (N=5 animals, two experiments) j) Frequency of ILC3 and CCR6+LTi-like cells expressing IL-22 (FMO overlayed in blue in ei) and IL-17A (FMO overlayed in magenta in i) after 4 h stimulation with PMA/Ionomycin and IL-23 we when compared to ILC3 and Lti cells isolated from primary small intestine lamina propria, depicted in (k). Error bars S.E.M, p-values unpaired student t-tests.

FIG. 3 is a) a Representative flow plot of IFN-γ expression after 4 h PMA/Ionomycin stimulation in T-bet+ ILC1 (RORγt− NKp46+, NK1.1+, Klrg1−) with corresponding quantification (N=8 mice across at least two experiments). b) Representative flow plots overlaying putative ILC1, NK cells, ILC3, and ILC2 derived from co-culture of PD-1-ILCP with or without SIO with corresponding quantifications. All error bars S.E.M.

FIG. 4 is a) Representative flow plots overlaying putative ILC2, ILC1, ILC3, and Lti-like cells after SIO co-culture with PD-1+ILCP, PD-1-ILCP, and CD25+ ILC2P with corresponding quantification of count and frequency organised by seeded precursor or by resulting ILC populations (N=3 mice). b) Representative flow plot of IL-22 and IL-17A expression in ILCP-derived ROR-γt+ group 3 ILC after 3 h stimulation with PMA/Ionomcyin and IL-23 with corresponding quantification (N=8 mice). All error bars S.E.M . . .

FIG. 5: SIO provide an essentially germ-free model of gut-specific ILC imprinting, maturation, and expansion. a) Representative flow plots of NKp46 expression in CD45⁺Lineage⁻ (CD127⁺) PD-1⁺ ILCP+SIO co-culture derived or primary small intestine lamina propria derived RORγt⁺ ILC3, with the frequency of NKp46⁺ ILC3 (Live EpCAM-Lin CD45⁺ RORγt⁺) additionally quantified for ILCP cultured without SIO or with GF SIO in (b) (N=3-5). c) Pie chart representation of relative group 1 (magenta; Live EpCAM⁻ CD45+Lin⁻ RORγt⁻, ST2⁺Klrg1⁻, NK1.1⁺, NKp46⁺), group 2 (green; Live EpCAM⁻ CD45⁺ Lin− RORγt⁻, NKp46⁻, ST2⁺ Klrg1⁺, Sca-1⁺) NKp46⁺ group 3 (lavender; Live EpCAM⁻ CD45⁺ Lin⁻, ST2⁻ Klrg1⁻, RORγt⁺, NKp46⁺), NKp46⁻ group 3 (blue; ST2⁻, Klrg1⁻, RORγt⁺, NKp46⁻), ILC in live, unstimulated co-cultures derived from SPF-SIO or GF-SIO compared to primary SPF ileum (without LTi-rich Peyer's Patches). d) Diagram illustrating transwell culture strategy. e) Quantification of the relative frequency of group 1, group 2, and group 3 ILC derived from PD-1+ILCP co-cultures with SIO or with SIO separated by a transwell (TW) insert (N=3). f) Count of putative Lineage⁻ RORγt⁻, NKp46⁻, Klrg1⁺, Sca-1⁺ ILC2 and g) Geometric mean fluorescence intensity (GeoMFI) of Klrg1 in Lineage⁻ ILC after co-culture of CD25⁺ ILC2P with SIO, TW-separation, or without SIO (N=4). h) Representative flow plots with respective FMO overlays in cyan and magenta indicating expression of Gata3, IL-25R, IL-13, and IL-15 in ILC2P-derived and small intestine lamina propria derived ILC2 after 4 h stimulation with PMA/ionomycin, quantified in (i-k) (error bars S.D., N=3) I) Gene expression heatmap (magenta=high, cyan=low, white=not detected) of genes of interest derived from bulk RNA-sequencing of EpCAM⁺CD45⁻ intestinal epithelial cells after 7-day co-culture with precursor-derived lymphocytes, without immune cells but with IL-2 and IL-7 supplementation, or in basal SIO media. m) Schematic of metabolite microinjection strategy. n) SIO microinjected with 20 kDa FITC-dextran and 5 mM succinate retain dye within pseudolumen for at least 16 h after injection, indicating tight junctions are retained in presence of metabolite. o) Representative confocal images of SIO microinjected with PBS or with 5 mM succinate in PBS stained for Tuft-cell marker Double cortin like kinase 1 Dclk1 (green, scale bar 50 μm). p) Expression of II25/II17E normalised to housekeeping gene Hprt1 in SIO injected with PBS or Succinate (n=3 wells of SIO in one experiment). q) Frequency of KIrg1⁺ ILC2 after co-culture of ILC2P with SIO injected with PBS or with 5 mM succinate (ILC2P split between conditions from N=4 animals). Error bars S.E.M, p-values unpaired student t-tests.

FIG. 6: Co-culture with gut and lung organoids drive tissue-specific ILC2 imprinting. a) Representative brightfield and confocal image of murine primary distal lung epithelial organoid (LO) with cystic or saccular structures, consisting of enlarged EpCAM⁺ (green) buds (scale bar 25 μm). b) Fold change expansion of Live, EpCAM⁻CD45⁺ ILCP after 7-day co-culture with SIO or LO. c) and d) Pie chart of the frequency of Live, EpCAM⁻CD45⁺ group 1 ILC (magenta, Live, EpCAM⁻, CD45⁺, Lineage⁻, RORγt⁻, NKp46⁺, NK1.1⁺, Gata3⁻), ILC2 (green, gated as Live EpCAM⁻, CD45⁺, Lineage⁻, RORγt⁻, NKp46⁻, NK1.1⁺, Gata3⁺), and ILC3 (blue EpCAM⁻, CD45⁺, Lineage⁻, RORγt⁺, NKp46^{+/-}, NK1.1^{+/-}, Gata3−) derived from 7-day co-culture of of PD-1^{+/-} ILCP with SIO or LO, with corresponding quantification of ILC2 frequency (N=4). e) Representative flow plots of Klrg1, ST2, ICOS, CD25, IL-13, and IL-5 expression in in EpCAM⁻, CD45⁺, NKp46⁻, NK1.1⁻ RORγt⁻ putative ILC2 after co-culture with SIO or with LO (IL-13 and IL-5 FMOs overlayed magenta and blue), with the GeoMFI of IL-5 and IL-13 cytokine expression in ILC2 and IL-22 expression in RORγt⁺ ILC3 quantified in (f) after 4 h unbiased PMA/Ionomycin stimulation (N=4). g) Schematic of gut-to-lung swapping experimental design to assess development versus plasticity of tissue specific ILC2 phenotypes. h) Frequency of Klrg1⁺, ST2⁺, and CD25⁺ ILC2 (relating to figure e) after 14-day co-culture following experimental design layed out in (g) (N=3-5). i) Relative gene expression of Il33 in whole primary small intestine and lung tissue (N=5 animals) as well as in epithelial-only SIO and LO (n=3 wells of organoids) in (j). k) Frequency of Klrg1⁺, ST2⁺, and CD25⁺ ILC2 in SIO-to-LO swapped co-cultures with and without neutralising dose of rmIL-33 blocking antibody (50 ng/ml, N=3). Error bars S.E.M, p-values unpaired student t-tests.

FIG. 7 shows representative flow plots of overlaying putative group 1, group 2, and group 3 ILC after 7 day co-culture with murine lung organoids (LO).

FIG. 8 shows a) Eucledian heatmap (average linkage) of genes of interest expressed in human gut organoids (G;N=2) and lung organoids (L; N=2), cultured for 24 h or 72 h, not infected (NI) or infected (I) with Cryptopsporidium, representative of N=3 experiments. Values represent [log (X+1,2)] fold change (FC) relative to housekeeping genes (HKGs) ACTB, GAPDH, and HPRT1 (magenta: greater than HKG, cyan: lower than HKG). Black box highlights significantly differential expression of gene of interest IL33 (Two-stage linear step-up of Benjamini, Krieger, and Yekultieli, with Q=1%, p<0.000001; q<0.000001). (Accession: GSE112991) of human primary small intestine and lung epithelial organoids, sequenced after 24 h or 72 h culture with (infected—I) or without (not infected—NI) microinjected Cryptosporidium infection (Heo and Dutta et al., 2018). Ingenuity Pathway Analysis (IPA) was used to annotate these datasets, isolating basally presented or secreted ligands, as well as quality control genes (e.g. CDX2 in intestinal epithelium). b) Volcano plot of data represented in (a), highlighting genes with statistically significant differential expression. c) Frequency of Klrg1, CD25, and ST positive cells among putative ILC2 derived from 7 day SIO co-culture after FACS isolation from co-cultures and reseeding in Matrigel with or without SIO, and without SIO but with rmIL-33 supplementation for an additional 7 days (N=2 mice). d) Frequency of Klrg1, CD25, and ST positive cells among putative ILC2 derived from 7 day LO co-culture after FACS isolation from co-cultures and reseeding in Matrigel with LO or swapped to SIO culture for an additional 7 days. (N=3). Error bars S.E.M., unpaired student t-tests.

FIG. 9: HIO promote proliferation and maturation of systemic human ILCP. a) Gating strategy for healthy PBMC-derived ILCP, pregated on live, single, CD45⁺ cells, with appropriate FMOs overlayed in blue and magenta. b) Schematic of HIO ILCP co-cultures, indicating presence of mesenchymal cells and CD45⁺ ILCP c) Representative flow cytometry plot overlays of EpCAM⁺ intestinal epithelial cells (E, green), double negative mesenchymal cells (M, magenta), and CD45⁺ ILC (blue) after 14 day co-cultures, highlighting Matrigel debris in grey, and quantified in (d) as count of EpCAM-CD45⁺ Lineage; ILC and (e) fold change expansion of ILC after 14 day co-culture relative to the number of ILCP seeded on day 1 (N=3-15 across ~7experiments). f) Count of Live, EpCAM⁻, CD45⁺, Lineage⁻, RORγt⁺ ILC after 14 day co-culture with mesenchyme depleted HIO or epithelial depleted mesenchyme/fibroblasts expressing markers CCR6, NKp44, and/or T-bet (N=3). g) Pie charts of relative group 1 (Live, CD45+ Lin⁻ CRTH2⁻ c-kit, and [CD127⁺, CD161⁺ in primary gut ILC only] and [EpCAM⁻, RORγt⁻, GATA3⁻ in MD-HIO and ED-HIO only) group 2 (Live, CD45⁺ Lin⁻ CRTH2⁺ c-kit^{+/-}, and [CD127⁺, CD161⁺ in primary gut ILC only] and [EpCAM⁻, RORγt⁻, GATA3⁺ in MD-HIO and ED-HIO only), group 3 (Live, CD45⁺ Lin⁻ CRTH2⁻ c-kit⁺ NKp44^{+/-}, and [CD127⁺, CD161⁺ in primary gut ILC only] and [EpCAM⁻, RORγt⁺, GATA3⁻ in MD-HIO and ED-HIO only) and other Lineage⁻ ILC (e.g. undifferentiated precursors, (Live, CD45⁺Lin⁻ CRTH2⁻ c-kit, and [CD127⁺, CD161⁺ in primary gut ILC only] and [EpCAM⁻, RORγt⁻, GATA3⁻ in MD-HIO and ED-HIO only) in unstimulated, primary human intestine (N=13, adapted from Kraemer et al., 2017), MD-HIO (N=13), and ED-FB (N=7). h) Representative image of CD45+ILCP co-cultured with mesenchyme-depleted, E-cadherin⁺ HIO (scale bar 25 μm). Error bars S.E.M, p-values unpaired student t-tests.

FIG. 10. a) Representative confocal image of a human intestinal organoid (HIO, apical actin ring magenta, hindgut expression of transcription factor CDX2 white, Nuclei stained with DAPI, cyan) in co-culture with ILCP (CD45, yellow arrows). Mesenchyme surrounds complete epithelial-mesenchymal HIO structures Comp. HIO) as CD45-CDX2-nuclei (blue arrows). Scale bar 50 μm. b) Count of seeded ILCP (day 1), CD45 immune cells after 14 day co-culture with or without complete epithelial-mesenchymal HIO. c) Representative flow plot of GATA3 and IL-13 expression in CD45⁺ Lineage ROR-γt⁻ CRTh2⁺ ILC2, stimulated with PMA/Ionomcyin for 4 h, with corresponding quantification of expression in day 1 ILCP pre-co-culture and 14 day co-culture with complete HIO (ILCP from N=3 donors). d) Overlays of target gene expression in putative group 1 (magenta⁻ Live, EpCAM⁻ CD45⁺ LIN⁻ RORγt^{low} T-bet⁺), and group 3 (Live, EpCAM⁻ CD45⁺ LIN⁻, ROR-γt⁺) cells expressing CCR6 (orange), NKp44 (dark blue), and no NKp44 (light blue) after 4 h stimulation with PMA/Ionomycin after 14 day co-culture with mesenchyme depleted HIO (MD-HIO). e) Overlay of putative group 3, T-bet⁺ and Eomes⁺≥group 1 ILC, showing expression of IFN-γ in these PMA/Ionomycin stimulated co-cultures with complete HIO with or without IL-18 stimulation, with corresponding quantifications. f) Representative flow plots CD161 and RORγt expression in Live EpCAM⁻ CD45⁺ LIN⁻ HIO-derived ILC, with corresponding quantification of CD161⁺ RORγt⁺ cells and g) the frequency of NKp44⁺ ILC within the CD161+ RORγt+ population. h) Representative flow plots of IL-22 and IL-17A expression with corresponding quantification in complete HIO-derived ILCP after 4 h stimulation with PMA/Ionomycin with or without additional IL-23 stimulation. Error bars S.E.M., unpaired student t-tests, ILCP from N=3 donors.

FIG. 11: Human epithelial cells, not mesenchymal cells, drive proliferation and maturation of functional human ILC. a) Frequency of Live, EpCAM⁻, CD45⁺, Lineage⁻, RORγt⁺ ILC after 14 day co-culture expressing markers CCR6, NKp44, and/or T-bet. b) Frequency of IL-22⁺ and IL-17A⁺ MD-HIO and ED-FB derived ILC after 4 h stimulation with PMA/Ionomycin. c) Representative flow plots corresponding to (b) (FMOs overlayed in blue and magenta). d) Frequency of CD45⁺ LIN⁻ RORγt⁻ expressing T-bet and/or Eomes e) Representative flow plots of CD56 and IFN-γ expression in T-bet⁺ and Eomes⁺ populations after 4 h stimulation with PMA/Ionomycin (IFN-γ FMO overlayed magenta), with corresponding quantification. f) Total count of putative ILC2 after 14 day co-culture. g) Expression of IL-5 and IL-13 in putative ILC2 after 4 h stimulation with PMA/Ionomycin (FMOs overlayed magenta and blue). With corresponding quantification. Error bars S.E.M, p-values unpaired student t-tests; ILCP from N=3 donors.

FIG. 12. Representative flow plots of group 1 associated genes visualised by overlaying ROR-γt⁻ T-bet⁺ (red), Eomes⁺ (purple), or “other” T-bet-(grey) after 14 day co-culture with MD-HIO or ED-FB after 4 h stimulation with PMA/Ionomycin, with relevant quantification of geometric mean fluorescence intensities. Error bars S.E.M., unpaired student t-tests, ILCP from N=3 donors.

FIG. 13. a) Relative frequency among CD45⁺ Lineage⁻ of putative ILC2 (gating strategy in (b)), after 14 day co-culture with MD-HIO or ED-FB. b) Representative flow plots overlaying putative ILC2 (green) and other Klrg1⁻ non-ILC2 (magenta) showing expression of group 2 associated genes after 4 h stimulation with PMA/Ionomycin (ILCP from N=3 donors). c) Representative flow plots of putative ILC2 and IL-5/IL-13 expression after 4 h stimulation with PMA-Ionomycin after ILCP co-culture with MD-HIO, MD-HIO separated from ILCP by a transwell separator, or ILCP cultured in Matrigel without organoids (N=1 donor, n=2 technical replicates). d) Count of EpCAM⁻CD45⁺ immune cells and EpCAM−CD45− fibroblasts/mesenchyme in co-cultures derived from 14-day MD-HIO co-cultures, which were then FACS purified and reseeded either with fresh MD-HIO or with ED-FB. e) Frequency of IL-13⁺ and IL-5⁺ ILC within putative ILC2 pre-matured with MD-HIO and reseeded with MD-HIO or ED-FB for an additional 7 days. ILCP from N=3 donors. All error bars S.E.M., unpaired student t-tests.

FIG. 14. Transfer of gut-matured ILC to HLO recapitulate tissue-specific human ILC2 phenotypes. a) Representative image of HIO and human lung organoids (HLO) showing E-cadherin⁺ epithelium (magenta), CD45⁺ ILC (yellow), and nuclei (Hoechst, cyan) after 14-day co-culture (scale bars 50 μm). b) Count of EpCAM⁻, CD45⁺, LIN⁻ ILC after 14-day co-culture with MD-HIO or MD-HLO, with corresponding count of EpCAM⁻ CD45⁻ fibroblasts/mesenchyme. c) Representative flow plots of RORγt, CCR6, IL-22, IL-17A, IFN-γ, and NKp44 after 14 day co-culture with MD-HIO or MD-HLO after 4 h PMA/Ionomycin stimulation (pregated population indicated in grey, representative of N=3). d) IL-5 and IL-13 expression in putative GATA3⁺ ILC2 after 14 day co-culture and 4 h PMA/Ionomcyin stimulation (FMOs overlayed magenta and cyan) with corresponding quantification. e) Expression of CD25 and ST2 in putative GATA3⁺ ILC2 after 14 day co-culture with HIO or HLO with corresponding quantification. f) Quantification of the relative frequency of CD25⁺ and ST2⁺ putative ILC2 and (g) MFI of ST2 in the same population with corresponding histogram overlay of ST2-PE after 14 day co-culture with HIO or HLO, followed by a reseeding from HIO back to HIO, a swap from HIO to HLO, or a swap from HIO to HLO with 50 ng/ml hIL-33 neutralising antibody. All experiments performed with ILCP from N=3 donors, unpaired two-tailed student t-tests, error bars S.E.M.

FIG. 15. Expression of HLA-DR on ILC3 generated from 13 days co-culture of ILC precursors (ILCP) on mesenchymal depleted human intestinal organoids (MD-HIO). Showing percentage of HLA-DR+ from the ILC3 population pre-gated on Live, CD45+, Lin− (CD3− CD20− CD14− CD19−), ckit⁺ ILC3. Red: ILC3, blue: fluorescence minus one (FMO) control.

FIG. 16. Antigen processing by ILC via MHC-II shown by hydrolysis of DQ-BSA each leads to fluorescent signal. a) Expression of Dye Quenched-Bovine Serum Albumin (DQ-BSA) fluorescence in expanded ILCs followed by a 4 hour incubation with DQ-BSA at 37° C., on Ice or without DQ-BSA. ILCs were expanded from ILC precursors derived from healthy adult PBMCs. (CD45+, Lin− then ILC1s are cKit− CRTH2−, CD56− and CD161+, ILC2s are CRTH2+ and ILC3s are ckit+, CRTH2−). b) Expression of HLA-DR in expanded ILC populations following 4 hour incubation with Dye Quenched-Bovine Serum Albumin (CD45+, Lin− then ILC1s are cKit− CRTH2−, CD56− and CD161+, ILC2s are CRTH2+ and ILC3s are cKit+, CRTH2−). c) Expression of HLA-DR in expanded ILC populations following 4 hour incubation with Dye Quenched-Bovine Serum Albumin where grey dots are DQ-BSA-ILCs (CD45+, Lin— then ILC1s are cKit− CRTH2−, CD56− and CD161+, ILC2s are CRTH2+ and ILC3s are cKit+, CRTH2−).

FIG. 17. Single cell RNAseq (scRNAseq) on the organoid-generated ILCs. A) UMAP visualisation of all cells which passed quality controls (n=7,446) color-coded based on annotated ILC subsets. B) Dot plot showing the relative enrichment of the selected and previously described human ILC subset-specific genes used to annotate the clusters. Whilst the gene expression of ILCregs has yet to be characterised within the human intestine, key genes used to differentiate these cells from the other ILC subsets in mice (Wang et al., 2017) were found to be highly expressed in this cluster, including IL10, SOX4 and ID3, alongside other genes characteristic of Tregs (e.g., FOXP3, CTLA4 and IL2RA). C) UMAP visualisation of the enrichment scores of a human ILCP gene signature (Liu et al., 2021). Despite these cells not forming their own distinct cluster, their clear clustering observed here within the ILC3 population indicates that a small proportion of ILCPs were captured and sequenced. D) UMAP visualisation of the density of TBX21 and EOMES expression. Density mapping was employed here due to the weak detected expression of these two transcription factors. At the resolution used here, ILC1 and NK cells do not form distinct clusters; however, the patterning of EOMES expression suggests that both cell types were successfully generated within this co-culture. E-F) Density plot and UMAP visualisations of the enrichment scores of gene modules associated with human tissue specific ILC populations (Mazzurana et al., 2021) in ILC3s. The three modules examined here correspond to a circulatory ILC module (mod11), a colonic ILC module (mod3) and a lung ILC module (mod34). Only ILC3s were examined as Mazzurana et al., did not identify any distinct clusters of ILC1s or ILC2s in the colon. Despite ILC3s exhibiting greatest enrichment of a circulatory phenotype, this was not uniform across the ILC3 cluster (F) with the cluster dividing into those cells showing a greater enrichment for a circulatory ILC signature, which includes the putative ILCP cells, and those displaying a more colonic (mod3) enrichment. Notably, these ILC3s exhibited the weakest enrichment for the lung ILC gene module. This indicates that within the intestinal co-cultures, ILC3s may mature and differentiate towards a more intestinal tissue-specific phenotype.

FIG. 18. NK cells generated by co-culture of ILC precursors (ILCP) with intestinal organoids are cytotoxic. Percentage (%) of dead CFSE stained K562 target cells. CFSE cells (target cells, T) were labelled carboxyfluorescein succinimidyl ester (CFSE) prior to co-culture with CD56+NK cells (effector cells, E) expanded from ILCP in human intestinal organoid co-cultures. They were co-cultured at different effector-to-target (E:T) ratios from 5:1, 2.5:1 to 1.25:1 with IL-2 supplementation (20 ng/ml). Three control samples were prepared, target cells alone, effector cells and a positive control for cell dead (targets cells treated with Tween 20). To determine cellular viability, all experimental groups were stained with LIVE/DEAD fixable UV blue stain.

FIG. 19. Generation of ILCs in human small intestine biopsy derived organoids. Generation of differentiated ILCs (including ILC1, ILC2, ILC3 and NK cells) from ILC precursors (ILCP) after 15 days in culture with human small intestine biopsy derived organoids in which 50% Intesticult™ (commercially available from StemCell Technologies) and 50% homemade organoid medium were used. A. Cell counts at the start and end of the culture. B. Visual representation (brightfield microscope) of the expanded ILCs in contact with human small intestinal organoids (dark areas).

### DETAILED DESCRIPTION

### Materials and Methods

### Animals

All animals were culled by cervical dislocation according to standard ethical procedure, conducted by trained individuals. Slicing of the femoral artery or decapitation was conducted (as appropriate to the protocol in hand) as confirmatory assessments of death, prior to organ and tissue harvest. Animals were housed under specific-pathogen-free conditions (unless stated otherwise) at accredited Charles River and King's College London animal units in accordance with the UK Animals (Scientific Procedures) Act 1986 (UK Home Office Project License (PPL: 70/7869 to September 2018; P9720273E from September 2018).

### Murine Organoids Isolation

Murine epithelial small intestines (ileum) were isolated from 6-8 week female C57BL/6 mice following established protocols (Sato et al., 2011, herein incorporated by reference). Small intestinal epithelial organoids (SIOs) were cultured in 3D Matrigel bubbles and passaged using a bent pipette tip to disrupt the crypts every 5-7 days. SIO were cultured in basal media supplemented with R-Spondin1 (50 μl supernatant/ml from an R-spondin producing cell line or 1 mg/ml), Noggin (50 μl supernatant/ml or 100 μg/ml), and rm-EGF 50 μg/ml).

Murine lung organoids were isolated from distal lung tips following established protocols (McQualter et al., 2010, herein incorporated by reference). Tissue was cut into small 5-20 mm² pieces, rinsed in PBS, then digested using 1.5 mg/ml Dispase II, 0.5 mg/ml Collagenase, and 10 μg/ml DNAse in 10 ml PBS with 2% FCS for 1 h at 37° C. on a shaker set to 100-250 RPM, with a 15 second vortex every 20 min. Samples were then allowed to settle, and a cloudy fraction enriched for fibroblasts and immune cells was discarded. The remaining larger chunks were then incubated in EDTA and HEPES for another hour, before being resuspended in Matrigel, and cultured in basal media with R-Spondin, Noggin, EGF, FGF10 (500 ng/ml), 5 μM CHIR, and 1 μM RhoK-inhibitor for 4 days. Once luminal structures formed, RhoK-inhibitor was withdrawn, and cultures were expanded for a minimum of three weeks to enrich for alveolar basal stem cells. These heterogeneous organoid cultures were then FACS purified to isolate Live, CD45−, EpCAM^{high} epithelial cells, which were expanded as epithelial only structures for 4 weeks in media containing expansion media with Rho-K inhibitor.

### Human Organoid Differentiations

KUTE-4 (Leha et al., 2016) and FS13B human iPSCs previously generated using established protocols (Kilpinen et al., 2017; Yusa et al., 2011, both incorporated by reference). Human iPSCs were maintained on Vitronectin (StemCell Technologies) in E8 media and passaged as disrupted clusters every 4-6 days using Versene (GIBCO).

HIO were derived following previously established protocols (McCracken et al., 2011, incorporated by reference), with substitution of CHIR99021 for recombinant Wnt3a. HIO were further matured through addition of 20 ng/ml IL-2 to the expansion media. HIO were passaged and reseeded in Matrigel as whole structures every 10-14 days. Organoids were matured for 4-8 weeks before use in experiments.

### Murine ChILP and ILC Isolation

ILCP were isolated from 6-8 week C57BL/6 female murine femur and tibia bone marrow (BM) following established protocols (Gronke et al., 2017, herein incorporated by reference). Soft tissue was physically removed from bones, which were subsequently sterilised in 70% ethanol for 2 min and rinsed in ice cold PBS. The ends of the bones were cut off with dissecting scissors, and the middle section was then then flushed with PBS using a 27-gage needle. Marrow was triturated, transferred to a 50 ml falcon tube, and centrifuged at 1500 RPM/500 G for 3 min. After removal after supernatant, red blood cells were depleted using 2 ml standard ACK lysis buffer for 2 min at room temperature. The reaction was quenched with 30 ml PBS and centrifuged at 500×G for 3 min. The remaining pellet was resuspended in PBS supplemented with 2% FCS, 0.1 M EDTA, and 1 mM HEPES (FACS buffer), and strained into a flow tube through a 40 μm sterile mesh. Fc receptors were blocked using anti CD16/CD32 (2.4G2) for 10 minute at 4° C. 5 million cells in 100 μl FACS buffer were stained with primary conjugated antibodies in the dark at 4° C. for 30 min, rinsed in FACS buffer, centrifuged, and resuspended in 200-300 μl FACS buffer with 0.1 μg/ml DAPI (4′,6-diamidino-2-phenylindole; Sigma) for dead cell exclusion. Unstained cells and UltraComp beads (eBiosciences) were used for unstained and single colour controls to calculate compensation. Fluorescence minus one (FMO) were used for CD127 (IL 7Rα) and Lineage+DAPI ChiLP were isolated by Fluorescence-activated cell sorting (FACS) on an ARIA-III (BD Biosciences) using DIVA software, with assistance from the Guy's King's St Thomas (GSST) Biomedical Research Council (BRC) flow core staff.

### Human ILCP Isolation

Systemic ILCP were isolated from leukocyte cones (NHS-BT) following established protocols (Lim et al., 2017a, herein incorporated by reference). Briefly, lymphocytes were purified from cones using FICOLL density gradient separation. A small aliquot from each cone was phenotyped using the hILCP panel to assess ILCP frequency and titre antibodies, and the remaining cone was distributed between 20-30 cryo-preserved vials of approximate equal hILCP frequency (10% DMSO in foetal calf serum, added dropwise and stored in liquid nitrogen after being frozen in a Mr Frosty Isopranol container), allowing for experiments to be performed from the same three donors to reduce variability and ensure that sufficient Lineage antibody was added to exclude non ILC subtypes. On day-1 of experiments, cryovials were thawed and rested in media containing 10% FCS and Fc block for 1 h, rinsed with PBS for fixable Live/Dead staining, stained and sorted on a FACSARIA-III (BD biosciences).

ILCP Co-Cultures with Organoids

On day 1, murine SIO/LO were mechanically disrupted to a whole crypt state, and HIO/HLO were digested with 0.1 mg/ml Collagenase for 15 min at 37° C., then mechanically disrupted until a cloudy mesenchyme-enriched fraction appeared. Epithelial structures were allowed to settle to the base of a 15 ml falcon tube, and the mesenchyme-rich fraction was removed, repeating this step 3-5 times or until the epithelial-enriched fraction became clear. Approximately 50-100 murine and 25-50 human organoid structures were transferred to 1.5 ml eppendorfs, then approximately 500-1000 ILCP were added to these tubes, and centrifuged at 300 G for 5 min. After carefully removing the supernatant, the resulting cell mix was rigorously resuspended in 30 μl ice cold Matrigel, avoiding bubble formation, and the combined cell mixes were pipetting in the centre of a pre-warmed tissue-culture treated well, and allowed to solidify for at least 15 min at 37° C. Pre-warmed basal media supplemented with R-Spondin, Noggin, EGF, and with 50 mM 2-mercaptoethanol (R&D), 20 ng/ml rhIL-2 (Sigma), and 20 ng/ml rmIL-7 (R&D) was then carefully added to each well. This medium may otherwise be referred to herein as Medium A.

Half media changes (50% media out, replenished with 60% of the remaining volume) were performed every 1-3 days, allowing for conditioned media to remain in the wells while supplementing with fresh 2× growth factors to ensure viability of organoids and ILC without disrupting ILC-epithelial interactions. No small molecules, FGF7, or FGF10 were supplemented to lung organoid cultures to maintain consistency between conditions.

For transwell experiments, permeable inserts separated the organoid fraction (top) and the ILCP fraction (bottom), with both resuspended in 25 μl Matrigel (Falcon 24 well (Corning), 1.6×10⁶ pores per cm²).

In inter-organ swapping experiments, ILCP+organoid co-cultures were dissociated with TryPLE on day 7, and live EpCAM−, CD45+, Lineage− whole populations were isolated from half of the culture by FACS, with the other half being used for flow cytometry analysis. This prevented epitopes from being blocked for secondary analysis on day 14. The organoid-matured ILC populations yielded by FACS were then re-seeded with the same or the opposite organoid cultures in fresh Matrigel, and the protocol was restarted as on day 1.

For regular analysis, day 7 or day 14 (swapped) co-cultures were either fixed in 4% PFA for immunocytochemistry or rinsed with PBS and dissociated with TrypLE (Gibco) and DNAse (due to potential dead epithelial cells) for 20 min to obtain single-cell suspension to be analysed by flow, or to FACS purify individual populations for RT-qPCR. Any suspension containing single epithelial cells were maintained in 2% FCS with 0.1 mM EDTA and 1 mM HEPES to reduce cell clumping.

### Co-Culture Analysis

### Flow Cytometry

Flow cytometry data was acquired on a Fortessa II (BD Biosciences) using DIVA software and analysed using FlowJo 10.4.1.

### RT-qPCR

RNA was extracted using the RNAeasy micro kit (Qiagen), with 10 μl/ml β-ME supplemented to the RLT lysis buffer to mitigate degradation in RNAse-rich epithelial tissues. cDNA reverse transcription was performed following manufacturer's protocols with the RevertAid synthesis kit (ThermoFischer), using 0.5 μl random primer and 0.5 μl Oligo dTTT primer per 10 μl reaction. RTqPCR were with primers ordered from Invitrogen with SYBR (Applied Bioscences) or with TAQ-FAM probes (ThermoFisher) with TAQ enzyme (Applied Biosciences) and run on a BioRad Real Time CFX384 Touch with CFX Maestro software, and resulting data were processed and normalised in Microsoft excel.

### Confocal and Live Imaging

Live imaging was performed overnight on co-cultures 1 day post-seeding for Cell Trace FarRed experiments where ILCP were labelled prior to co-culture, or on day 4 for ILCP cultures with Rorc^{eGFP} animals. Images were taken using a NIKON A1R inverted confocal microscope with incubation capabilities and using phenol free media. Co-cultures were then fixed in 4% PFA for 5-15 min at room temperature within 3D Matrigel bubbles to retain relative ILC-organoid localisation. Samples were either stained as whole organoids, or were cryo-preserved in 30% glucose, then embedded in OCT overnight at 4° C. to desiccate the Matrigel, then frozen and cryosectioned. Samples were permeabilised using 0.05% Triton-X, stained in primary antibody overnight at 4° C., and stained in secondary antibody and Hoechst for 1 h at room temperature (RT). Images were acquired on a Leica SP8 confocal microscope using LAX software, and resulting images were processed using FIJI (ImageJ).

### RNA-Sequencing of Murine SIO

Lymphocyte precursors (Lin-Cd127+α4β7-Ftl3+) were harvested from the BM and sorted as described above and cultured with SIO in the presence of 50 mM β-mercaptoethanol (R&D), 20 ng/ml rhIL-2 (Sigma), 20 ng/ml rmIL-7 (R&D) and 20 ng/ml Flt3-ligand (R&D) for 7-days. EpCAM⁺Cd45⁻ were sorted into lysis buffer and RNA was extracted as described above. The library was prepared using SMARTer Stranded Total RNA Seq kit-pico input mammalian and sequenced using HiSeq 2500 at the King's College London Genomics Centre, where basic alignment and quality control were also performed. Normalised count values were represented as (logX+1,2) in Excel 16.16.20, and represented as a heatmap in GraphPad Prism 8.2.1.

### Quantification and Statistical Analysis

Data was analysed using Microsoft Office 365 Excel 16.16.20 and GraphPad Prism 8.2.1. Meta-analysis of public deposited RNA-sequencing was performed by normalising raw FPKM values to the Geometric mean of housekeeping genes Actb/ACTB, Hprt1/HPRT1, and Gapdh/GAPDH, and the (logX+1, 2) of these values was analysed with multiple row t-tests. Log-q values were visualised as Volcano plots in GraphPad Prism 8, heatmaps were produced using heatmapper.ca/expression/, applying clustering to rows and columns (applying clustering dendrograms to columns) using centroid linkage and Eucledian distance measurement.

### Reagents and Models Used

TABLE 1
Experimental Model and Subject Details

| MODEL/CELL | |
|---|---|
| LINE | SOURCE |
| KUTE-4, BOBC, | Feeder-free hiPSC derived from skin tissue via |
| and FS13B | CtyoTune 2 in 2015 as part of the HipSci |
| human induced | consortium phenotyping project (Kilpinen, |
| pluripotent stem | Goncalves, Lega et al. Nature (2017). The cell |
| cell lines | lines are banked at ECACC and have catalogue |
| | number 77650426, disease status of the female |
| | anonymous donors was normal, and all details |
| | and pluripotency scores can be found at |
| | http://www.hipsci.org/lines/#/lines/HPSI0714i- |
| | kute_4. The hiPSC are titled HPSI0714i-Kute_4. |
| | The Wellcome Trust Sanger Institute and the |
| | Wellcome Trust and MRC Cambridge Stem Cell |
| | Institute provided human iPS cell lines. |
| | Mycoplasma testing was performed by the King's |
| | College London Centre for Stem Cells and |
| | Regenerative Medicine on a monthly basis. |
| PBMC Leukocyte | NHS-BT |
| cones | |
| C75BI/6J | 6-12 weeks females were used. |
| Germ Free | 6-12 weeks females were used. Colony was |
| C75BI/6J | maintained by MAC in IVCs inside isolators under |
| | axenic conditions. |
| CD45.1, | 6-8 week old females were purchased from |
| C75BI/6J | Charles River. |

TABLE 2
Antibodies

| REAGENT | SOURCE | IDENTIFIER | CLONE |
|---|---|---|---|
| Mouse Lineage cocktail- | eBioscience | 88-7772-72 | 17A2, RA3- |
| PacBlue | | | 6B2, M1/70 |
| Mouse CD3-PacBlue | eBioscience | 48-0031-82 | 145-2C11 |
| Mouse CD19-PacBlue | eBioscience | 48-0193-82 | eBio1D3 |
| Mouse Ly6g-PacBlue | eBioscience | 48-5931-82 | RB6-8C5 |
| Mouse CD5-PacBlue | eBioscience | 48-0051-82 | 53-7.3 |
| Mouse NK1.1-PacBlue, PE, | eBioscience | 48-5941-82 | PK136 |
| | | 9(e450), 12-5941- | |
| | | 83(PE), | |
| Mouse CD127-APC | eBioscience | 17-1271-82 | A7R34 |
| Mouse a4b7-PE | eBioscience | 12-5941-83 | DATK32 |
| Mouse Flt3-PerCP Cy.5. | eBioscience | 46-1351-82 | A2F10 |
| Mouse NK1.1-bv605 | BioLegend | 108753 (bv605) | PK136 |
| Mouse NKp46-bv605 | BioLegend | 137619 | 29A1.4 |
| Mouse Klrg1-PerCP Cy5.5 | eBioscience | 46-5893-82 | 2F1 |
| Mouse NKp46-PE Cy7 | eBioscience | 25-3351-82 | 29A1.4 |
| Mouse CD45-bv510 | BioLegend | 103137 | 30-F11 |
| Mouse Sca-1 bv711 | BioLegend | 108131 | D7 |
| Mouse Sca1-Alexa700 | eBioscience | 56-0451-82 | 30-F11 |
| Mouse EpCAM-APC Cy7 | BioLegend | 118217 | G8.8 |
| Mouse CD25-APC e780 | eBioscience | 47-0251-82 | PC61.5 |
| Tbet-BV711 | BioLegend | 644819 | 4B10 |
| Mouse IL-22-PE | eBioscience | 12-7221-82 | 1H8PWSR |
| Mouse IFN-γ-APC | eBioscience | 17-7311-81 | XMG1.2 |
| Mouse IL-5-APC | BD biosciences | 562048 | TRFK5 |
| Mouse IL-13-PE Cy7 | eBioscience | 25-7133-80 | eBio13A |
| Mouse IL17a-PE/Dazzle | BioLegend | 506937 | TC11- |
| | | | 18H10.1 |
| Mouse IL17a-APC | Invitrogen | 17-7177-81 | eBio17B7 |
| Mouse Gata3-AF488 | BD Pharmingen | 560163 | L50-823 |
| Mouse St2-PerCPe710 | eBioscience | 46-9335-82 | RMST2-2 |
| Mouse RORγt-BV785 | BD biosciences | 564723 | Q31-378 |
| Mouse ICOS-APC | eBioscience | 17-9949-82 | C398.4A |
| Mouse CD25-bv786 | eBioscience | 14-0251-86 | PC61.5 |
| Human CD45-e450, | Invitrogen | 48-0459-42 | HI30 |
| Alexa700 | | | |
| Human EpCAM-FITC | BioLegend | 324203 | 9C4 |
| Human LIN cocktail 3-FITC, | BD biosciences | 643510 | CD3 SK7, |
| PacBlue | | | CD20 L27, |
| | | | CD19 |
| | | | SJ25C1, |
| | | | CD14 MφP9 |
| Human CD4-APC | eBioscience | 17-0048-42 | OKT4 |
| Human KLRG1-APC | BioLegend | 138411 | 2F1-AG |
| Human CD335 NKp46-APC | BioLegend | 331917 | 9E2 |
| Human TCR α/β- | BioLegend | 306706 | IP26 |
| FITC | | | |
| Human TCR γ/δ- | BioLegend | 331208 | B1 |
| FITC | | | |
| Human CD127-PE Cy7 | eBioscience | 25-1278-42 | eBioRDR5 |
| Human CD56-Alexa 700 | BioLegend | 318316 | HCD56 |
| Human c-KIT/CD117 bv605 | BioLegend | 313218 | 104D2 |
| Human CRTh2-PE | Miltenyi Biotec | 130-113-600 | BM16 |
| Human CRTh2-BV711 | BioLegend | 350124 | BM16 |
| Human CRTh2-BV421 | BioLegend | 350112 | BM16 |
| Human CD161 APC, Alexa700 | BioLegend | 302012 | V NK80 |
| Human ST2-APC | R&D Sys | FAB5231A | N/A |
| Human ST2-PE | R&D Sys | FAB5231P | N/A |
| Human NKp44-PE Cy7 | BioLegend | 325116 | P44-8 |
| Human NKp44-PerCP Cy5.5 | BioLegend | 325114 | P44-8 |
| Tbet-PE-Cy7 | BioLegend | 644824 | 4B10 |
| Human RORγt-APC | eBioscience | 17-6988-82 | AFKJS-9 |
| Human RORγt-PE | BDBiosciences | 563081 | Q21-559 |
| Human GATA3-APC Cy7 | Santa Cruz | sc-268, APCC7 | HG3-31 |
| Human IL22-PerCP Cy5.5 | BioLegend | 366709 | 2G12A41 |
| Human IL17A-PE-Dazzle | BioLegend | 512335 | BL168 |
| Human IL17A-e450, BV786 | BD Horizon/ | 560610 (e450), | N49-653 |
| | bioscience | 563745 (BV786) | |
| Human IFNg-APCe780 | Invitrogen | 47-7319-41 | 4S.B3 |
| Human IL-5-APC | BioLegend | 504305 | TRFK5 |
| Human IL-13-FITC | eBioscience | 11-7139-41 | PVM13-1 |
| Human IL-13-bv711 | BDBiosciences | 564288 | JES10-5A2 |
| Human CD25-PerCP-Cy 5.5 | BDBiosciences | 560503 | M-A251 |
| Human Klrg1-APC | eBioscience | 25-5893-80 | 2F |
| Human CCR6-APC | BioLegend | 353416 | G034E3 |
| Human CCR6-BV605 | BioLegend | 353419 | G034E3 |
| Human NKp46 APC | BioLegend | 331918 | 9E2 |
| BLOCKING ANTIBODIES | | | |
| FcR CD16/32 blocking mouse | BioCell | BE0307 | 2.4G2 |
| FcR blocking human | Miltenyi Biotec | 130-059-901 | N/A |
| Anti-rhIL33 (neutralising, | R&D | AF3625 | Ser112- |
| ICC, goat polyclonal) | | | Thr270 |
| Anti-rmIL33 (neutralising, | R&D | AF3626 | Ser109- |
| ICC, goat polyclonal) | | | Ile266 |
| ICC ANTIBODIES | | | |
| E-Cadherin-human (rat) | eBioscience | 51-3249-82 | DECMA-1 |
| CDX2-human (rabbit) | abcam | Ab76541 | |
| EpCAM-mouse (rabbit) | abcam | Ab71916 | |
| Purified anti-human CD45- | BioLegend | 304001 | HI30 |
| (mouse) | | | |
| ZO-1 | Abcam | Ab96587 | Aa 1-266 |
| Dclk1 | Abcam | Ab31704 | AL: 82.2 KDa |
| | | | and BL: 47.6 |
| | | | KDa |
| CD44 | eBioscience | 14-0551-82 | IM7 |

TABLE 3
Cell culture reagents

| | | |
|---|---|---|
| EGF | R&D | 236-EG-01M |
| rhIL-2 (CF) | BioLegend | 589104 |
| rmIL-7 | BioLegend | 577806 |
| rmIL-33 | BioLegend | 580502 |
| FGF10 | PeproTech | 100-26-50ug |
| FGF7 | R&D | 251-KG-010/CF |
| rmFlt3 ligand | R&D | 427_FL |
| IL-22 | PeproTech | 200-22-2uG |
| IL-18 | BioLegend | 592102 |
| rhIL-33 | BioLegend | 581802 |
| Advanced DMEM/F12 | Gibco | 12634-010 |
| Glutamax | Gibco | 1932118 |
| Antibiotic-Antimycotic | Gibco | 15240096 |
| HEPES 1M | Gibco | 2259252 |
| 2-Mercaptoethanol 50 mM | Gibco | 31350-010 |
| PBS | Gibco | 10010-015 |
| PFA 16% | Pierce | 28906 |
| heat inactivated qualified | Gibco | 26140087 |
| FBS/FCS | | |
| N-Acetylcysteine | Sigma-Aldrich | A9165 |
| Matrigel | Corning | 356231 |
| Vitronectin XF | Stem Cell | 07180 |
| | Technologies | |
| Essential 8 ™ Medium | Gibco | A1517001 |
| Versene | Gibco | 15040033 |
| Y-27632 dihydrochloride | Tocris | 1254 |
| (Rho-K inhibitor) | | |
| Recombinant | Bio-Techne | 338-AC-050 |
| Human/Mouse/Rat Activin | | |
| A Protein, 50 ug | | |
| Recombinant Human | Bio-Techne | 7460-F4-025 |
| FGF-4 (aa 71-206) | | |
| CHIR-99021 (CHIR) | TOCRIS | 4423 |
| B27 | Gibco | 17504-044 |
| PerColl | cytiva | 17089101 |
| TaqMan ™ Fast Advanced | ThermoFischer | 4444556 |
| Master Mix | | |
| SYBR | Applied Biosystems | |
| TrypLE-phenol free | Gibco | 12604-021 |
| Ethylenediaminetetraacetic | Fischer | BP2481-100, |
| acid (EDTA) | | CAS 60-00-4 |
| Succinate | Sigma | S3674-100G |
| FITC-4 kDa | Sigma | FD4-100MG |
| DAPI | ThermoFischer | 62248 |
| Hoechst | ThermoFischer | 62249 |
| Texas Red ™-X Phalloidin | ThermoFischer | T7471 |
| Brefeldin A | Invitrogen | 00-4506-51 |
| Monensin | Sigma | M5273 |
| PMA | Sigma-Aldrich | P1585 |
| Ionomycin | Sigma-Aldrich | I0634 |

TABLE 4
Kits used

| REAGENT | SOURCE | IDENTIFIER |
|---|---|---|
| Foxp3/Transcription | Invitrogen | 00-5523-00 |
| Factor Staining Buffer Set | eBioscience | |
| RNeasy | Qiagen | 74106 |
| RevertAid First Strand | ThermoFisher | K1622 |
| CDNA Synthesis Kit | | |
| Live Dead fixable blue/UV | ThermoFischer | L34961 |
| UltraComp eBeads | Invitrogen | 01-2222-42 |

### Examples

### Example 1: Small Intestine Organoids (SIO) Promote Development of ILC from ILCP

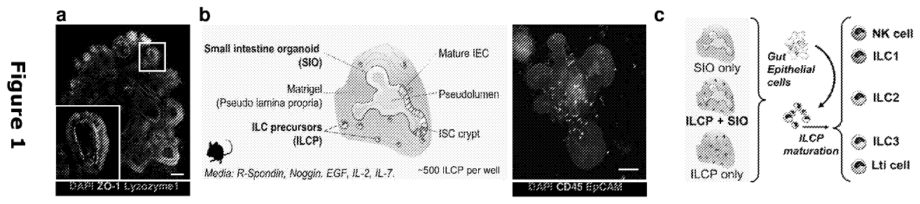

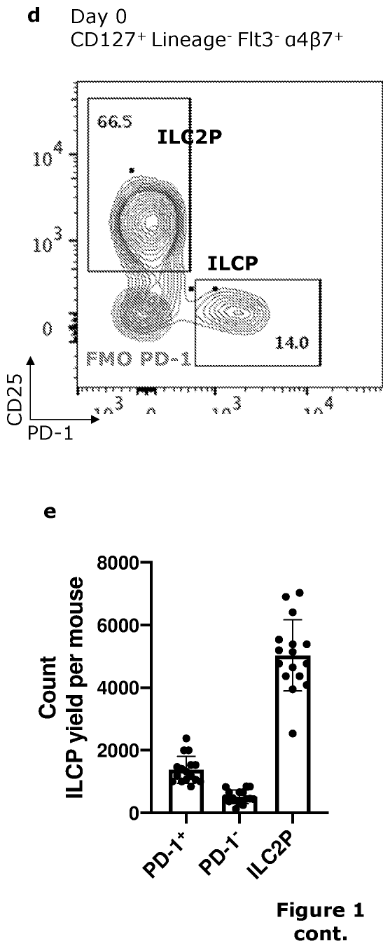

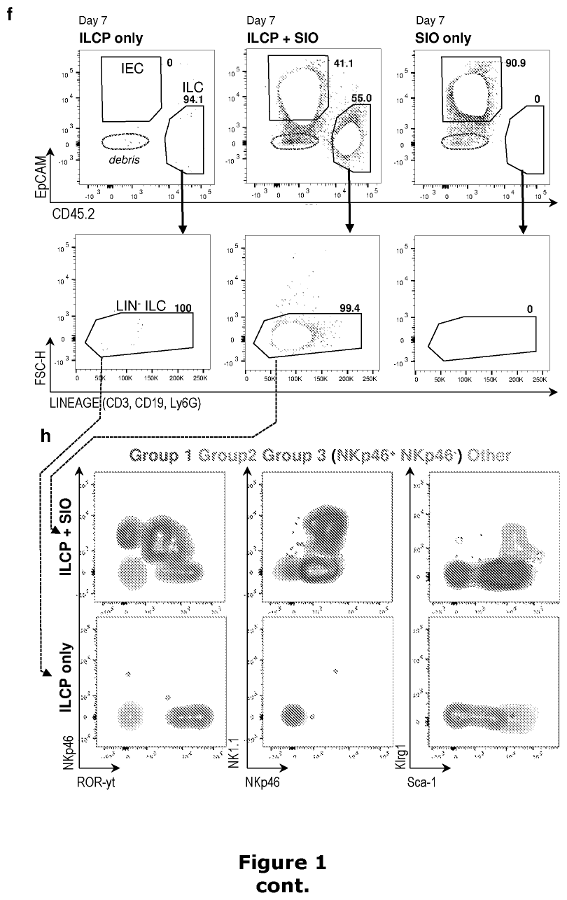

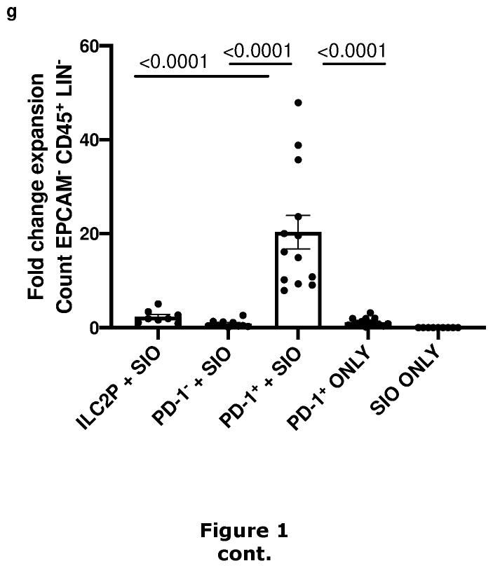

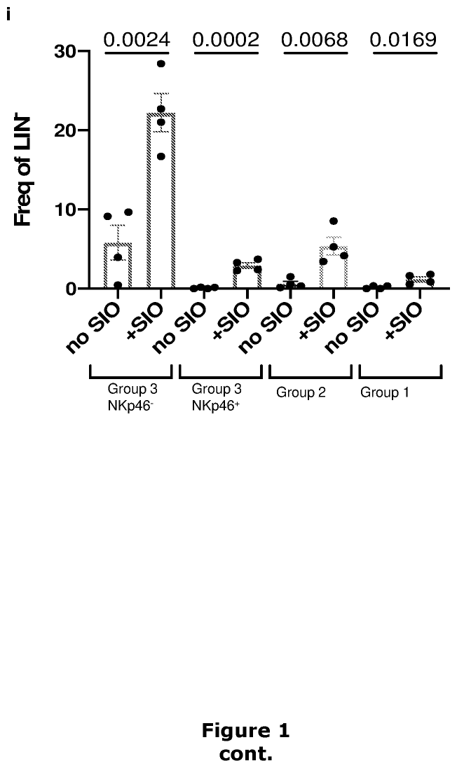

Murine small intestine epithelial organoids (SIO) consist of Lgr5⁺ and Lyzozyme⁺ stem cell crypts (FIG. 1a), which bud into the surrounding extra-cellular matrix and differentiate toward the SIO centre into absorptive and secretory cells. SIO maintain polarity in vitro, with ZO-1⁺ tight junctions creating a contained apical pseudo-lumen (FIG. 1b). To assess the capacity of the basal epithelium to provide a niche for innate lymphoid cells (ILCs), CD127⁺, Lineage⁻, Flt3⁻, α4β7⁺, CD25⁻, PD-1⁺ ILC precursor cells (ILCP) were harvested from adult murine bone marrow, and ~500-1000 ILCP were resuspended and reseeded in 3D Matrigel bubbles either with or without 50-100 intact SIO structures (FIG. 1c-e).

Serially passaged primary SIO cultures are robustly and reproducibly devoid of non-epithelial cells such as immune or mesenchymal cells (Sato et al., 2009). However, as an additional precaution, SIO were also seeded alone to ensure mature ILC were ILCP-derived and not carried over from primary tissue, but no Lineage immune cells were observed in SIO only cultures. Co-cultures were supplemented with EGF (Epidermal Growth Factor), R-Spondin1, and Noggin to support the epithelial crypts, while IL-2 and IL-7 were added to promote survival of ILCP. ILCP moved freely within the Matrigel bubble, where they were exposed to basally presented or secreted epithelial ligands. After 7 days, CD45⁺, Lineage⁻ immune cells were dissociated from EpCAM⁺ epithelial cells using TryPLE for downstream analysis (FIG. 1f). PD-1⁺ ILCP significantly expanded in SIO co-culture (20.33 fold change (FC) from seeded cells), and the presence of epithelial cells was critical for this expansion, as the same culture conditions resulted in an 0.56 FC decrease of CD45⁺, Lineage⁻ immune cells without SIO (FIG. 1g). This system also enabled proliferation of CD25⁺ ILC2P (2.3 FC), whereas the remaining CD25⁻, PD-1⁻ ILC population did not expand, even in the presence of SIO (0.8 FC).

To assess whether SIO promoted maturation (which may otherwise be referred to as differentiation) as well as expansion, ILCP were next derived from isolated from RORγt reporter mice (Rorc^{GFP/+}). The RORγt-ILCP significantly upregulated expression of this group 3 transcription factor when cultured with SIO (FIG. 1h, i). SIO additionally promoted maturation of populations expressing extra-cellular markers associated with type-1 (Klrg1⁻, NKp46⁺, NK1.1⁺) and group 2 ILC (RORγt⁻, Klrg1⁺ILC2). This did not significantly occur with ILCP cultured with IL-2 and IL-7 in Matrigel alone, suggesting that epithelial cells may promote differentiation without stimulation with or supplementation of subset specific cytokines consistently used in other established in vitro protocols (e.g. IL-23, IL-1β, IL-15, IL-12, IL-18, IL-25, and/or IL-33).

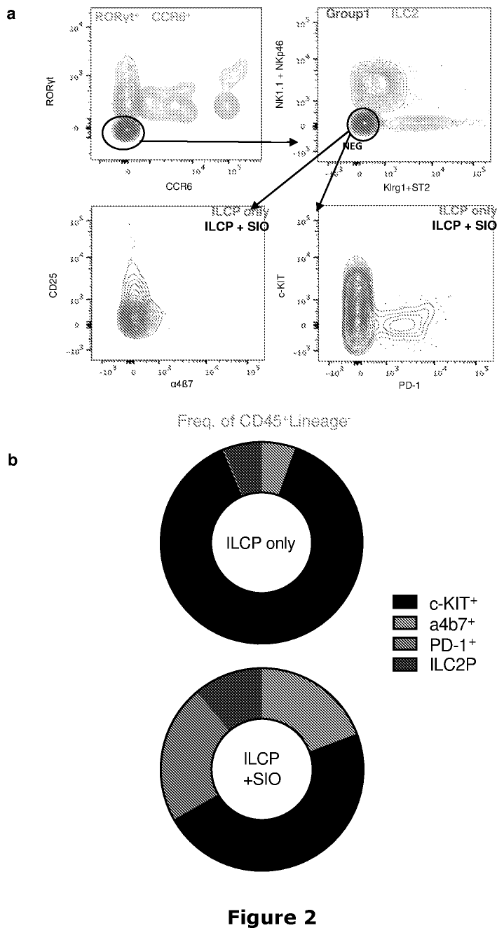

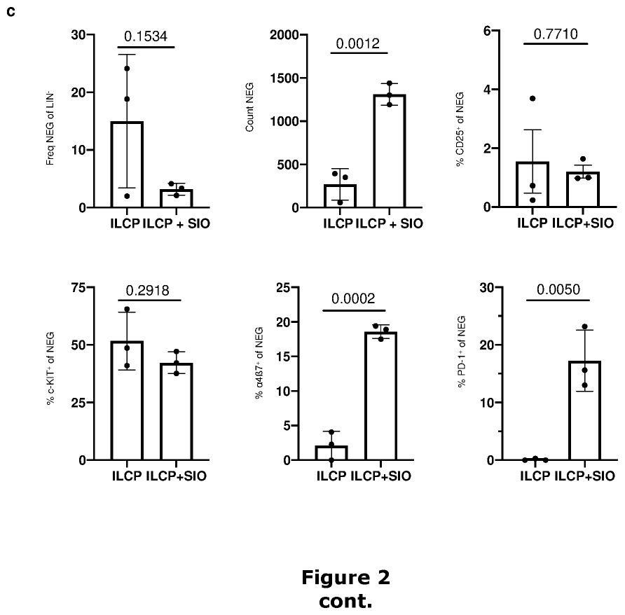

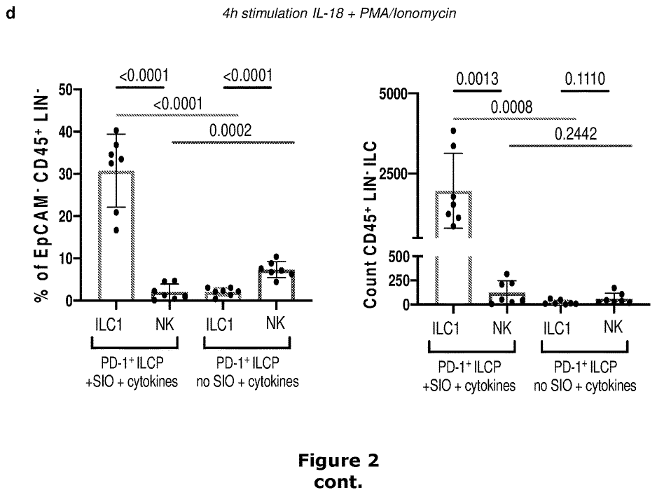

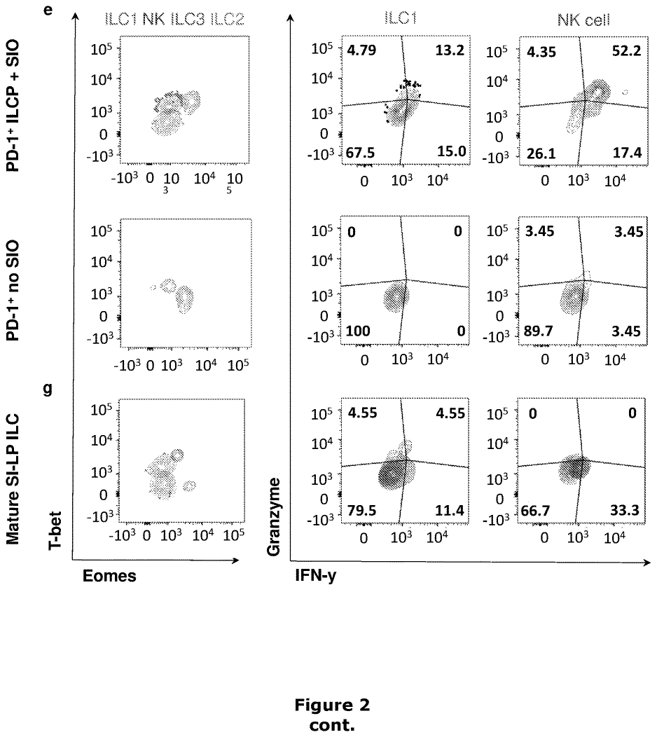

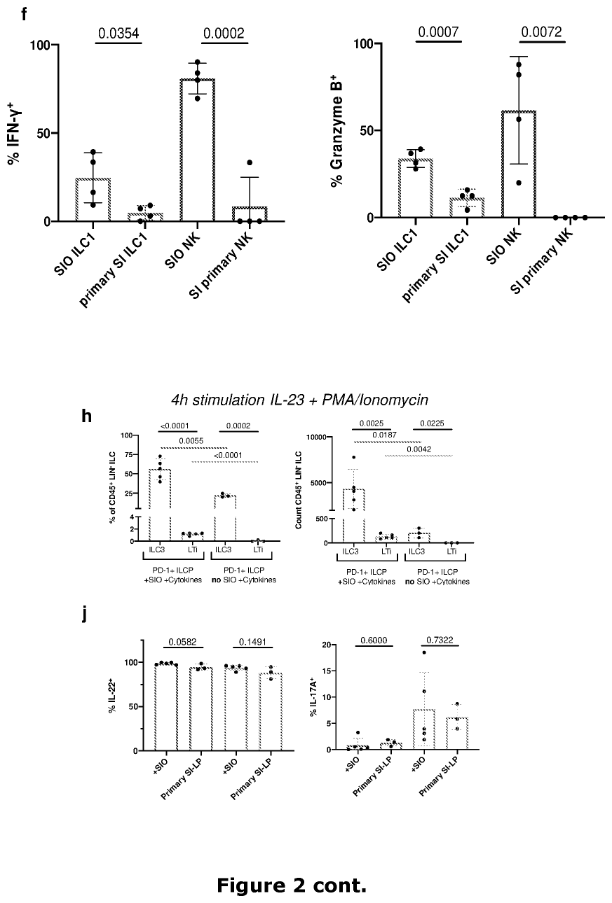

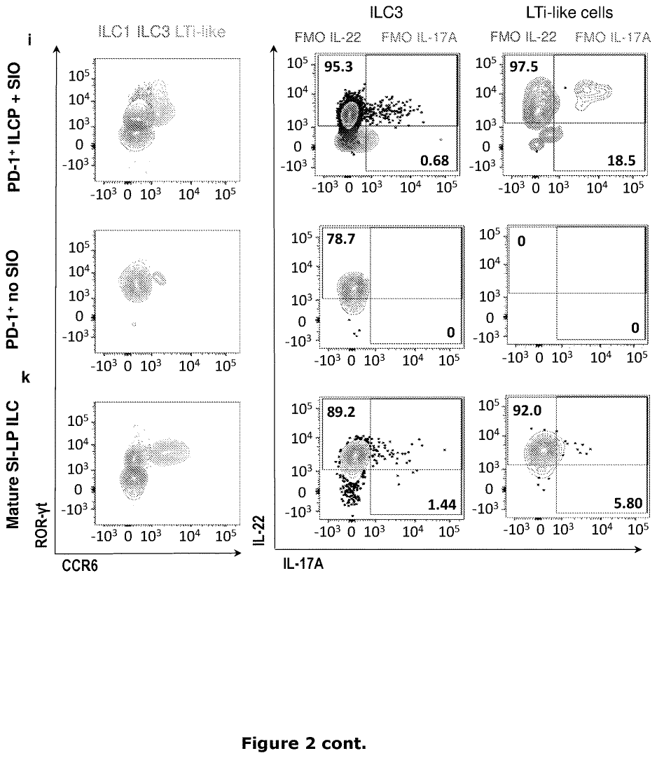

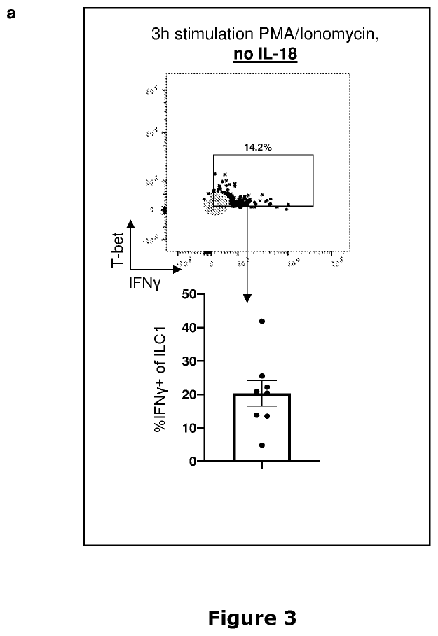

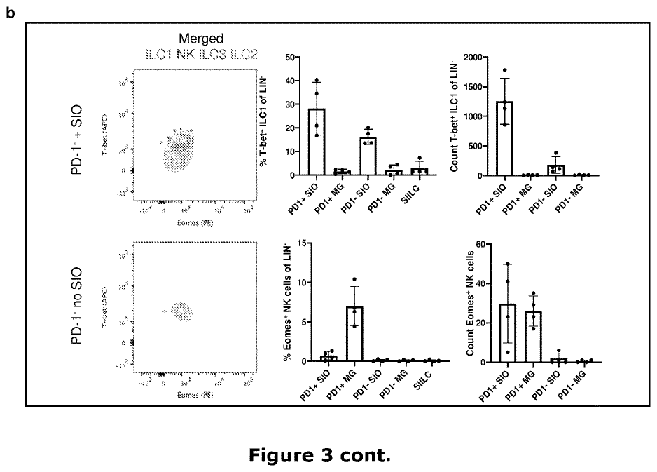

ILCP cultured with or without SIO retained a population of lymphocytes that did not express these maturation markers. To assess the identity of these cells, and to address whether differentiation was actively being promoted or was resulting from repression of precursor stemness, additional markers associated with mature ILC subsets were pooled within the flow cytometry panels (FIG. 2a). The remaining negative population (NEG) broadly expressed ILCP marker c-KIT, and there was no significant difference in the relative proportion of c-KIT⁺ and CD25⁺ NEG cells with or without SIO (FIG. 2a-c). However, the absolute number of NEG cells significantly expanded in SIO co-culture (FIG. 2c), and this population upregulated gut-homing integrin α4β7 and ILCP-marker PD-1. This suggests that adult mucosa have the capacity to actively sustain heterogeneous populations of ILC precursors.

Since maturation was not driven by suppression of ILCP stemness, the functional maturity of putative ILC groups was next examined using group-specific intra-cellular flow cytometry panels. ILCP cultured with SIO produced significantly more RORγt⁻ Klrg1⁻ NK1.1⁺ NKp46⁺ Eomes⁻ T-bet⁺ ILC1 relative to ILCP cultured without SIO (FIG. 2d, e). A small but clear population of T-bet⁺ Eomes⁺ NK cells was also present in co-cultures. There was no significant difference in the absolute number of NK cells between culture conditions. Indeed, the relative proportion of NK cells significantly decreased with SIO (FIG. 2d). When stimulated with non-specific PMA and Ionomycin, ILC1 from these co-cultures demonstrated the capacity to express IFN-γ even without supplementation of IL-12, IL-15, or IL-18. Moreover, a higher proportion of ILC1 and NK cells expressed IFN-γ and Granzyme B upon IL-18 stimulation in co-culture than equivalent group 1 cells isolated from primary small intestine lamina propria, suggesting that precursor derived group 1 ILC were functionally mature (FIG. 2e, f).

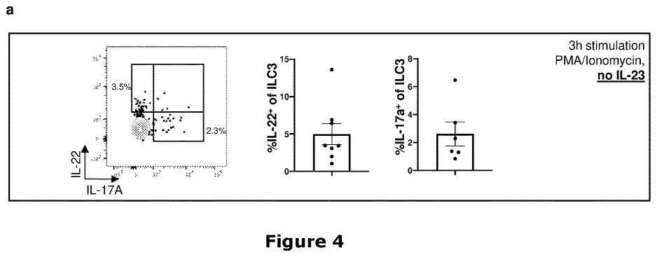

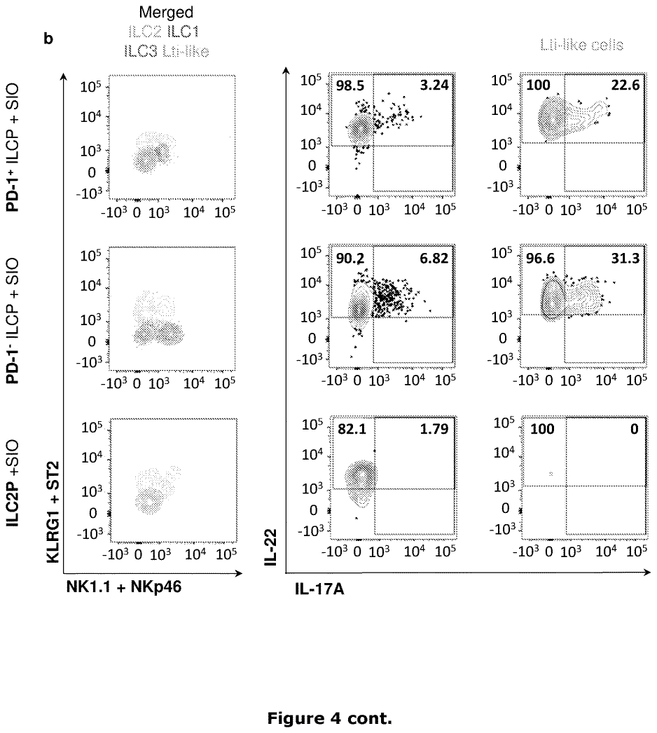

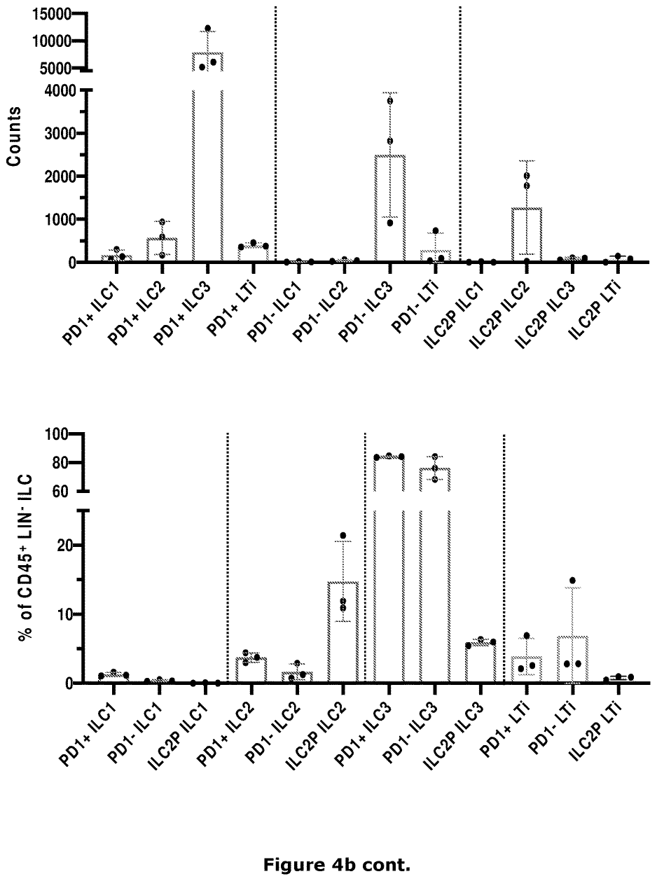

Next, we assessed the identity of the RORγt⁺ population. We observed a small but significant increase in the number of CCR6⁺ RORγt⁺ cells exclusively in SIO culture, suggesting that all ILC subtypes, including LTi-like cells (Klose et al., 2014a), maintain a shared lineage potential from PD-1⁺ ILCP in the correct culture conditions (FIG. 2h, i). After 7 days co-culture, ~5% of RORγt⁺ cells expressed IL-22 and ~2.5% expressed IL-17A upon 3 h unbiased stimulation with PMA/Ionomycin (but not IL-23, FIG. 4a). These cells were additionally highly responsive to subset-specific IL-23 stimulation, with SIO-derived ILC3 secreting IL-22 at comparable rates to primary ILC3. Unlike unbiased PMA/Ionomcyin, this stimulation induced some RORγt and moderate IL-22 expression even in ILCP cultured without SIO, though the absolute number and proportion of maturation was significantly decreased relative to ILCP cultured with SIO (FIG. 2i). Both SIO and primary ILC3 expressed low levels of IL-17A, which was more abundant in CCR6⁺ LTi-like cells of both SIO and primary SI-ILC (FIG. 2j, k). A reduced number of ILC3 and LTi-like cells were present in some PD-1⁻ co-cultures with, but not without SIO (FIG. 4b). Finally, ILC2 precursors, which predominantly gave rise to putative ILC2, only yielded negligible ILC3 numbers and no LTi-like cells.

### Example 2: SIO Recapitulate ILC Subset Ratios Characteristic of the Small Intestine

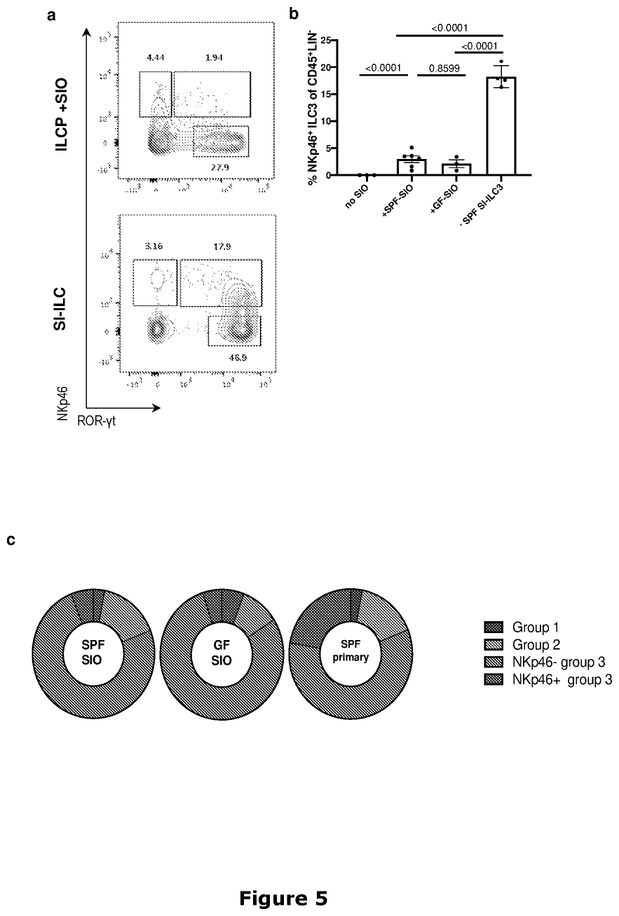

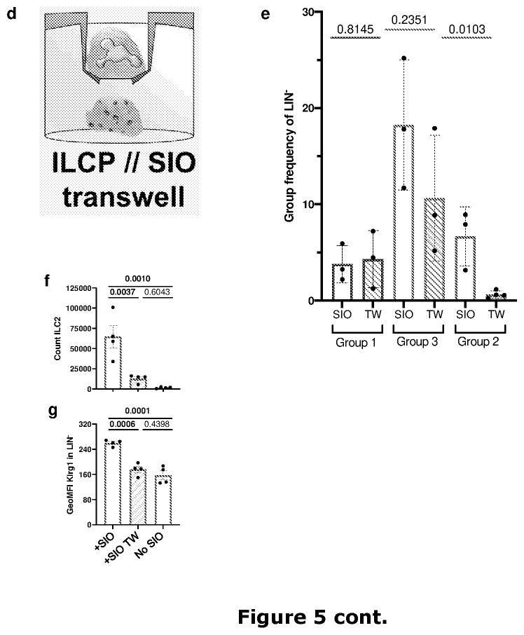

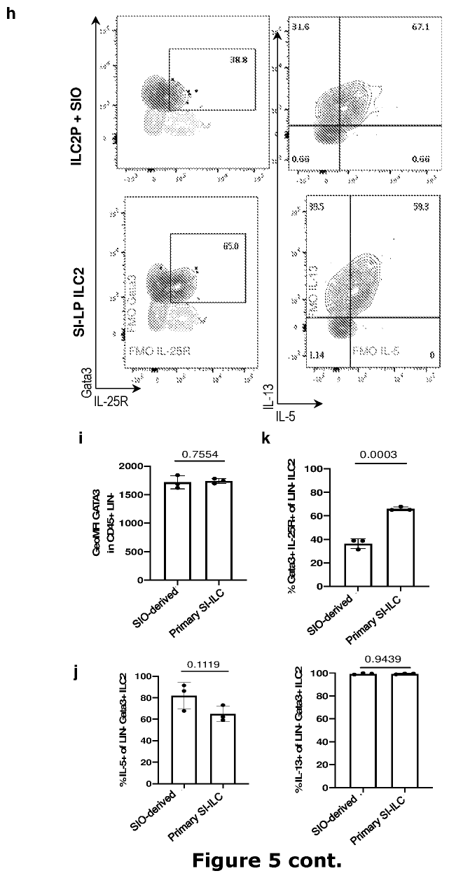

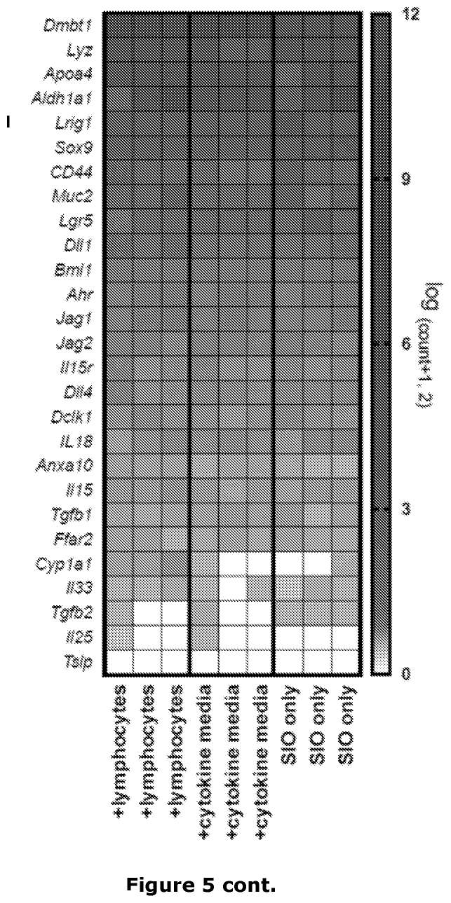

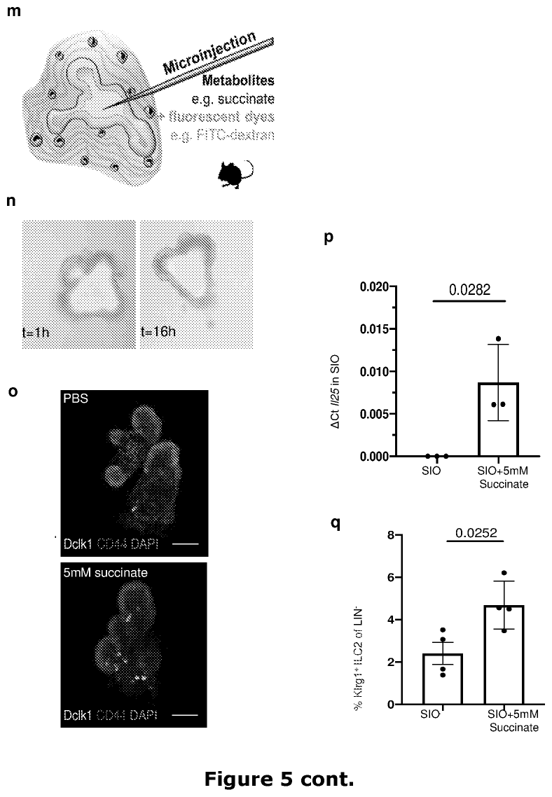

Although the production of cytokines in SIO-derived ILC3 rivalled that of primary ILC3, only ~3% of this RORγt⁺ population expressed Natural Cytotoxicity Receptor (NCR) NKp46, representing a significantly smaller ratio of this population when compared to ~18% NKp46CR⁺ ILC3 in primary SI (excluding Peyer's Patches) (FIG. 5a). It has previously been reported that NKp46 expression in ILC is in part regulated by the microbial landscape, which may impact expression of epithelial Notch ligands (DLL1, JAG1, JAG2) that may be essential but not sufficient for the final maturation of adult NKp46⁺ ILC3s. Intestinal organoids can maintain the epigenetic signatures of the donor tissue from which they were derived. However, whether serially passaged organoids resemble germ-free (GF) epithelium in vitro or retain epigenetic signatures of previous specific pathogen free (SPF) microbial exposure remains somewhat contested. To address this question, SIO were additionally derived from GF animals, and cultured with RorceGFP ILCP. Both SPF and GF SIO yielded predominantly NKp46-ILC3, and did not significantly differ in their capacity to drive ILC maturation, suggesting that SPF-derived SIO provide an essentially GF model of the epithelium (FIG. 5b).

Although NKp46 expression differed within primary and SIO-derived ILC3, it is notable that the overall distribution of mature ILC groups yielded from unstimulated SIO cultures was similar to the overarching tissue-specific ratio of ILC subsets observed in the homeostatic primary small intestine, which is highly enriched for group 3 ILC during homeostasis (FIG. 5c). To evaluate whether ILCP required contact with the epithelium to induce this gut-specific group distribution, ILCP were separated from SIO using a transwell insert (TW). This did not affect group 1 and group 3 ILC, but significantly reduced in the number of putative ILC2 (FIG. 5d, e). These experiments were repeated with CD25⁺ ILC2P, as their relative abundance within the murine bone marrow (FIG. 1d) allowed for separation into more experimental conditions from the same animal. The loss of RORγt⁻ Klrg1⁺ cells was recapitulated with ILC2P, with the frequency of this population (FIG. 5f) and the expression of Klrg1 (FIG. 5g) significantly decreasing when cultured either without SIO or with SIO separated by a transwell insert. To delve further into the necessary and potentially tissue-specific interactions between gut epithelial cells and ILC2, the maturity of ILC2P-derived ILC2 was next compared to primary ILC2, as previously demonstrated for the other ILC subtypes. Co-culture and primary tissue-derived ILC2 did not differ in their expression of type 2 transcription factor Gata3 (FIG. 5h, i), and when stimulated ~80% of ILC2 expressed IL-5, and almost all ILC2 expressed IL-13, showing no significant differences in cytokine expression to primary ILC2 (FIG. 5j).

Much like the expression of NKp46 was reduced in SIO-derived ILC3, expression of IL-25R characteristic of intestinal ILC2 was significantly decreased in co-cultures relative to primary intestinal ILC2 (FIG. 5k). To assess what epithelial stimuli were present lacking to drive this maturation, EpCAM⁺CD45⁻ epithelial fraction of unstimulated common ILC precursor co-cultures with SIO, and of SIO cultured without lymphocytes in media with or without IL-2 and IL-7 were characterised by bulk RNA-sequencing (FIG. 5l). Targets associated with ILC maturation included genes for expression of Notch ligands DII1, DII4, Jag1, and Jag2, cytokines IL-15 and IL-18, and basally secreted growth factors from Bmp, Wnt, Fgf, and Tgf families. None of these genes of interest were significantly differentially expressed between culture conditions, nor were transcriptional signatures associated with small molecule biosynthesis (including genes for ILC3-associated retinoic acid (RA) synthesizing enzyme aldh1a1. Without wishing to be bound by theory, this may suggest that epithelial cells have a steady-state capacity to promote ILC maturation that is independent of and/or precedes the presence of immune cells.

SIO expression of Tuft-cell derived IL-25R/IL-17RB ligand Il25 was either low or absent in all conditions. In adult animals, it is reported that expression of epithelial-derived cytokines can be modulated by luminal metabolites, for instance succinate contributes to IL-25/IL-17E expression in Tuft cells. The absence of IL-25 in SIO provided an optimal template to assess the capacity for these essentially GF-organoids to recapitulate this well-established Succinate-Tuft-ILC2 circuit. We harnessed the closed compartmental nature of organoids, which allow microinjection of components into the pseudo-lumen (FIG. 5m). The epithelial integrity of this compartment was demonstrated by the retention of the 204 kDa molecule Fluorescein isothiocyanate-Dextran (FITC) (FIG. 5n). When injected with succinate, the number of Dclk1⁺ Tuft cells increased in SIO (FIG. 5o) correlating with increased expression of Il17e/Il25 (FIG. 5p). Moreover, ILC precursors co-cultured with the succinate-injected SIO yielded significantly more Klrg1⁺ ILC2s, suggesting that despite low-level expression of IL-25R, SIO-derived ILC2 retain the capacity to proliferate in response to epithelial-derived IL-25 (FIG. 5q).

### Example 3: Epithelial Identity Drives Tissue-Specific ILC2 Phenotypes

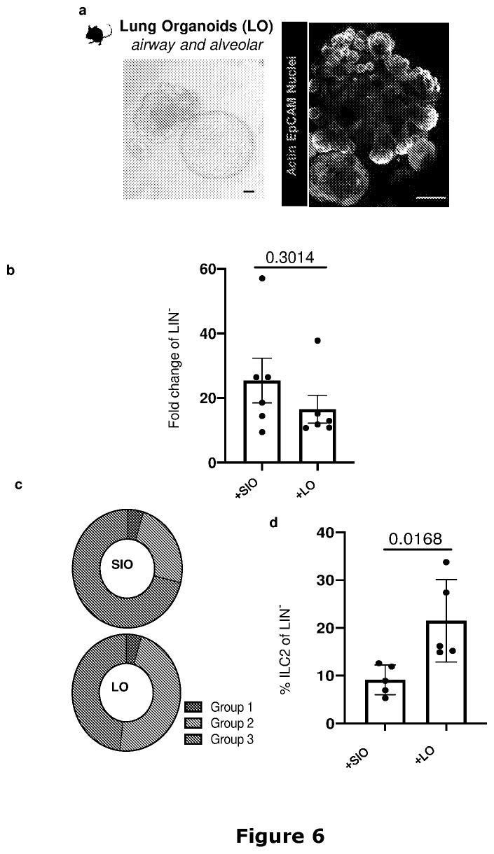

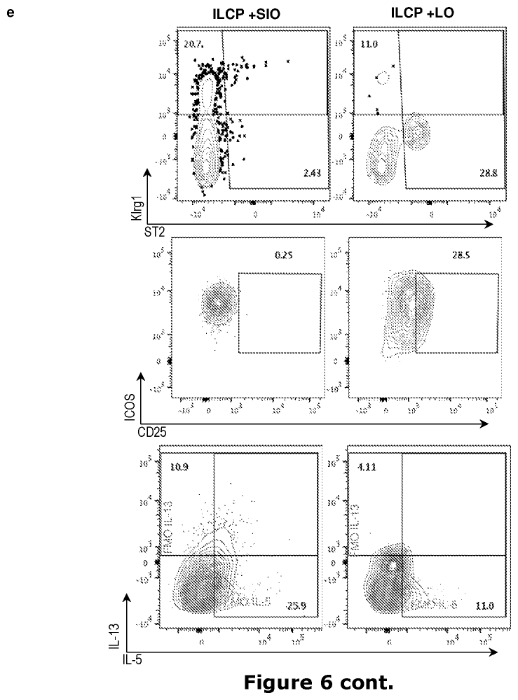

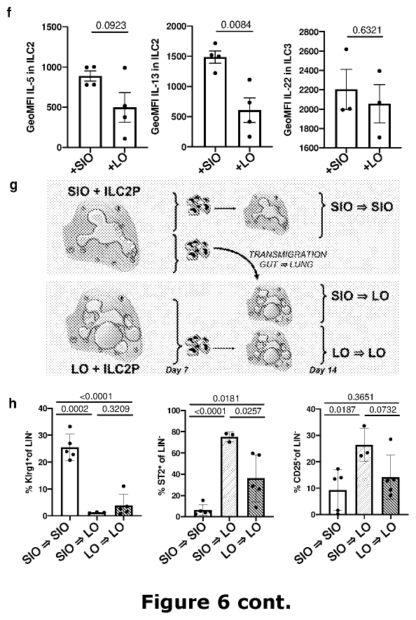

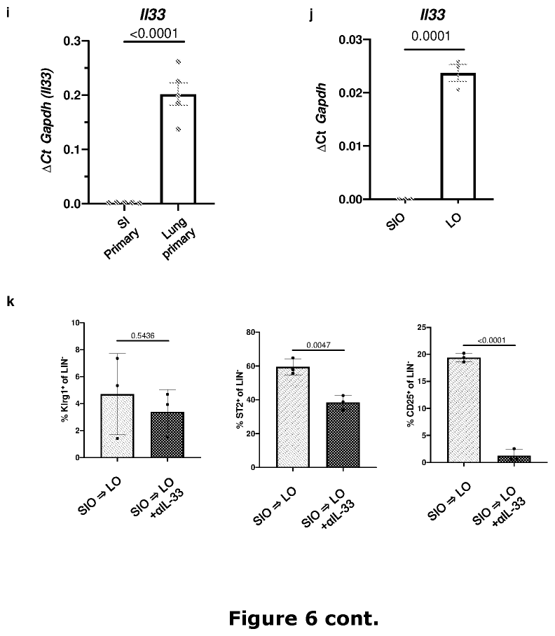

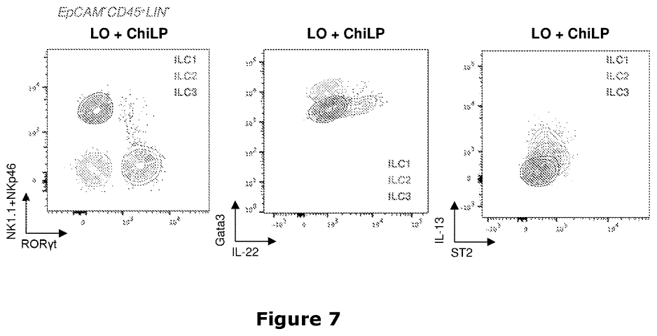

ILC3 are the dominant population in the adult murine small intestine, whereas ILC2s are relatively more abundant in the post-natal murine lung (Dutton et al., 2018; Saluzzo et al., 2017). To assess whether epithelial identity alone was sufficient to induce the development of this “anticipatory” ILC2 imprint from bone marrow ILC precursors, we derived murine lung organoids (LO) from adult murine EpCAM⁺ enriched primary lung tip epithelial cells, which yielded a mix of “cystic” round organoids, and “saccular” organoids made up of small budding structures (FIG. 6a). Cystic lung organoids consist of airway epithelial cells, rich in Club, Goblet, and ciliated cells, whereas the saccular structures are rich in surfactants, and contain the alveolar AEC1 and AEC2 cells that enable gas exchange (McQualter et al., 2010). Thus, much like SIO, LO provide a rich snapshot of the many cell types of a complex lung epithelium. Much like SIO, LO also supported expansion (FIG. 6b) of putative group 1, group 2, and group 3 ILC from Common helper-like ILC precursors (FIG. 6c, FIG. 7). Notably, the relative frequency of EpCAM⁻, CD45⁺, Lineage⁻, RORγt⁻, NKp46⁻, NK1.1⁻, Gata3⁺ ILC2 significantly increased in the lung organoid co-cultures. Indeed, these ILC2 recapitulated tissue-specific features that distinguish murine gut and lung ILC2 in vivo (GSE117568, (Ricardo-Gonzalez et al., 2018a)) (FIG. 6d). Despite an absence of other immune cells, or stromal cells, or tissue-specific microbiota, epithelial-only SIO co-cultures yielded a higher proportion of Klrg1⁺ ILC2 than the LO, whereas LO-derived ILC2 expressed higher levels of IL-33 receptor ST2 (FIG. 6e). Finally, while ILC3 from these co-cultures did not differ significantly in their expression of IL-22, ILC2 in the gut appeared to express higher amounts of type-2 cytokines, with IL-13 being significantly enriched in SIO-derived ILC2, as predicted from meta-analysis of RNA-expression in murine primary gut and lung ILC2 (FIG. 6f).

These tissue specific ILC2 patterns become pertinent in murine pulmonary helminth infections. In this context, Klrg1^{high} “inflammatory” iILC2 (Huang et al., 2015) expand in response to IL-25, then transmigrate from the gut to the lung to clear infection (Huang et al., 2018). Indeed, only adoptive transfer of these iILC2-rich gut ILC—but not of Klrg1^{low} “natural” nILC2-rich lung ILC or of bone-marrow ILCP-results in successful pathogen clearance. The ultimate fate of gut iILC2 in the lung is not known, as Klrg1^{high} ILC2 are no longer detected in the lung after resolution of infection. To address these phenomena in the reductionist organoid system, ILC populations were isolated from SIO by FACS after 7-day co-culture, then re-seeded with fresh LO, effectively recapitulating post-migrational dynamics of this population upon reaching the lung (FIG. 6g). After 7 additional days of co-culture, a significant downregulation of Klrg1 was observed in previously Klrg1^{high} SIO-derived ILC2 (FIG. 6h), suggesting that these may become poorly distinguishable from Klrg1^{low} lung/nILC2. Conversely, ST2 and CD25 were significantly upregulated by SIO-ILC2 upon transfer to LO. Interestingly, upregulation of ST2 was significantly higher in SIO-LO-swapped ILC even when compared to LO-derived ILC that were re-seeded with LO. This suggests that ILC2 adopt an imprint of intestinal origin. This may make them more receptive to pulmonary stimuli than nILC2, offering a potential explanation for their unique capacity to clear pulmonary helminth infections.

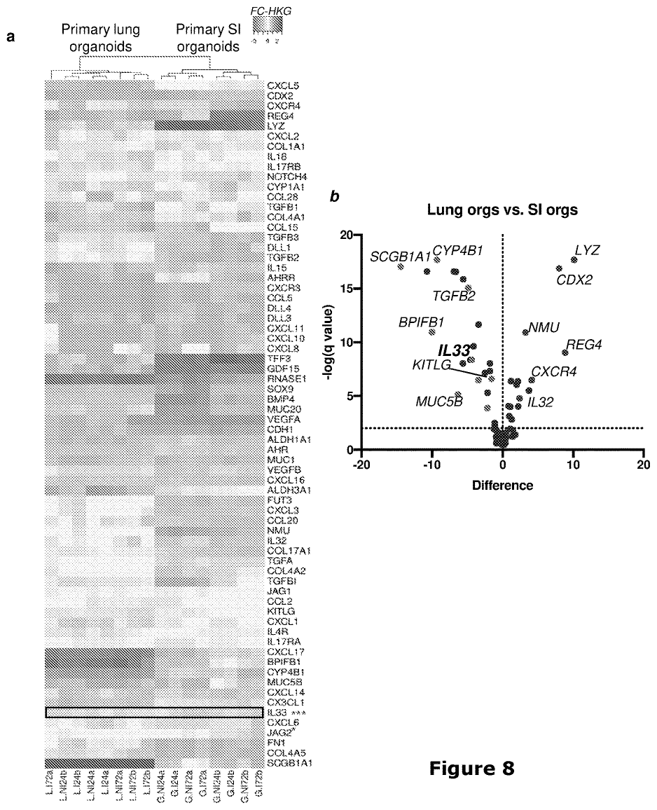

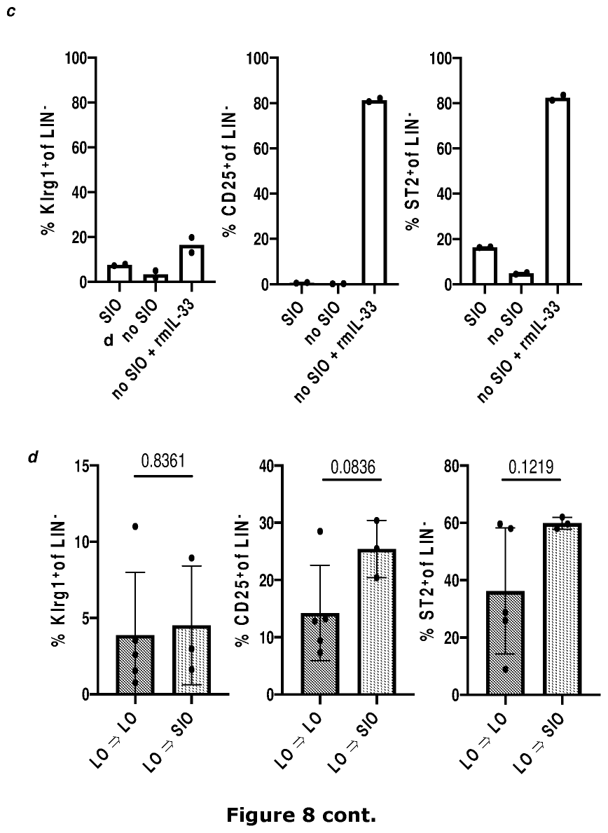

To understand how gut and lung epithelium differed in expression of previously addressed target genes (FIG. 5l), a meta-analysis of publicly available and comparable primary adult human gut and lung epithelial-only organoid gene expression was performed GSE112991 (Heo et al., 2018); FIG. 8a, b). In this dataset, lung and gut epithelial cells clustered separately, recapitulating anticipated tissue-specific expression of transcripts for intestinal antimicrobial peptides (e.g. LYZ, REG4) and pulmonary surfactant proteins (e.g. SCGB1A1). As in the murine organoids, IL17E/IL25 was not detected in either gut or lung human organoids, and as in the SIO cultured with or without lymphocytes and immune cytokines, the majority of basally presented or secreted genes of interest were not significantly differentially regulated between the two epithelia. However, expression of ST2-ligand IL-33 was significantly enriched in pulmonary epithelial cells (FIG. 8b). This differential expression was conserved in primary murine small intestine and lung tissue (FIG. 6i), as well as in the SIO and LO cultures used in these experiments (FIG. 6j). To assess whether the role of this tissue-specific difference in mediating the phenotypic transformation of gut-derived ILC2, a neutralising dose of murine anti-IL33 antibody was supplemented to gut-to-lung swapped co-cultures. Inhibition of IL-33 had no impact on Klrg1 downregulation, however it significantly dampened upregulation of ST2 and CD25 (FIG. 6k). To ensure that these changes are actively induced by pulmonary epithelial cells-including IL-33- and not merely from a loss of some intestinal-specific repressive stimuli, SIO-derived ILC were additionally reseeded in Matrigel without SIO or LO, but with or without recombinant murine IL-33 (FIG. 8c). A loss of contact with SIO was not sufficient to recapitulate the SIO-to-LO phenotypes, suggesting that ST2 and CD25 expression are not repressed by an intestinal milieu. Instead, recombinant IL-33 alone was sufficient to induce high levels of ST2 and CD25 expression in SIO-matured ILC (FIG. 8c). Finally, when LO-matured ILC were swapped to SIO, no significant differences in CD25, ST2, or Klrg1 expression was observed (FIG. 8d).

### Example 4: Human Intestinal Organoids Promote Maturation of Human Systemic ILCP

The translation of experimental approaches used in mice to distal human tissues, especially when studying rare populations like ILC, can be challenging. This makes the potential of in vitro human mucosal organoid systems particularly appealing. However, human bone marrow ILC precursors are poorly accessible. Thus, to translate the previously described murine ILCP-organoid system, readily available systemic ILCP were isolated from peripheral blood mononuclear cells (PBMC) instead of bone marrow, while human intestinal organoids (HIO) were derived from healthy KUTE-4 human induced pluripotent stem cells (McCracken et al., 2011). These GF structures are well-established and can be matured through addition of IL-2.

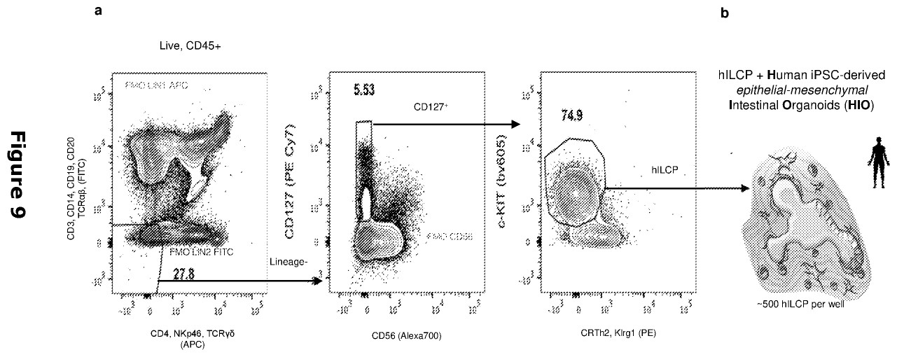

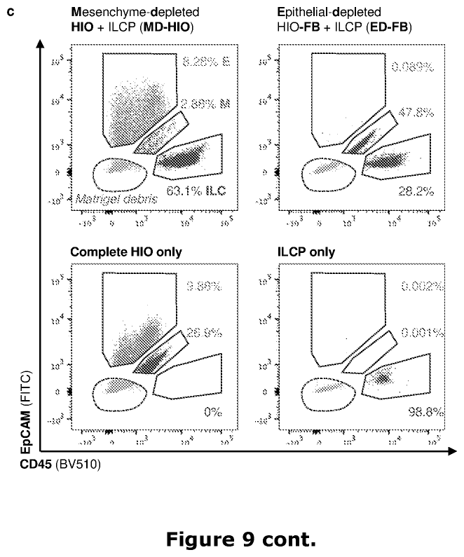

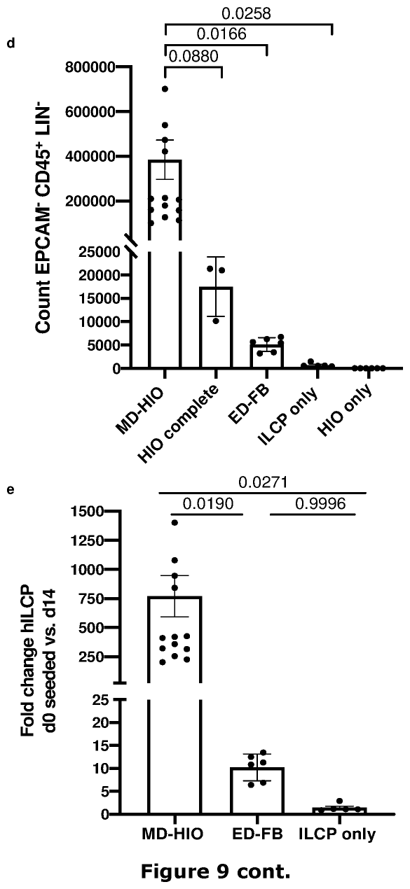

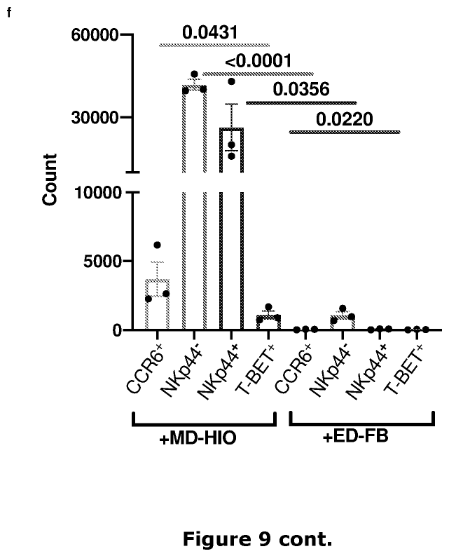

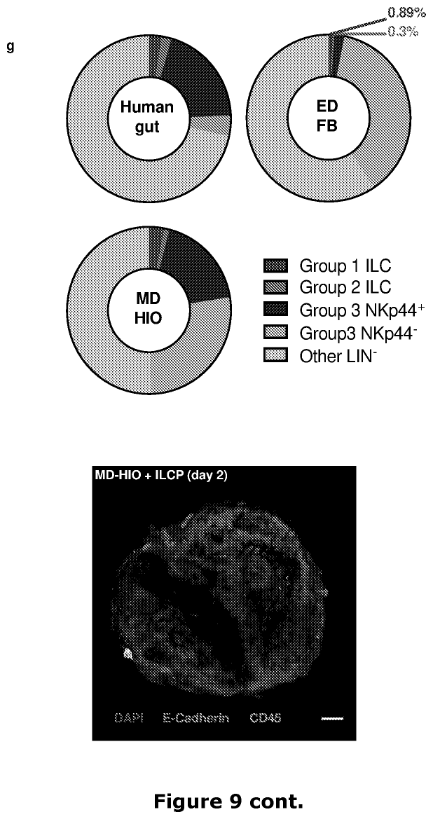

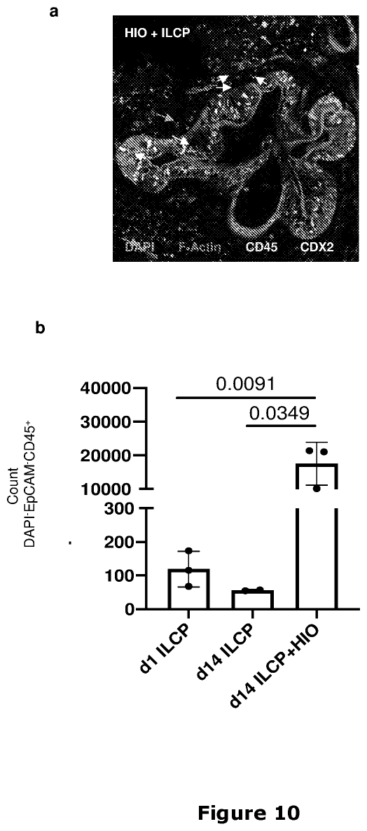

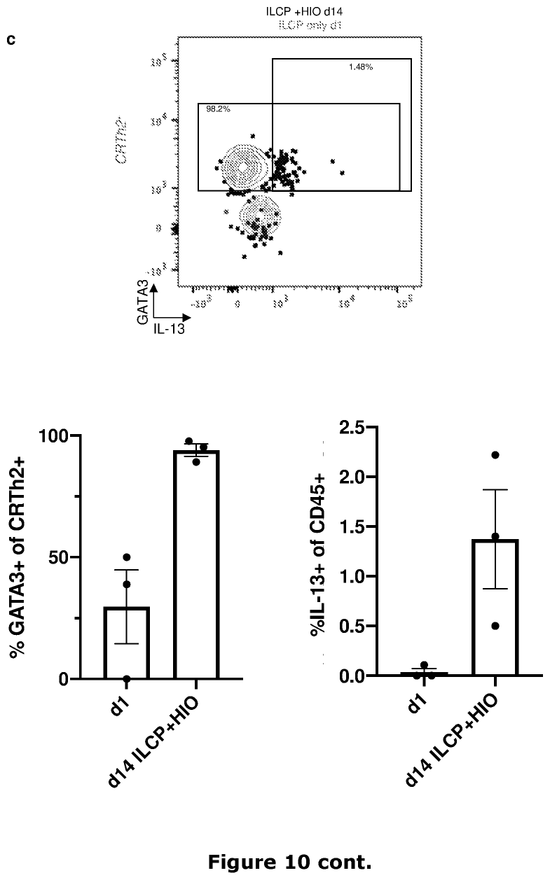

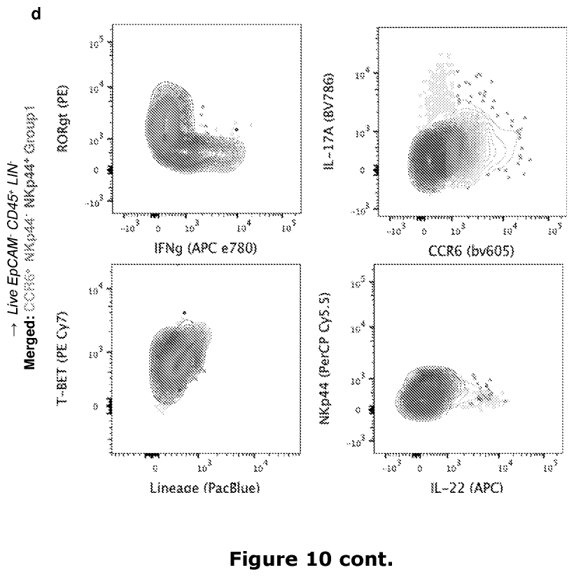

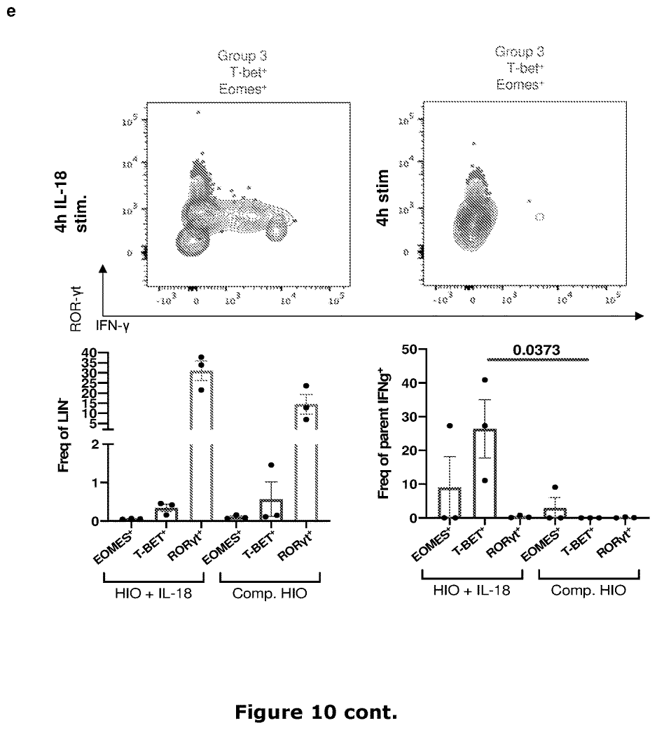

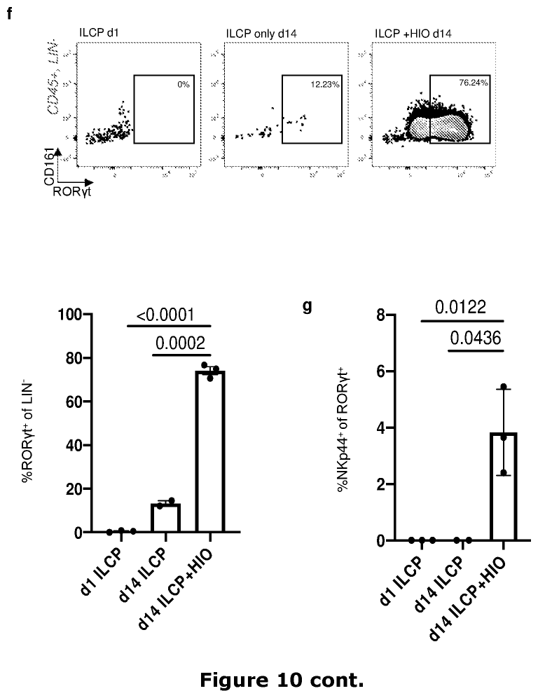

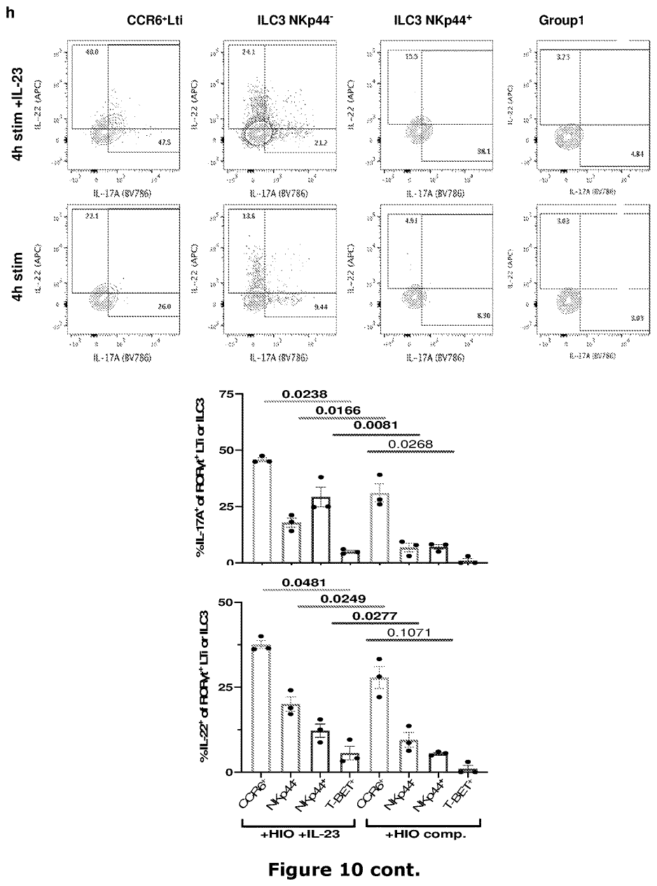

Heterogeneous systemic ILCPs (FIG. 9a) were co-cultured with these hiPSC-derived epithelial-mesenchymal HIO (FIG. 9b). Co-culture with HIO significantly expanded (~20 fold) ILCPs relative to the number of cells seeded without HIO, and when compared to the number of ILCP seeded on day 1 (d1) (FIG. 10a, b). As in murine co-cultures, human ILCP developed as putative group 2 (FIG. 10c), group 3 (FIG. 10d), and group 1 ILC (FIG. 10e) when cultured with whole HIO supplemented with IL-2 and IL-7. This fold expansion and development occurred without requiring the addition of IL-1β or subset-specific cytokines, whereas previous in vitro systems required supplementation with IL-1β+IL-23 for ILC3s, IL-25+IL-33 for ILC2s, and IL-12+IL-18 for robust ILC1 development (Lim et al., 2017b), or of IL-15 and differential Notch ligands when matured from CD34⁺ hematopoietic stem cells (Hernandez et al., 2021). Instead, HIO alone significantly supported maturation of RORγt⁺ ILC FIG. 10f) which significantly upregulated NKp44 (FIG. 10g), recapitulating the difference in c-KIT⁺ ILC observed between periphery and gut by Lim and colleagues. These NKp44^{+/-} CCR6^{+/-} group 3 RORγt⁺ ILC produced low levels of IL-22 and IL-17A upon non-specific PMA/Ionomycin stimulation, and significantly responded to additional 4 h stimulation with IL-23 (FIG. 10h), while putative group 1 ILC significantly upregulated expression of T-bet and IFN-γ secretion in response to 4 h stimulation with IL-18 (FIG. 10e).

### Example 5: Epithelial Cells, not Mesenchyme, Drive Robust ILC Expansion and Maturation

These data suggest the human intestinal organoid microenvironment provides stimuli for baseline maturation of ILC subsets from ILCP. However, unlike the murine primary epithelial-only organoids, hiPSC-derived hindgut organoids co-develop with rich and complex native mesenchyme. These cells not only contribute to the maturation of epithelial cells (Stallmach et al., 1989), but are a known source of ILC-survival factor and CD127-ligand IL-7 (Xu et al., 2015). To assess whether epithelial cells or mesenchymal cells were predominantly driving this ILC maturation, the matrix of HIO structures was digested with Collagenase, and a single cell suspension of mesenchyme was separated from 3D epithelial structures through serial gravity-gradient separation, resulting in a mesenchyme-depleted HIO fraction (MD-HIO) and an epithelial-depleted fibroblast-rich fraction (ED-FB) (FIG. 9c). These fractions were cultured with ILCP for 14 days, and both fractions were able to maintain ILC viability and expansion (FIG. 9c). However, the mesenchyme-depleted epithelial fraction unexpectedly yielded a dramatic increase in EpCAM⁻, CD45⁺, Lineage cells after culture (FIG. 9d), significantly increasing the fold-change expansion of seeded ILCP relative to those seeded with epithelial-depleted mesenchyme or without organoids in Matrigel only (FIG. 9e). Moreover, while complete epithelial-mesenchymal HIO induced NKp44 expression in ~2-6% of group 3 ILC (FIG. 10g), this upregulation was significantly greater in mesenchyme-depleted co-cultures (15-22%) (FIG. 9f, 11a). Indeed, the epithelial-enriched MD-HIO fraction promoted patterns of ILC-subset maturation that more closely resembled the distribution of mature ILC in the healthy human intestine (Krämer et al., 2017) than the epithelial-depleted mesenchyme fraction (FIG. 9g). As with expression of NKp46 in the GF murine model (FIG. 5a), the relative proportion of NKp44⁺ to NKp44⁻ putative ILC3 biased maturation towards an NCR population in GF, environmentally controlled MD-HIO (FIG. 9f, g), in which ILCP could freely interact with the epithelium (FIG. 9h).

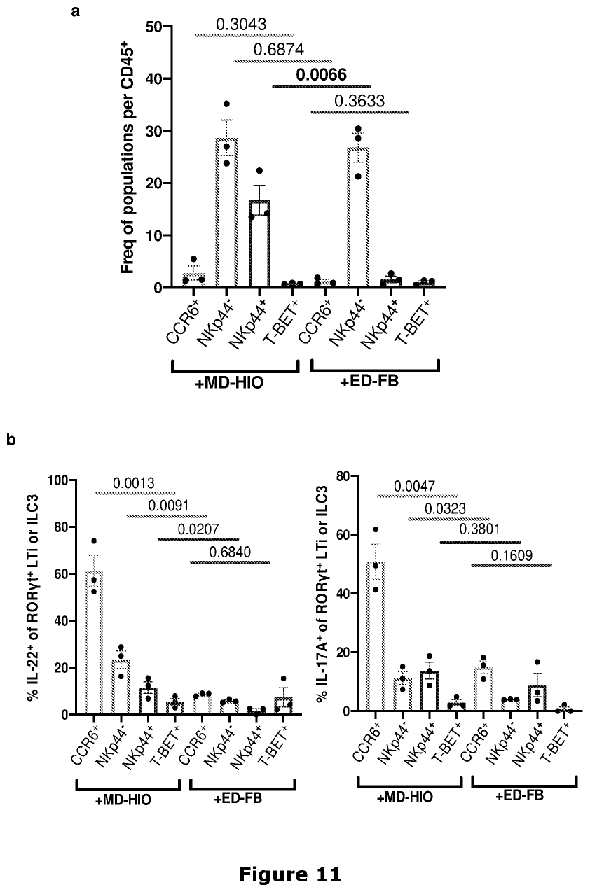

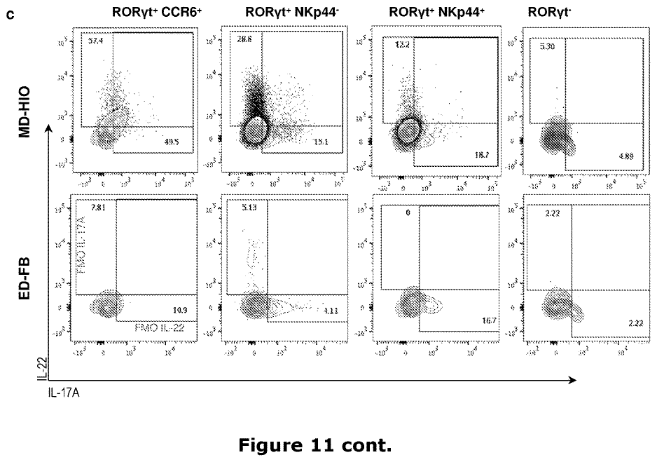

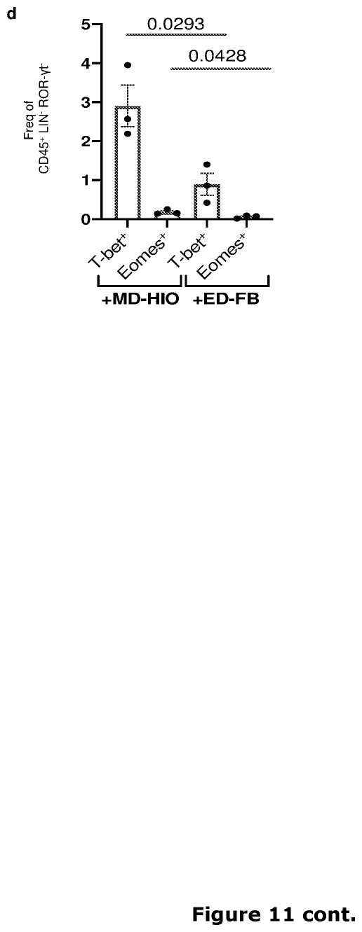

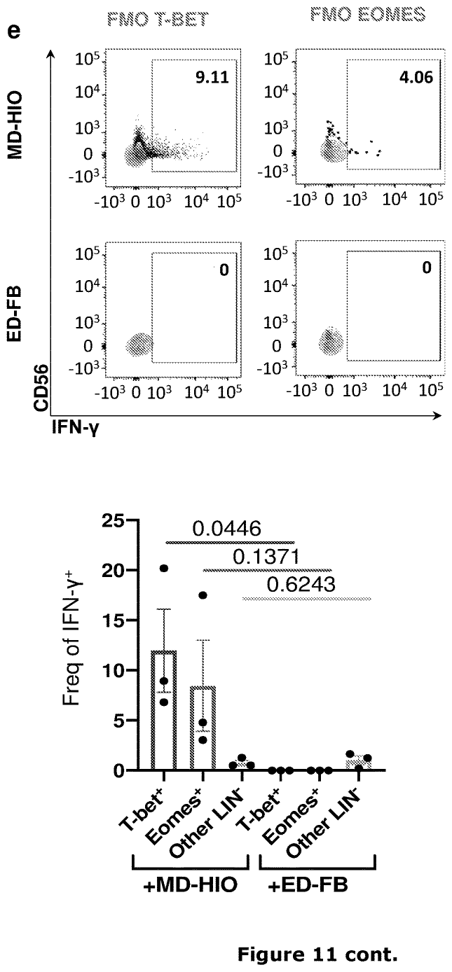

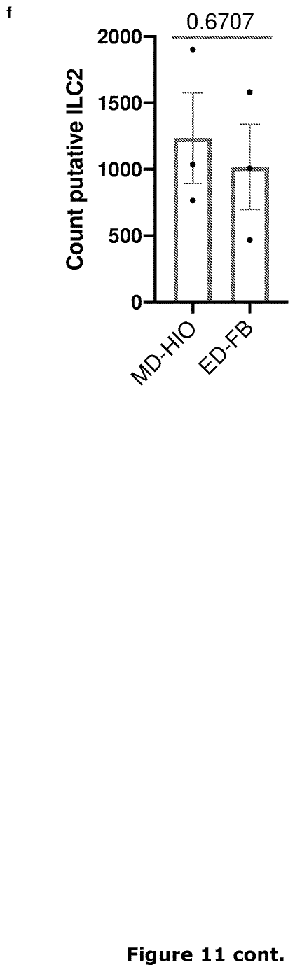

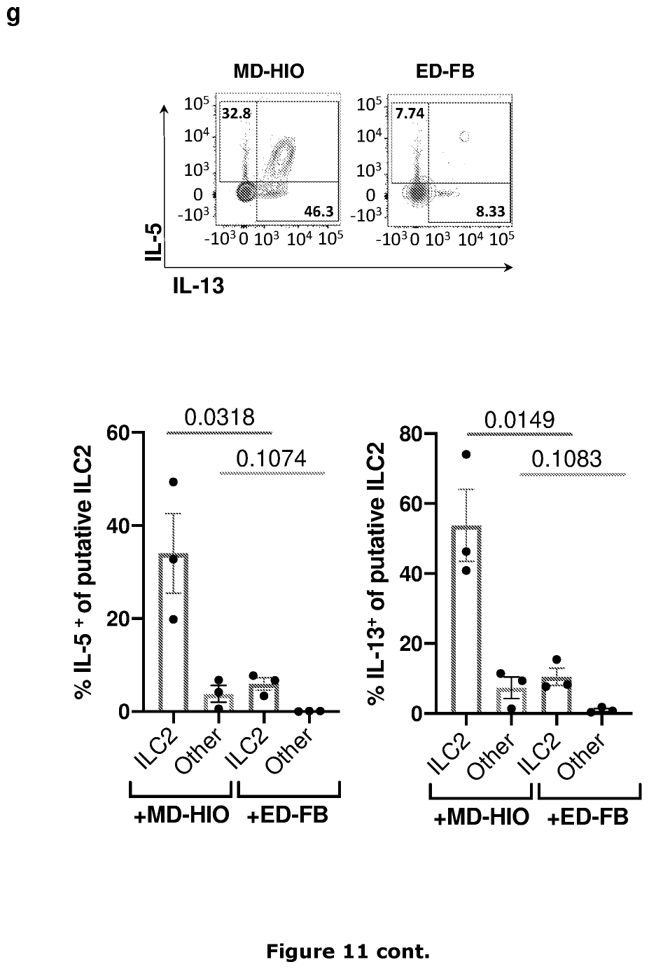

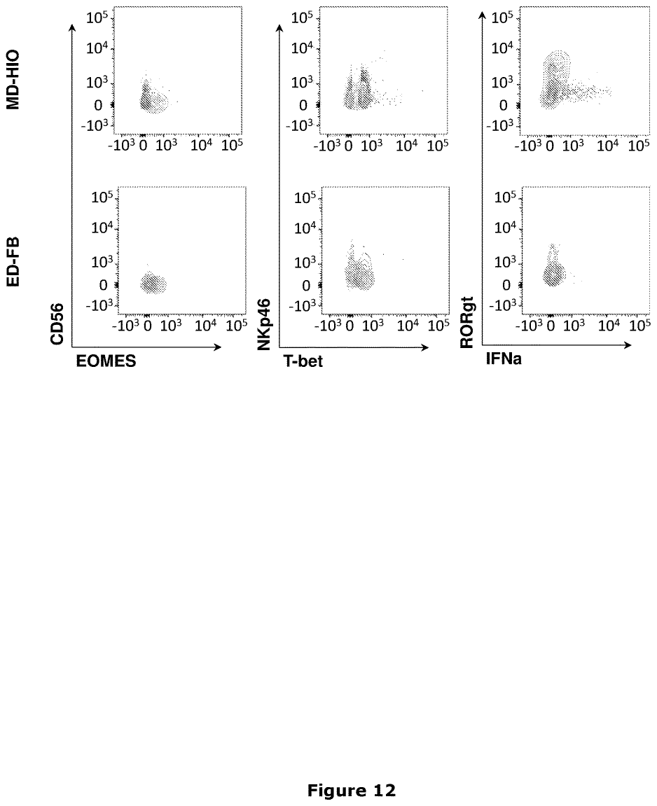

The current gold standard for investigating murine and human ILC maturation in vitro relies on modified murine bone marrow stromal cells (Nakano, 1996). Therefore, the relatively poor ILCP expansion rates induced by hiPSC-derived intestinal mesenchyme were unexpected. We hypothesized that the increased cell number in mesenchyme-depleted HIO could represent proliferation of poorly differentiated, immature ILC, while stromal cells may promote a smaller but more mature ILC yield. To test this, unbiased stimulation with PMA/Ionomycin was performed in ILC derived from MD-HIO and ED-FB co-cultures. First, the maturation of putative Group 3 ILCs was assessed. Here, only the frequency of NKp44⁺ cells of RORγt⁺ ILC significantly expanded in MD-HIO, with the relative frequency of CCR6⁺, NKp44⁻, and T-bet⁺ ILC remaining constant in both conditions (FIG. 11a). However, CCR6⁺ and NKp44+/−ILC expressed significantly greater amounts of IL-22, and CCR6⁺ and NKp44⁻ ILC expressed significantly more IL-17A than did the respective corresponding populations derived from the epithelial-depleted mesenchymal fraction (FIG. 11b). In fact, the frequency of IL-22⁺ ILC was either comparable or greater in the mesenchyme-depleted fractions with unbiased PMA/Ionomycin stimulation than in complete HIO co-cultures stimulated with IL-22-inducing IL-23 (FIG. 10h). The proportion of putative group 1 CD56^{+/-}, T-bet⁺ and Eomes⁺ ILC (FIG. 12) was not only significantly decreased in the epithelial-depleted fraction (FIG. 11d), but no expression of IFN-γ was observed in either population in the absence of epithelial cells when stimulated with PMA/Ionomycin (FIG. 11e). Comparable to group 3 ILC, the proportion of T-bet+ILC and the number of IFN-γ⁺ cells was greater in the absence of mesenchyme than in the absence of epithelium (FIG. 10e).

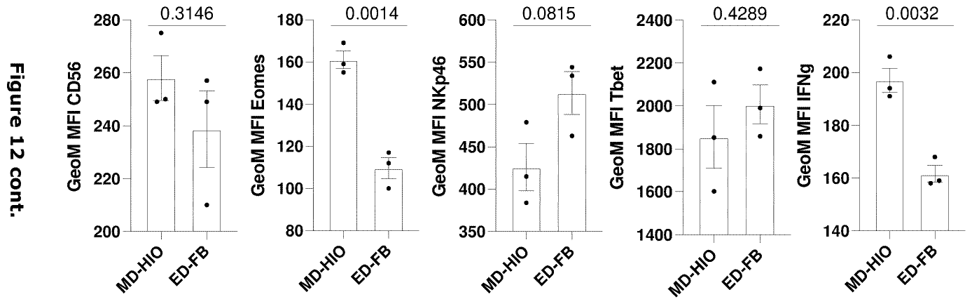

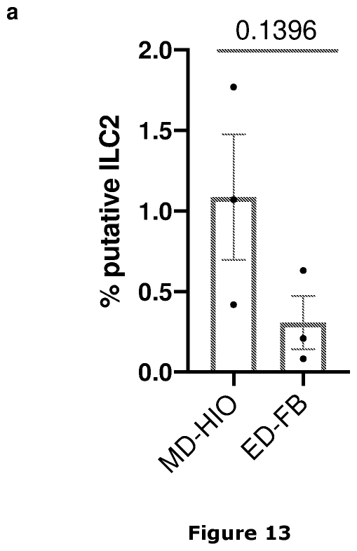

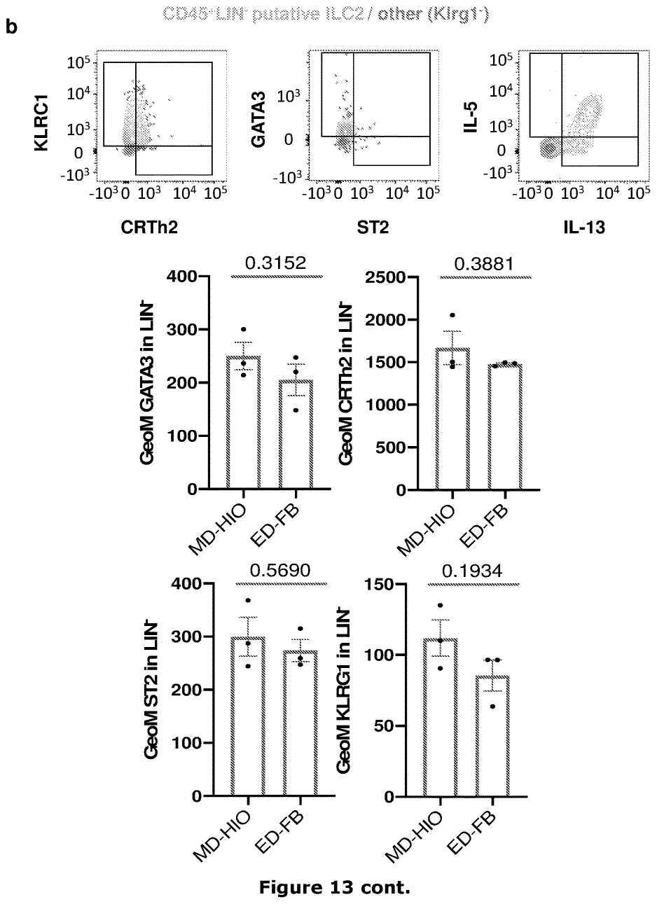

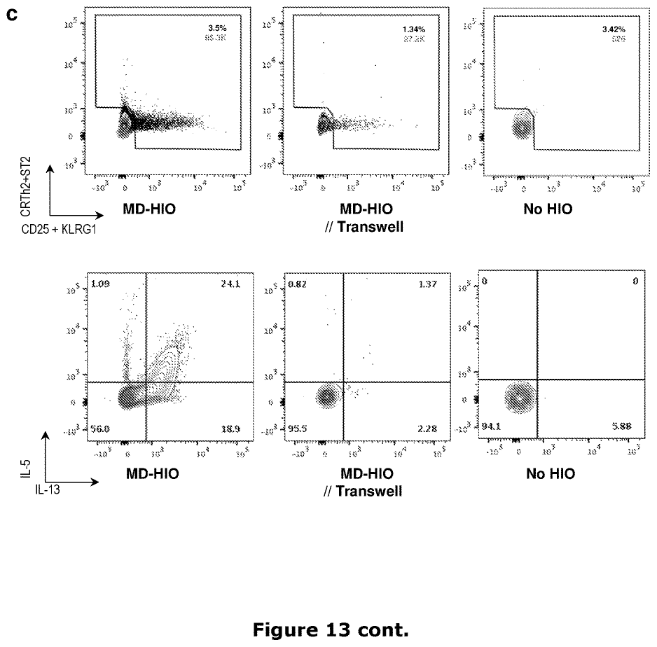

Unlike group 1 and 3 ILC, the overall count (FIG. 11f) and relative frequency (FIG. 13a) of putative group 2 ILC (FIG. 13b) was not significantly affected by the depletion of mesenchyme. Nevertheless, expression of IL-5 and IL-13 were both significantly and greatly increased in mesenchyme-depleted HIO fractions upon unbiased stimulation with PMA/Ionomycin (FIG. 11g). The lack of difference in ILC2 numbers induced by epithelial depletion stands in contrast to findings in murine ILC2P transwell cultures, where the ILC2 number was significantly decreased upon physical separation from epithelial cells (FIG. 9d-f). To assess whether contact-dependent mechanisms were conserved in maturing human ILCP-derived ILC2, ILCP were also separated from mesenchyme depleted HIO (FIG. 13c). Though the yield of ILC2 was still greater in transwell-separated co-cultures than in ILCP cultured without organoids, both the overall cell count and relative ILC2 frequency appeared to decrease in transwell culture, and these ILC2 failed to express IL-5 or IL-13 (FIG. 13c). Finally, we aimed to assess whether the lack of cytokine expression in epithelial-depleted co-cultures was because ILC2 failed to mature without epithelial cells, or if this was the result of active ILC2 cytokine repression by mesenchyme either through secretion of repressive factors or via competitive depletion of necessary ligands. To test this, ILCP were first successfully matured with MD-HIO, then resulting EpCAM⁻ CD45⁺ Lineage-ILC were FACS-purified and reseeded either with MD-HIO or swapped to ED-FB (FIG. 13d). ILC reseeded without epithelial cells appeared to decrease their rate of proliferation, and while there was no difference in the expression of IL-13 (FIG. 13e), the greater presence of mesenchyme resulted in a significant decrease in IL-5 expression by ILC2 (FIG. 13e). This suggests that stable development of IL-13⁺ ILC2 may be promoted by intestinal epithelial contact, but ILC2 proliferation and expression of IL-5 may either be actively repressed by intestinal mesenchyme or require constant epithelial exposure.

### Example 6: Human Mucosal Epithelial Identity Contributes to Tissue-Specific ILC Maturation

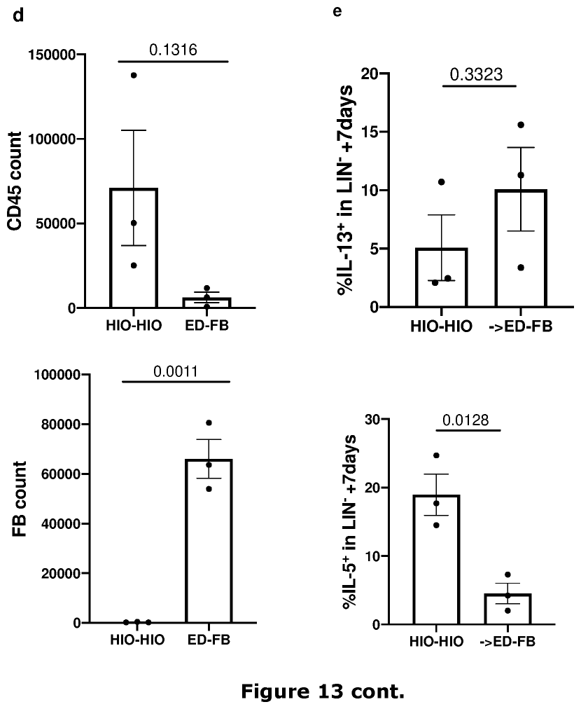

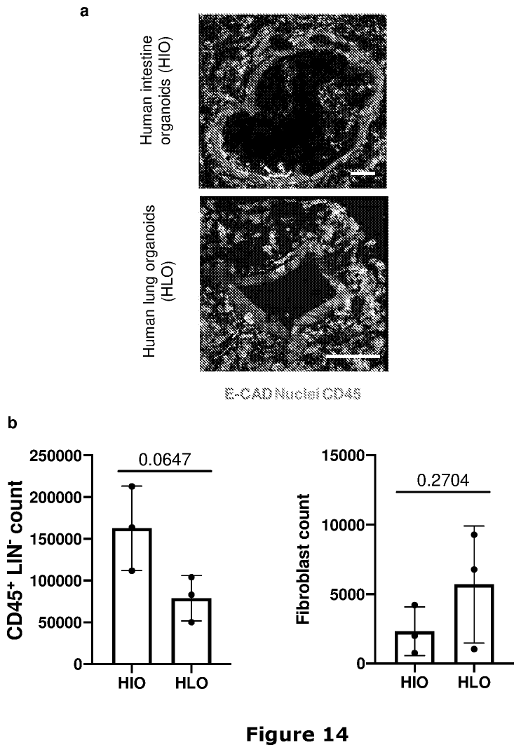

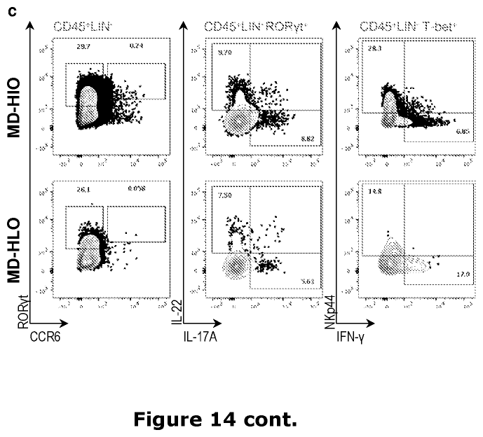

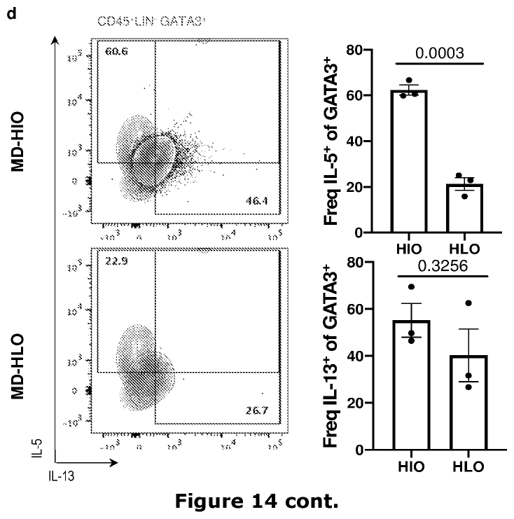

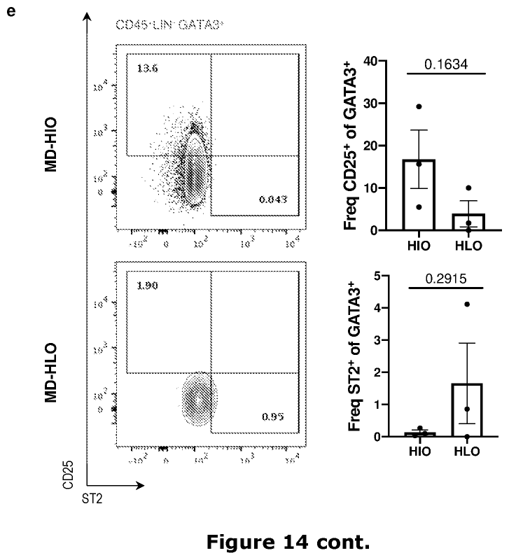

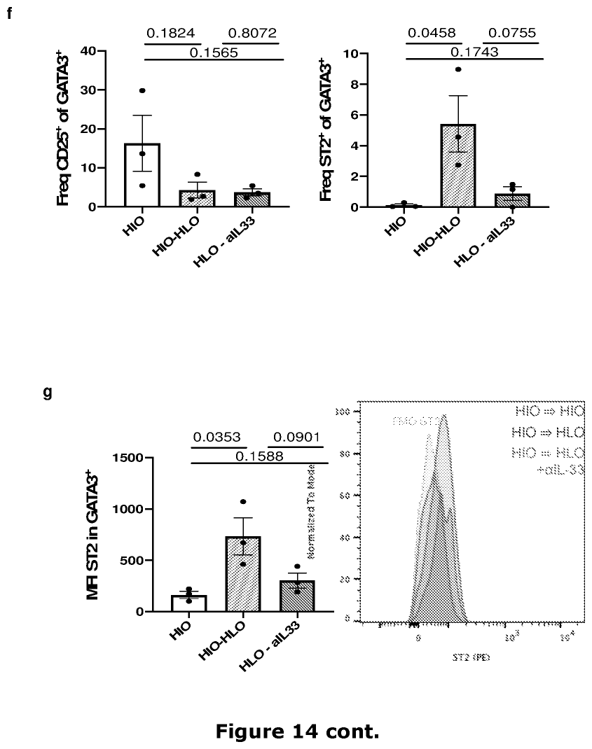

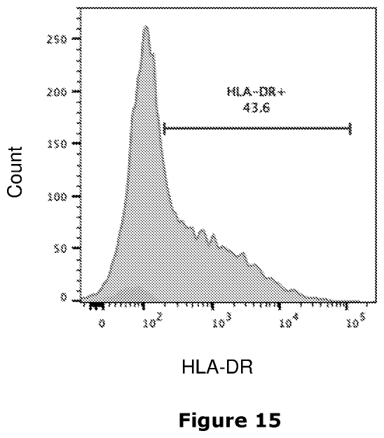

To assess if any tissue-characteristic ILC phenotypes could be captured in these human organoid systems (FIG. 14a), ILCP were cultured either with hiPSC-derived small intestine or hiPSC-derived lung organoids. ILCP expanded in both co-cultures (FIG. 14b), in which the absolute number of fibroblasts post mesenchyme-depletion remaining sufficiently consistent between conditions. HLO demonstrated comparable capacity to yield mature group 1 and group 3 cells that expressed IL-22, IL-17A, and IFN-γ (FIG. 14c). Both HIO and HLO yielded GATA3⁺ ILC that expressed IL-5 and IL-13 (FIG. 14d). Much like ED-FB, HLO did not support maturation of IL-5⁺ as significantly as HLO. Similarly, a recently described c-KIT⁺, CRTh2^{low}, IL-17A⁺ ILC2 population was additionally present in HIO, but not in HLO cultures, suggesting that much like the murine system, the human intestinal microenvironment may favour group 3 maturation, even in inducing ex-ILC2 to ILC3 plasticity in mature MD-HIO-derived ILC2. However, unlike in the murine system, co-culture with HIO or HLO alone was not sufficient to induce statistically significant differences in the frequency of CD25 and ST2 ILC within this GATA3+ putative ILC2 population (FIG. 14e). Finally, we assessed the capacity of the human organoids to recapitulate the gut-lung translocation approach introduced for the murine co-cultures (FIG. 6e). ILCP were matured with MD-HIO for two weeks, then FACS-purified and reseeded with MD-HIO, MD-HLO, or MD-HLO with human IL-33 neutralisation, as performed in the murine system. HiPSC-derived lung organoids did not significantly impact the frequency of CD25⁺ (FIG. 14f). However, the anticipated increase in pulmonary ILC2 ST2 expression that was not captured by HIO and HLO co-culture alone was significantly observed upon transfer of MD-HIO-derived ILC2 to lung culture (FIG. 14g). This effect was dampened by addition of neutralising IL-33.

### Example 7: Antigen Processing of the ILCs of the Invention

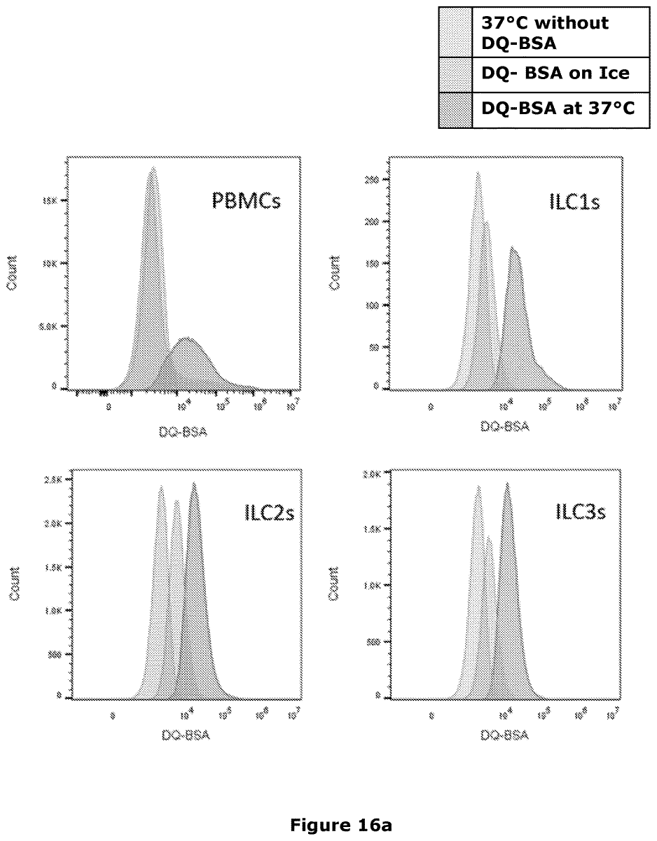

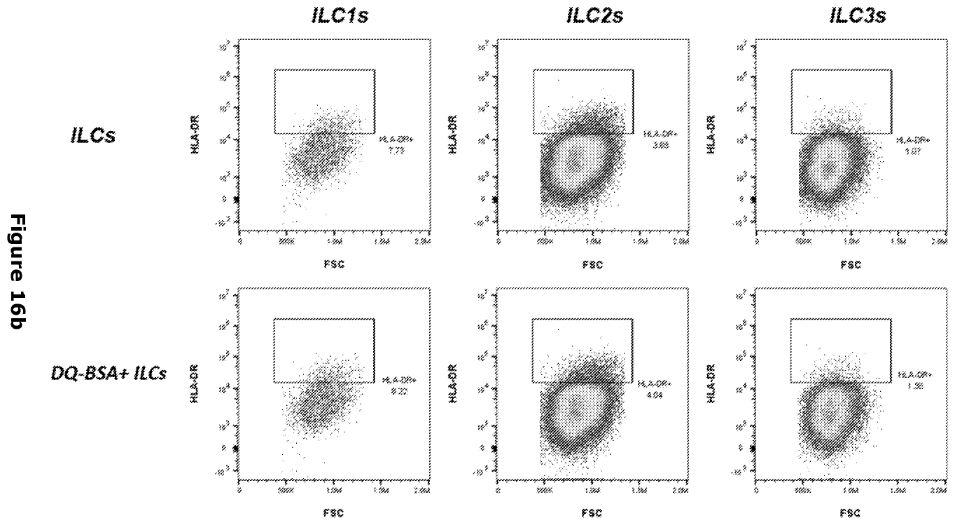

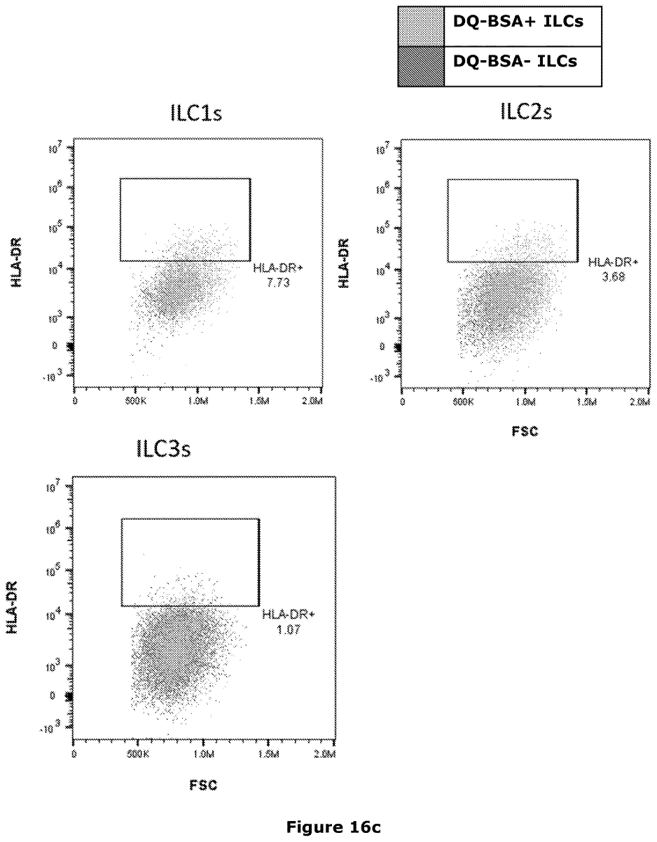

The ability of the ILCs of the invention to process antigens via MHC-II was tested by hydrolysis of DQ-BSA (which leads to fluorescent signal). The positive results are shown in FIG. 16.

### Example 8: Single Cell RNAseq (scRNAseq) on the Organoid-Generated ILCs

From the co-cultures, CD45+ Lin− cells were isolated via fluorescence activated cell sorting (FACS). We identified 5 ‘super-clusters’ of cells corresponding to a cytotoxic ILC1 population, two distinct ILC2 clusters (ILC2a and ILC2b), an ILC3 cluster, and a small cluster of cells which exhibited significantly upregulated expression of genes characteristic of regulatory T cells (Tregs) and a regulatory ILC (ILCreg) population previously described in mice (Wang et al., 2017) (FIG. 17A).

The helper-like ILC cell types (ILC1-3) were identified via determining expression patterns of key genes previously described to distinguish the different ILC family members in single cell RNA sequencing studies using human samples (Björklund et al., 2016; Cella et al., 2019; Liu et al., 2021; Mazzurana et al., 2021). The significant enrichment of these genes in each cluster was determined via unbiased differential gene expression analysis comparing expression of the genes within each cluster to that of all other clusters (FIG. 17B). Accordingly, the ILC1 population was identified via its enriched expression of key cytokines and chemokines including IFNG and CCL3 alongside their relatively high expression of genes such KLRC1, PRF1, and GNLY, previously shown to characterise cytotoxic populations of ILC1. The increased expression of the transcription factor GATA-3 (encoded via GATA3) and the cytokine signalling genes IL33 and IL17RB were used to identify the two ILC2 populations, alongside their upregulation of additional ILC2-associated genes such as HPGDS and KLRG1. Finally, ILC3s showed significantly greater enrichment of genes encoding their characteristic transcription factors (e.g., RORC), cell surface markers (e.g., KIT), and cytokine signalling pathways (e.g., IL23RA). These genes were found coupled to expression of markers previously shown to be highly expressed in both ILC3s and ILC1s, including NCR1 and PECAM1, further confirming the identity of both clusters.

The annotation of the ILCreg cluster, however, was primarily performed using markers identified in mouse research on ILCregs as this population has yet to be extensively characterised in human patients, although an IL-10 expressing ILC population was identified in the human intestine (Wang et al., 2017). This included genes encoding their key cytokine, IL-10 (IL10), the cell surface marker CD25 (IL2RA) and the transcription factors ID3 and SOX4. Interestingly, the ILCreg cluster additionally expressed genes associated with human T-regulatory (Tregs) cells including FOXP3, RUNX1, IL1R1 and CTLA4. Importantly, the T-cell markers CD4 and CD3 were included within the lineage gating panel when sorting the ILCs from the co-cultures, and the lack of their expression was further confirmed at the RNA level. Accordingly, these cells were deemed to be the human ILC equivalent of Tregs.

Using cell-specific enrichment scores for a human ILCP gene signature (Liu et al., 2021) we identified a small population of these precursor cells within the larger ILC3 super-cluster (FIG. 17C). Moreover, the cytotoxic ILC1 super-cluster showed distinct expression patterns of the transcription factors TBX21 and EOMES, suggesting this contained both ILC1 and NK-like cells (FIG. 17D). Overall, we found cells corresponding to the major ILC cell types expected to be observed in our co-culture model, alongside a putative population of human ILCregs.

To determine whether the ILCs exhibited an intestinal tissue specific phenotype, we generated cell-specific enrichment scores for three gene modules identified by Mazzurana et al., (2021) associated with blood/tonsillar circulatory ILCs (mod11), colonic ILCs (mod3) or lung ILCs (mod34) (Mazzurana et al., 2021). Only ILC3s were examined here as Mazzurana et al., did not identify any distinct clusters of ILC1s or ILC2s in the colon. The ILC3 super-cluster was found to exhibit the greatest enrichment for the circulatory gene module (FIG. 17E), followed by the colonic gene module, with minimal cells showing enriched expression of lung ILC associated genes. Considering we observed the putative ILCP population within our ILC3s, we subsequently overlaid the enrichment scores onto the UMAP to determine any heterogeneity in the pattern of expression of these gene modules (FIG. 17F). This revealed that the ILC super-cluster could be clearly differentiated into those cells which exhibit a more circulatory phenotype compared to those which express a gene signature more closely resembling that of colonic ILC3s. As expected, there were minimal numbers of cells enriched for the lung module. Taken together, these data suggest that a substantial proportion of the ILC3s in this co-culture model differentiate away from a circulatory phenotype to acquire a colonic gene expression signature.

### Experiment Outline:

Circulatory innate lymphoid cell precursors (ILCPs; CD45+ Lin− CD56− CD127+ CRTh2− cKIT+ NKp46− KLRG1−) were isolated from the blood of age-matched male and female donors (n=1), placed into co-culture with iPSC-derived human intestinal organoids and allowed to expand for 14 days. After this 14-day period, four populations of ILCs were isolated from the co-cultures using FACS: ILC2 (CD45+ Lin− CRTh2+), ILC3 (CD45+ Lin− CRTh2+ cKIT+), ILC1/NK cells (CD45+ Lin− CRTh2− cKIT− CD56+/− CD161+) and ‘other’ ILC (CD45+ Lin− CRTh2− cKIT− CD56+/− CD161−). Each population was subsequently pooled in a 1:1 ratio of male to female cells to give a final total proportion of each ILC population at 25, 15, 35 and 15% respectively. These proportions were decided upon based on prior knowledge of the heterogeneity of each ILC subset, with those ILCs displaying greater heterogeneity contributing a larger percentage of the final cell counts. This pool of cells was then sequenced as single sample via 10× sequencing.

scRNA-Seq Data Processing and Analysis:

Barcoding, cDNA synthesis, and library preparation were performed using Chromium™ Next GEM Single Cell 3′, Library & Gel Bead Kit v3.1 according to the manufacturer's instructions. Libraries were sequenced by Novogene using NovaSeq with a target of 20,000 reads per cell. Pre-processing of single cell RNA-sequencing fastq files was performed using Cell Ranger v7.0.1 (10× Genomics), with reads aligned to the GRCh38 reference genome. Further data pre-processing was performed in R (v4.2.2) using the package Seurat (v4.3.0). Cells with less than 1,000 genes and greater than 10% of transcripts derived from mitochondrial genes were removed as they were considered lysed/apoptotic. Moreover, cells with more than 7,500 genes or 40,000 UMIs were excluded as these were presumed to contain doublets. The filtered dataset was subsequently normalised using the Seurat SCTransform function (v2) in which the effects of the cell cycle were regressed out. The top 30 principal components were then used as input for Louvain clustering via the FindClusters( ) function in Seurat at resolution 0.2. UMAP was used for cluster visualisation. Differentially expressed genes were determined using the Wilcox test in the FindAllMarkers( ) function in Seurat, and genes with log fold change >=2.5 and a false discovery rate P<0.05 were considered significantly differentially expressed. To visualise lowly expressed genes (TBX21 and EOMES) the plot_density function was used from the R package Nebulosa (v1.8.0). To determine the enrichment of different ILC gene signatures, the R package AUCell (v1.6.1) was used and the resultant scores were plotted for visualisation.

The results are shown in FIG. 17 and the Table 5 below.

TABLE 5
Results of scRNAseq) on the organoid-generated ILCs

| ILC1/NK cell | ILC2 | ILC3 | ILCreg |
|---|---|---|---|
| genes | genes | genes | genes |
| AOAH | ANXA1 | AHR | CCR5 |
| CCL3 | BCL11B | BCL2A1 | CTLA4 |
| CCL4 | CCR2 | CCL20 | FGGY |
| CCL5 | GATA3 | CD81 | FOXP3 |
| CD244 | HPGD | CSF2 | GATA3 |
| CD247 | HPGDS | CXCL8 | GZMB |
| CST7 | IL10RA | ID2 | IL10 |
| EOMES | IL13 | IKZF2 | IL1R1 |
| FCGR3A | IL17RB | IL1R1 | IL2RA |
| FGR | IL32 | IL23R | IL2RB |
| GNLY | IL5 | IL4I1 | KAT2B |
| GZMB | IL9R | IRF4 | LGLS3 |
| GZMK | KLRG1 | KIT | PIM1 |
| IFNG | LGALS1 | LIF | PRDM1 |
| IKZF3 | MAF | LTA4H | RUNX1 |
| IL12RB2 | MBOAT2 | NCR1 | SOX4 |
| ITGAX | PPARG | PECAM1 | TNFRSF18 |
| ITGB2 | PTGDR2 | RBPJ | TRAF1 |
| KLRC1 | PTGER2 | RORC | |
| KLRD1 | TNFSF10 | TNFRSF25 | |
| NCAM1 | | TNFSF4 | |
| NCR1 | | TOX2 | |
| NKG7 | | | |
| PRF1 | | | |
| SAMD3 | | | |
| TBX21 | | | |
| TIGIT | | | |
| ZNF683 | | | |

### Example 9: NK Cells Generated by Co-Culture of ILC Precursors (ILCP) with Intestinal Organoids are Cytotoxic

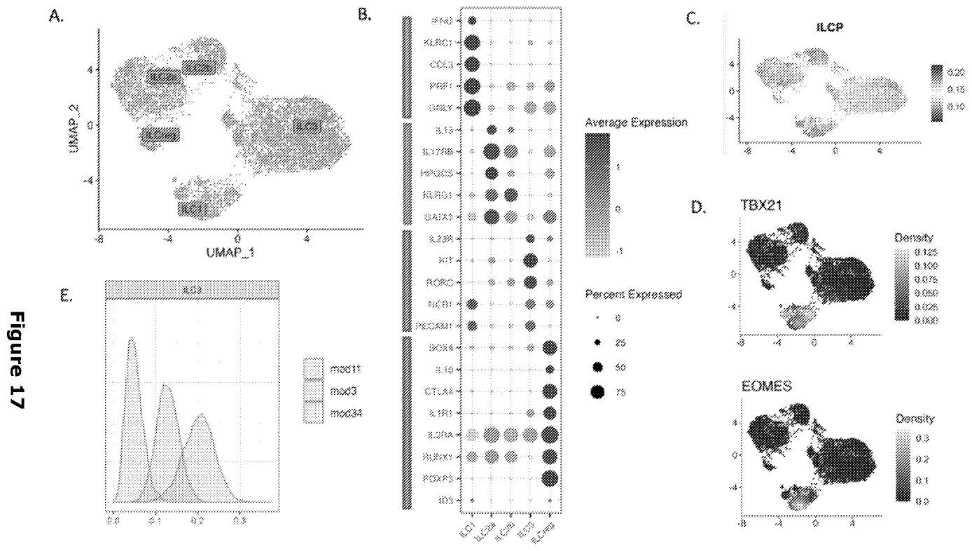

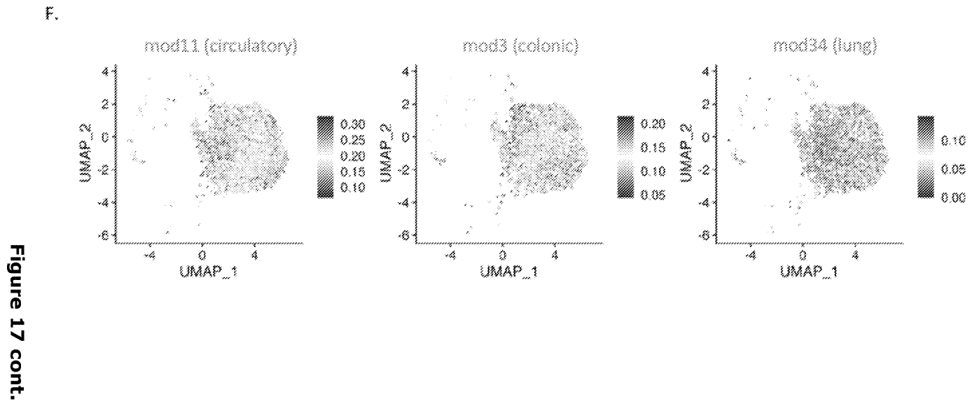

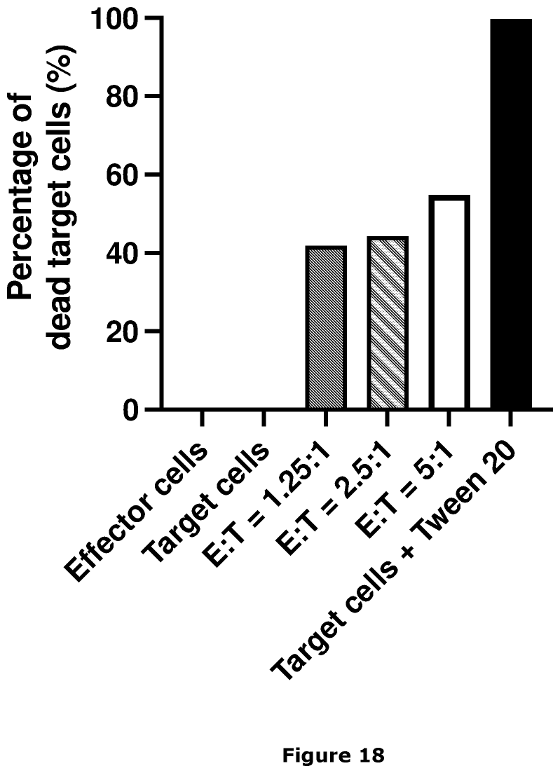

The cytotoxicity of NK cells generated by co-culture of ILC precursors (ILCP) with intestinal organoids was tested using standard methods. The positive results are shown in FIG. 18.

### Example 10: Generation of ILCs in Human Small Intestine Biopsy Derived Organoids

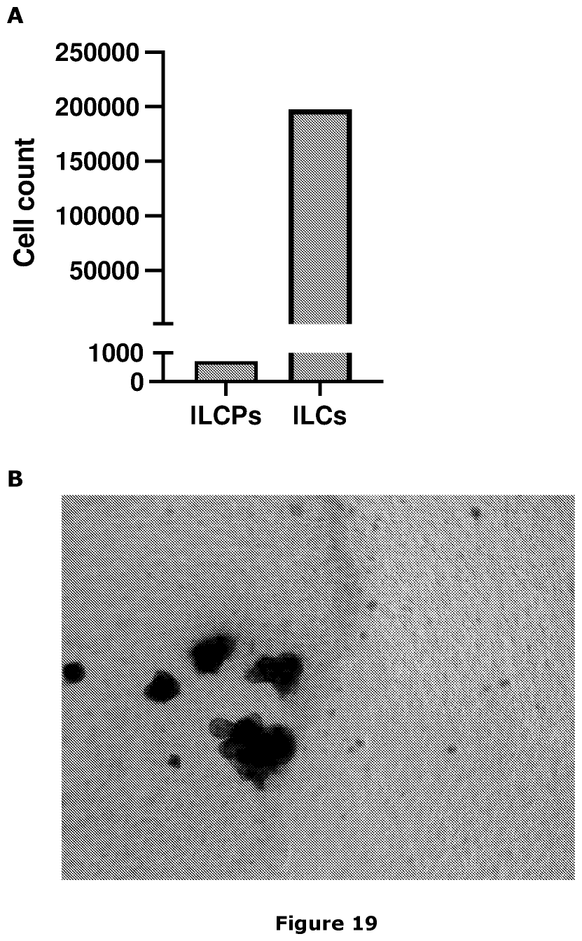

Differentiated ILCs (including ILC1, ILC2, ILC3 and NK cells) were generated from ILC precursors (ILCP) after 15 days in culture with human small intestine biopsy derived organoids. The results are shown in FIG. 19.

### REFERENCES

Bando, J. K., Liang, H. E., and Locksley, R. M. (2015). Identification and distribution of developing innate lymphoid cells in the fetal mouse intestine. Nat. Immunol. 16, 153-160.
Bando J K, Gilfillan S, Di Luccia B, Fachi J L, Secca C, Cella M, Colonna M. ILC2s are the predominant source of intestinal ILC-derived IL-10. J Exp Med. 2020 Feb. 3; 217 (2): e20191520. doi: 10.1084/jem.20191520. PMID: 31699824; PMCID: PMC7041711
Björklund, A. K., Forkel, M., Picelli, S., Konya, V., Theorell, J., Friberg, D., Sandberg, R., & Mjösberg, J. (2016). The heterogeneity of human CD127+innate lymphoid cells revealed by single-cell RNA sequencing. Nature Immunology 2016 17:4, 17 (4), 451-460. https://doi.org/10.1038/ni.3368.
Cella, M., Gamini, R., Sécca, C., Collins, P. L., Zhao, S., Peng, V., Robinette, M. L., Schettini, J., Zaitsev, K., Gordon, W., Bando, J. K., Yomogida, K., Cortez, V., Fronick, C., Fulton, R., Lin, L. L., Gilfillan, S., Flavell, R. A., Shan, L., . . . . Colonna, M. (2019). Subsets of ILC3-ILC1-like cells generate a diversity spectrum of innate lymphoid cells in human mucosal tissues. Nature Immunology 2019 20:8, 20 (8), 980-991. https://doi.org/10.1038/s41590-019-0425-y.
Dutton, E. E., Camelo, A., Sleeman, M., Herbst, R., Carlesso, G., Belz, G. T., and Withers, D. R. (2018). Characterisation of innate lymphoid cell populations at different sites in mice with defective T cell immunity [version 3; referees: 2 approved]. Wellcome Open Res. 2, 117.
Elmentaite, R., Kumasaka, N., Roberts, K., Fleming, A., Dann, E., King, H. W., Kleshchevnikov, V., Dabrowska, M., Pritchard, S., Bolt, L., et al. (2021). Cells of the human intestinal tract mapped across space and time. Nature 597, 250-255.
Gronke, K., Kofoed-Nielsen, M., and Diefenbach, A. (2017). Isolation and Flow Cytometry Analysis of Innate Lymphoid Cells from the Intestinal Lamina Propria. In Methods in Molecular Biology (Clifton, N.J.), pp. 255-265.
Heo, I., Dutta, D., Schaefer, D. A., Iakobachvili, N., Artegiani, B., Sachs, N., Boonekamp, K. E., Bowden, G., Hendrickx, A. P. A., Willems, R. J. L., et al. (2018). Modelling Cryptosporidium infection in human small intestinal and lung organoids. Nat. Microbiol. 3, 814-823.
Hernández, D. C., Juelke, K., Müller, N. C., Durek, P., Ugursu, B., Mashreghi, M. F., Rückert, T., and Romagnani, C. (2021). An in vitro platform supports generation of human innate lymphoid cells from CD34+ hematopoietic progenitors that recapitulate ex vivo identity. Immunity 54, 2417-2432.e5.
Huang, Y., Guo, L., Qiu, J., Chen, X., Hu-Li, J., Siebenlist, U., Williamson, P. R., Urban, J. F., and Paul, W. E. (2015). IL-25-responsive, lineage-negative KLRG1 hi cells are multipotential “inflammatory” type 2 innate lymphoid cells. Nat. Immunol. 16, 161-169.
Huang, Y., Mao, K., Chen, X., Sun, M. A., Kawabe, T., Li, W., Usher, N., Zhu, J., Urban, J. F., Paul, W. E., et al. (2018). S1P-dependent interorgan trafficking of group 2 innate lymphoid cells supports host defense. Science (80-.). 359, 114-119.
Jowett, G. M., Norman, M. D. A., Yu, T. T. L. et al. ILC1 drive intestinal epithelial and matrix remodelling. Nat. Mater. 20, 250-259 (2021). https://doi.org/10.1038/s41563-020-0783-8
Kilpinen, H., Goncalves, A., Leha, A., Afzal, V., Alasoo, K., Ashford, S., Bala, S., Bensaddek, D., Casale, F. P., Culley, O. J., et al. (2017). Common genetic variation drives molecular heterogeneity in human iPSCs. Nature 546, 370-375.
Konishi, S., Gotoh, S., Tateishi, K., Yamamoto, Y., Korogi, Y., Nagasaki, T., Matsumoto, H., Muro, S., Hirai, T., Ito, I., et al. (2016). Directed Induction of Functional Multi-ciliated Cells in Proximal Airway Epithelial Spheroids from Human Pluripotent Stem Cells. Stem Cell Reports 6, 18-25.
Krämer, B., Goeser, F., Lutz, P., Glassner, A., Boesecke, C., Schwarze-Zander, C., Kaczmarek, D., Nischalke, H. D., Branchi, V., Manekeller, S., et al. (2017). Compartment-specific distribution of human intestinal innate lymphoid cells is altered in HIV patients under effective therapy. PLOS Pathog. 13.
Leha, A., Moens, N., Meleckyte, R., Culley, O. J., Gervasio, M. K., Kerz, M., Reimer, A., Cain, S. A., Streeter, I., Folarin, A., et al. (2016). A high-content platform to characterise human induced pluripotent stem cell lines. Methods 96, 85-96.
Lim, A. I., Li, Y., Lopez-Lastra, S., Stadhouders, R., Paul, F., Casrouge, A., Serafini, N., Puel, A., Bustamante, J., Surace, L., et al. (2017a). Systemic Human ILC Precursors Provide a Substrate for Tissue ILC Differentiation. Cell 168, 1086-1100.e10.
Lim, A. I., Li, Y., Lopez-Lastra, S., Stadhouders, R., Paul, F., Casrouge, A., Serafini, N., Puel, A., Bustamante, J., Surace, L., et al. (2017b). Systemic Human ILC Precursors Provide a Substrate for Tissue ILC Differentiation. Cell 168, 1086-1100.e10.
Liu, C., Gong, Y., Zhang, H., Yang, H., Zeng, Y., Bian, Z., Xin, Q., Bai, Z., Zhang, M., He, J., Yan, J., Zhou, J., Li, Z., Ni, Y., Wen, A., Lan, Y., Hu, H., & Liu, B. (2021). Delineating spatiotemporal and hierarchical development of human fetal innate lymphoid cells. Cell Research, 31 (10). https://doi.org/10.1038/s41422-021-00529-2.
Mazzurana, L., Czarnewski, P., Jonsson, V., Wigge, L., Ringner, M., Williams, T. C., Ravindran, A., Björklund, Å. K., Säfholm, J., Nilsson, G., Dahlén, S. E., Orre, A. C., Al-Ameri, M., Höög, C., Hedin, C., Szczegielniak, S., Almer, S., & Mjösberg, J. (2021). Tissue-specific transcriptional imprinting and heterogeneity in human innate lymphoid cells revealed by full-length single-cell RNA-sequencing. Cell Research 2021 31:5, 31 (5), 554-568. https://doi.org/10.1038/s41422-020-00445-x.
McCracken, K. W., Howell, J. C., Wells, J. M., and Spence, J. R. (2011). Generating human intestinal tissue from pluripotent stem cells in vitro. Nat. Protoc. 6, 1920-1928.
McQualter, J. L., Yuen, K., Williams, B., and Bertoncello, I. (2010). Evidence of an epithelial stem/progenitor cell hierarchy in the adult mouse lung. Proc. Natl. Acad. Sci. U.S.A 107, 1414-1419.
Nakano, T. (1996). In vitro development of hematopoietic system from mouse embryonic stem cells: A new approach for embryonic hematopoiesis. Int. J. Hematol. 65, 1-8.
Ricardo-Gonzalez, R. R., Van Dyken, S. J., Schneider, C., Lee, J., Nussbaum, J. C., Liang, H. E., Vaka, D., Eckalbar, W. L., Molofsky, A. B., Erle, D. J., et al. (2018a). Tissue signals imprint ILC2 identity with anticipatory function. Nat. Immunol. 19, 1093-1099.
Saluzzo, S., Gorki, A. D., Rana, B. M. J., Martins, R., Scanlon, S., Starkl, P., Lakovits, K., Hladik, A., Korosec, A., Sharif, O., et al. (2017). First-Breath-Induced Type 2 Pathways Shape the Lung Immune Environment. Cell Rep. 18, 1893-1905.
Sato, T., Vries, R. G., Snippert, H. J., van de Wetering, M., Barker, N., Stange, D. E., van Es, J. H., Abo, A., Kujala, P., Peters, P. J., et al. (2009). Single Lgr5 stem cells build crypt-villus structures in vitro without a mesenchymal niche. Nature 459, 262-265.
Sato, T., van Es, J. H., Snippert, H. J., Stange, D. E., Vries, R. G., van den Born, M., Barker, N., Shroyer, N. F., van de Wetering, M., and Clevers, H. (2011). Paneth cells constitute the niche for Lgr5 stem cells in intestinal crypts. Nature 469, 415-418.
Schroeder, J.-H., Meissl, K., Hromadová, D., Lo, J. W., Neves, J. F., Howard, J. K., Helmby, H., Powell, N., Strobl, B., and Lord, G. M. (2021). T-Bet Controls Cellularity of Intestinal Group 3 Innate Lymphoid Cells. Front. Immunol. 11, 3701.
Stallmach, A., Hahn, U., Mekker, H. J., Hahn, E. G., and Riecken, E. O. (1989). Differentiation of rat intestinal epithelial cells is induced by organotypic mesenchymal cells in vitro. Gut 30, 959-970.
Wang, S., Xia, P., Chen, Y., Qu, Y., Xiong, Z., Ye, B., Du, Y., Tian, Y., Yin, Z., Xu, Z., & Fan, Z. (2017). Regulatory Innate Lymphoid Cells Control Innate. Intestinal Inflammation. Cell, 171 (1). https://doi.org/10.1016/j.cell.2017.07.027
Xu, W., Domingues, R. G., Fonseca-Pereira, D., Ferreira, M., Ribeiro, H., Lopez-Lastra, S., Motomura, Y., Moreira-Santos, L., Bihl, F., Braud, V., et al. (2015). NFIL3 Orchestrates the emergence of common helper innate lymphoid cell precursors. Cell Rep. 10, 2043-2054.
Yusa, K., Rashid, S. T., Strick-Marchand, H., Varela, I., Liu, P. Q., Paschon, D. E., Miranda, E., Ordóñez, A., Hannan, N. R. F., Rouhani, F. J., et al. (2011). Targeted gene correction of α1-antitrypsin deficiency in induced pluripotent stem cells. Nature 478, 391-394.

1. A method for expanding immune cells, the method comprising:
(a) co-culturing the immune cells and at least one epithelial organoid, wherein the epithelial organoid comprises more epithelial cells than mesenchymal cells.

2. The method of claim 1, wherein the epithelial organoid comprises less than 45% mesenchymal cells.

3. The method of claim 1 or claim 2, wherein the epithelial organoid comprises less than 30% mesenchymal cells.

4. The method of any one of the preceding claims, wherein the epithelial organoid does not comprise a detectable level of mesenchymal cells.

5. The method of any one of the preceding claims, wherein the method comprises before step (a) a step of depleting mesenchymal cells from the epithelial organoid.

6. The method of any one of claims 1 to 4, wherein the epithelial organoid has undergone depletion of mesenchymal cells.

7. The method of any one of the preceding claims, wherein the epithelial organoid is a human or murine epithelial organoid.

8. The method of any one of the preceding claims, wherein the epithelial organoid is derived from induced pluripotent stem cells (iPSCs).

9. The method of any one of claims 1 to 7, wherein the epithelial organoid is a primary epithelial organoid.

10. The method of any one of the preceding claims, wherein the epithelial organoid is a skin, intestinal, lung, thymic, thyroid, reproductive, bladder, kidney, pancreas, oral mucosal or liver organoid.

11. The method of any one of the preceding claims, wherein the epithelial organoid is an intestinal epithelial organoid or lung epithelial organoid.

12. The method of any one of the preceding claims, wherein the immune cells are haematopoietic stem cells and/or lymphoid precursor cells.

13. The method of any one of claims 1 to 11, wherein the immune cells are lymphoid and/or myeloid cells.

14. The method of claim 13, wherein the immune cells are T-cells, B-cells and/or innate lymphoid cells (ILC).

15. The method of claim 14, wherein the immune cells are ILCs.

16. The method of claim 14 or claim 15, wherein the ILCs comprise one or more of Group 1, Group 2 and Group 3 ILCs or one or more of Group 1 ILCs, Group 2 ILC, Group 3 ILCs and regulatory ILCs (ILCregs).

17. The method of any one of the preceding claims, wherein the immune cells comprise a plurality of different immune cell subsets, optionally ILCs from a plurality of different ILC groups.

18. The method of claim 17, wherein one or more of the different immune cell subsets in the plurality are enriched over time.

19. The method of any one of the preceding claims, wherein the method is for the production and expansion of immune cells and wherein the method comprises before step (a):
co-culturing immune cell precursors and the at least one epithelial organoid to expand the immune cell precursors and differentiate the immune cell precursors into the immune cells.

20. The method of claim 19, wherein the immune cell precursors are lymphoid precursors.

21. The method of claim 19 or claim 20, wherein the immune cell precursors comprise isolated ILC precursors and the immune cells are ILCs.

22. The method of any one of the preceding claims, wherein the immune cells are primary immune cells.

23. The method of any one of the preceding claims, wherein the immune cells are human immune cells.

24. The method of any one of the preceding claims, wherein the immune cells and the at least one epithelial organoid are co-cultured for at least about 72 hours.

25. The method of any one of the preceding claims, wherein the immune cells expand at a rate of at least 2 fold every 24 hours.

26. Immune cells obtainable by the method of any one of claims 1 to 25.

27. An in vitro population of innate lymphoid cells (ILCs), wherein the population comprises at least 5×10³ ILCs.

28. The in vitro population of ILCs of claim 27, wherein the ILCs comprise one or more of Group 1, Group 2 and Group 3 ILCs.

29. The in vitro population of ILCs of claim 28, wherein the ILCs comprise ILC1 cells and/or NK cells.

30. The in vitro population of ILCs of any one of claims 27 to 29, wherein at least 10% of the population of ILCs express a detectable level of NKp44.

31. The in vitro population of ILCs of any one of claims 27 to 30, wherein the population comprises ILCs from a plurality of different ILC groups.

32. The in vitro population of ILCs of any one of claims 27 to 31, wherein at least 10% of the in vitro population comprise an exogenous polynucleotide.

33. A pharmaceutical composition comprising the immune cells of claim 26 or the in vitro population of any one of claims 27 to 32 and a pharmaceutically or physiologically acceptable diluent and/or carrier.

34. A method of treating or preventing a disease in a subject, wherein the method comprises administering to the subject the immune cells of claim 26, the in vitro population of any one of claims 27 to 32 or the pharmaceutical composition of claim 33.

35. The immune cells of claim 26, the in vitro population of any one of claims 27 to 32 or the pharmaceutical composition of claim 33 for use in the treatment or prevention of a disease.
